SEQUENCE LISTING (71 sequences)

SEQ ID NO: 1 - protein, 262 aa, Artificial Sequence
  Description of Artificial Sequence Synthetic polypeptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 2 - DNA, 786 bp, Artificial Sequence
  Description of Artificial Sequence Synthetic polynucleotide
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctcta

SEQ ID NO: 3 - protein, 415 aa, Artificial Sequence
  Description of Artificial Sequence Synthetic polypeptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 1245 bp, Artificial Sequence
  Description of Artificial Sequence Synthetic polynucleotide
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctctagatctcgagcccaaatcttgtgacaaaactcacacatgcccaccgtgcccggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5 - protein, 462 aa, Artificial Sequence
  Description of Artificial Sequence Synthetic polypeptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 1386 bp, Artificial Sequence
  Description of Artificial Sequence Synthetic polynucleotide
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctctagatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 7 - protein, 267 aa, Artificial Sequence
  Description of Artificial Sequence Synthetic polypeptide
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 8 - DNA, 801 bp, Artificial Sequence
  Description of Artificial Sequence Synthetic polynucleotide
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaag

SEQ ID NO: 9 - protein, 467 aa, Artificial Sequence
  Description of Artificial Sequence Synthetic polypeptide
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1401 bp, Artificial Sequence
  Description of Artificial Sequence Synthetic polynucleotide
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 11 - protein, 323 aa, Artificial Sequence
  Description of Artificial Sequence Synthetic polypeptide
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKDLEPKSCDKTHTCPPCPDPKFWVLVVVGGVLACYSLLVTVAFIIFWVRSKRSRLLH

SEQ ID NO: 12 - DNA, 969 bp, Artificial Sequence
  Description of Artificial Sequence Synthetic polynucleotide
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcac

SEQ ID NO: 13 - protein, 810 aa, Artificial Sequence
  Description of Artificial Sequence Synthetic polypeptide
MDWIWRILFLVGAATGAHSQVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 14 - DNA, 2433 bp, Artificial Sequence
  Description of Artificial Sequence Synthetic polynucleotide
atggactggatctggcggattctgttcctcgtgggagccgccacaggcgctcactcacaggtgcagctgctggaatctggcggcggactggtgcagcctggcggcagcctgagactgagctgcgccgccagcggcttcaccttcagcagctacaccatgagctgggtccggcaggctcctggacaggccctggaatggatgggcaccatcagcagcggcggcacctacacctactaccccgacagcgtgaagggccggttcaccatcagccgggacaacgccaagaacagcctgtacctgcagatgaacagcctgagagccgaggacacagccgtgtactactgcgccagagaggccatcttcacctactggggcagaggcaccctggtcacaagcagcggaggcggaggaagtggagggggaggatcaggcggcggaggcagcgatatccagctgacccagagccctagcagcctgagcgccagcgtgggcgacagagtgaccatcacatgcaaggccagccaggacatcaacaactacctgagctggtatcagcagaagcccggccaggcccccagactgctgatctaccgggccaacagactggtggacggcgtgcccgatagattcagcggcagcggctacggcaccgacttcaccctgaccatcaacaacatcgagtccgaggacgccgcctactacttctgcctgaagtacgacgtgttcccctacaccttcggccagggcaccaaggtggagatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 15 - protein, 19 aa, Artificial Sequence
  Description of Artificial Sequence Synthetic peptide
MDWIWRILFLVGAATGAHS

SEQ ID NO: 16 - DNA, 57 bp, Artificial Sequence
  Description of Artificial Sequence Synthetic oligonucleotide
atggactggatctggcgcatcctgtttctcgtgggagccgccacaggcgcccattct

SEQ ID NO: 17 - protein, 243 aa, Artificial Sequence
  Description of Artificial Sequence Synthetic polypeptide
EVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 18 - DNA, 729 bp, Artificial Sequence
  Description of Artificial Sequence Synthetic polynucleotide
gaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctcta

SEQ ID NO: 19 - protein, 17 aa, Artificial Sequence
  Description of Artificial Sequence Synthetic peptide
DLEPKSCDKTHTCPPCP

SEQ ID NO: 20 - DNA, 51 bp, Artificial Sequence
  Description of Artificial Sequence Synthetic oligonucleotide
gatctcgagcccaaatcttgtgacaaaactcacacatgcccaccgtgcccg

SEQ ID NO: 21 - protein, 20 aa, Artificial Sequence
  Description of Artificial Sequence Synthetic peptide
DLEPKSCDKTHTCPPCPDPK

SEQ ID NO: 22 - DNA, 60 bp, Artificial Sequence
  Description of Artificial Sequence Synthetic oligonucleotide
gatctcgagcccaaatcttgtgacaaaactcacacatgcccaccgtgcccggatcccaaa

SEQ ID NO: 23 - protein, 21 aa, Artificial Sequence
  Description of Artificial Sequence Synthetic peptide
LCYLLDGILFIYGVILTALFL

SEQ ID NO: 24 - DNA, 63 bp, Artificial Sequence
  Description of Artificial Sequence Synthetic oligonucleotide
ctctgctacctgctggatggaatcctcttcatctatggtgtcattctcactgccttgttcctg

SEQ ID NO: 25 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 336 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - protein, 133 aa, Artificial Sequence
  Description of Artificial Sequence Synthetic polypeptide
LCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 399 bp, Artificial Sequence
  Description of Artificial Sequence Synthetic polynucleotide
ctctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - protein, 248 aa, Artificial Sequence
  Description of Artificial Sequence Synthetic polypeptide
QVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 30 - DNA, 744 bp, Artificial Sequence
  Description of Artificial Sequence Synthetic polynucleotide
caggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaag

SEQ ID NO: 31 - protein, 27 aa, Artificial Sequence
  Description of Artificial Sequence Synthetic peptide
FWVLVVVGGVLACYSLLVTVAFIIFWV

SEQ ID NO: 32 - DNA, 81 bp, Artificial Sequence
  Description of Artificial Sequence Synthetic oligonucleotide
ttttgggtgctggtggtggttggtggagtcctggcttgctatagcttgctagtaacagtggcctttattattttctgggtg

SEQ ID NO: 33 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 34 - DNA, 123 bp, Artificial Sequence
  Description of Artificial Sequence Synthetic polynucleotide
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 35 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 36 - DNA, 204 bp, Artificial Sequence
  Description of Artificial Sequence Synthetic polynucleotide
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 37 - DNA, 57 bp, Artificial Sequence
  Description of Artificial Sequence Synthetic oligonucleotide
atggactggatctggcggattctgttcctcgtgggagccgccacaggcgctcactca

SEQ ID NO: 38 - protein, 236 aa, Artificial Sequence
  Description of Artificial Sequence Synthetic polypeptide
QVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 39 - DNA, 708 bp, Artificial Sequence
  Description of Artificial Sequence Synthetic polynucleotide
caggtgcagctgctggaatctggcggcggactggtgcagcctggcggcagcctgagactgagctgcgccgccagcggcttcaccttcagcagctacaccatgagctgggtccggcaggctcctggacaggccctggaatggatgggcaccatcagcagcggcggcacctacacctactaccccgacagcgtgaagggccggttcaccatcagccgggacaacgccaagaacagcctgtacctgcagatgaacagcctgagagccgaggacacagccgtgtactactgcgccagagaggccatcttcacctactggggcagaggcaccctggtcacaagcagcggaggcggaggaagtggagggggaggatcaggcggcggaggcagcgatatccagctgacccagagccctagcagcctgagcgccagcgtgggcgacagagtgaccatcacatgcaaggccagccaggacatcaacaactacctgagctggtatcagcagaagcccggccaggcccccagactgctgatctaccgggccaacagactggtggacggcgtgcccgatagattcagcggcagcggctacggcaccgacttcaccctgaccatcaacaacatcgagtccgaggacgccgcctactacttctgcctgaagtacgacgtgttcccctacaccttcggccagggcaccaaggtggagatcaag

SEQ ID NO: 40 - protein, 15 aa, Artificial Sequence
  Description of Artificial Sequence Synthetic peptide
EPKSCDKTHTCPPCP

SEQ ID NO: 41 - DNA, 45 bp, Artificial Sequence
  Description of Artificial Sequence Synthetic oligonucleotide
gagcccaaatcttgtgacaaaactcacacatgcccaccgtgcccg

SEQ ID NO: 42 - DNA, 60 bp, Artificial Sequence
  Description of Artificial Sequence Synthetic oligonucleotide
gatctcgagcccaaatcttgtgacaaaactcacacatgcccaccgtgcccggatcccaag

SEQ ID NO: 43 - DNA, 204 bp, Artificial Sequence
  Description of Artificial Sequence Synthetic polynucleotide
ttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtcc

SEQ ID NO: 44 - DNA, 336 bp, Artificial Sequence
  Description of Artificial Sequence Synthetic polynucleotide
agagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 45 - protein, 18 aa, Thosea asigna virus
EGRGSLLTCGDVEENPGP

SEQ ID NO: 46 - DNA, 54 bp, Thosea asigna virus
gagggcagaggcagcctgctgacatgtggcgacgtggaagagaacccaggcccc

SEQ ID NO: 47 - protein, 333 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 48 - DNA, 999 bp, Artificial Sequence
  Description of Artificial Sequence Synthetic polynucleotide
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 49 - protein, 30 aa, Artificial Sequence
  Description of Artificial Sequence Synthetic polypeptide
CDIYIWAPLAGTCGVLLLSLVITLYCNHRN

SEQ ID NO: 50 - DNA, 90 bp, Artificial Sequence
  Description of Artificial Sequence Synthetic oligonucleotide
tgcgacatctacatctgggcccctctggccggcacatgtggcgtgctgctgctgagcctcgtgatcaccctgtactgcaaccaccggaac

SEQ ID NO: 51 - protein, 31 aa, Artificial Sequence
  Description of Artificial Sequence Synthetic polypeptide
QPMALIVLGGVAGLLLFIGLGIFFCVRCRHR

SEQ ID NO: 52 - DNA, 93 bp, Artificial Sequence
  Description of Artificial Sequence Synthetic oligonucleotide
cagccaatggccctgattgtgctggggggcgtcgccggcctcctgcttttcattgggctaggcatcttcttctgtgtcaggtgccggcaccga

SEQ ID NO: 53 - protein, 21 aa, Artificial Sequence
  Description of Artificial Sequence Synthetic peptide
GSGEGRGSLLTCGDVEENPGP

SEQ ID NO: 54 - protein, 63 aa, Artificial Sequence
  Description of Artificial Sequence Synthetic polypeptide
GSGSRVTELLYRMKRAETYCPRPLLAIHPTEARHKQKIVAPVKQLLNFDLLKLAGDVESNPGP

SEQ ID NO: 55 - protein, 19 aa, Amphimedon queenslandica
LLCFLLLLLSGDVELNPGP

SEQ ID NO: 56 - protein, 20 aa, Amphimedon queenslandica
HHFMFLLLLLAGDIELNPGP

SEQ ID NO: 57 - protein, 20 aa, Saccoglossus kowalevskii
WFLVLLSFILSGDIEVNPGP

SEQ ID NO: 58 - protein, 20 aa, Branchiostoma floridae
KNCAMYMLLLSGDVETNPGP

SEQ ID NO: 59 - protein, 20 aa, Branchiostoma floridae
MVISQLMLKLAGDVEENPGP

SEQ ID NO: 60 - protein, 22 aa, Artificial Sequence
  Description of Artificial Sequence Synthetic peptide
GSGATNFSLLKQAGDVEENPGP

SEQ ID NO: 61 - protein, 23 aa, Artificial Sequence
  Description of Artificial Sequence Synthetic peptide
GSGQCTNYALLKLAGDVESNPGP

SEQ ID NO: 62 - protein, 8 aa, Unknown
  Description of Unknown 2A consensus sequence
  MOD_RES (2)..(2): Any amino acid
  MOD_RES (4)..(4): Any amino acid
DXEXNPGP

SEQ ID NO: 63 - DNA, 23 bp, Artificial Sequence
  Description of Artificial Sequence Synthetic oligonucleotide
  modified_base (21)..(21): a, c, t, or g
cctgcatgatgcgcggcccangg

SEQ ID NO: 64 - DNA, 23 bp, Artificial Sequence
  Description of Artificial Sequence Synthetic oligonucleotide
  modified_base (21)..(21): a, c, t, or g
caagtagtcggggatgtcggngg

SEQ ID NO: 65 - DNA, 23 bp, Artificial Sequence
  Description of Artificial Sequence Synthetic oligonucleotide
  modified_base (21)..(21): a, c, t, or g
agtagtcggggatgtcggcgngg

SEQ ID NO: 66 - protein, 9 aa, Artificial Sequence
  Description of Artificial Sequence Synthetic peptide
RSKRSRLLH

SEQ ID NO: 67 - DNA, 27 bp, Artificial Sequence
  Description of Artificial Sequence Synthetic oligonucleotide
aggagtaagaggagcaggctcctgcac

SEQ ID NO: 68 - DNA, 23 bp, Artificial Sequence
  Description of Artificial Sequence Synthetic oligonucleotide
  modified_base (21)..(21): a, c, t, or g
gcatgatgcgcggcccaaggngg

SEQ ID NO: 69 - DNA, 57 bp, Artificial Sequence
  Description of Artificial Sequence Synthetic primer
cactctttccctacacgacgctcttccgatcttcccgatgaccctgtcttcccgtgc

SEQ ID NO: 70 - DNA, 59 bp, Artificial Sequence
  Description of Artificial Sequence Synthetic primer
gtgactggagttcagacgtgtgctcttccgatctaggtagaagccattagtgagctggc

SEQ ID NO: 71 - DNA, 32 bp, Artificial Sequence
  Description of Artificial Sequence Synthetic oligonucleotide
ctcctgcatgatgcgcggcccaaggagggaga

(19) United States
(12) Patent Application Publication
(10) Pub. No.: US 2022/0226379 A1
(43) Pub. Date: Jul. 21, 2022

(54) DNMT3A KNOCK-OUT STAT5 ACTIVATED GENETICALLY ENGINEERED T-CELLS

(71) Applicant: ST. JUDE CHILDREN'S RESEARCH HOSPITAL, INC., Memphis, TN (US)

(72) Inventors: Stephen GOTTSCHALK, Germantown, TN (US); Giedre KRENCIUTE, Memphis, TN (US); Christopher PETERSEN, Memphis, TN (US); Benjamin YOUNGBLOOD, Memphis, TN (US)

(73) Assignee: ST. JUDE CHILDREN'S RESEARCH HOSPITAL, INC., Memphis, TN (US)

(21) Appl. No.: 17/602,524

(22) PCT Filed: Apr. 8, 2020
(86) PCT No.: PCT/US20/27280

§ 371 Date: Oct. 8, 2021

Related U.S. Application Data
(60) Provisional application No. 62/831,431, filed on Apr. 9, 2019.

(51) Int. Cl. A61K 35/17; A61P 35/02; A61K 35/545; C12N 9/10

(52) U.S. Cl. CPC A61K 35/17; A61P 35/02; C12Y 201/01037; C12N 9/1007; C12N 2510/00; A61K 35/545

(57) ABSTRACT

The application provides modified immune effector cells wherein the DNA (cytosine-5)-methyltransferase 3A (DNMT3A)-mediated de novo DNA methylation of the cell genome is inhibited, and STAT5 signaling pathway is activated. The application also provides related pharmaceutical compositions and the methods for generating such modified immune effector cells. The application further provides uses of such modified immune effector cells for treating diseases such as cancers, infectious diseases and autoimmune diseases.

### CROSS-REFERENCE TO RELATED APPLICATIONS

This application claims priority to U.S. Provisional Application No. 62/831,431, filed Apr. 9, 2019, the disclosure of which is herein incorporated by reference in its entirety.

### STATEMENT REGARDING FEDERALLY SPONSORED RESEARCH

This invention was made with government support under grant number AI114442 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 3, 2020, is named 243734_000130_SL.txt and is 73,022 bytes in size.

### FIELD

The application relates to modified immune effector cells with enhanced immune cell function, as well as related pharmaceutical compositions. The application further relates to methods for generating the modified immune effector cell and methods for using the modified immune effector cell for treatment of diseases (e.g., adoptive cell therapy).

### BACKGROUND

Cellular immunotherapy with adoptively transferred chimeric antigen receptor (CAR)—modified T cells is an attractive approach to improve the outcomes for patients with cancer. However, T cell therapy for solid tumors has shown so far limited antitumor activity in early phase clinical studies. Even for the most successful CAR T cell therapy (1), CD19-CAR T cell therapy for CD19+ acute lymphoblastic leukemia (ALL), only 50% of patients have responses that last more than one year (11). Complete responses are much lower for CD19+ chronic lymphocytic leukemia (CLL) (12), and only few long-term survivors have been reported for CAR T cell therapies targeting solid tumor or brain tumor antigens such as HER2, mesothelin, CECAM5, GD2, EGFRvIII, and IL13Rα2 (13-18). There exists a need in the art for developing improved antigen-specific T cell therapy. This need can be met with a modified immune effector cell with enhanced effector cell function as disclosed herein.

### SUMMARY OF THE INVENTION

As specified in the Background section above, there is a great need in the art for modified immune effector cells with enhanced immune cell function (e.g., proliferation, antitumor activity) for use in cell therapy for cancer and other disease (e.g., infectious or autoimmune diseases). The present application addresses these and other needs.

In one aspect provided herein is a modified immune effector cell, wherein (i) DNA (cytosine-5)-methyltransferase 3A (DNMT3A)-mediated de novo DNA methylation of the cell genome is inhibited, and (ii) STAT5 signaling pathway is activated.

In some embodiments, the enzymatic activity of the DNMT3A protein is inhibited in the cell. In some embodiments, the enzymatic activity of the DNMT3A protein is inhibited by exposing the cell to a DNMT3A active site inhibitor. In some embodiments, the DNMT3A gene is mutated in DNMT3A catalytic domain so that the enzymatic activity of the DNMT3A protein is inhibited. In some embodiments, the level of functional DNMT3A protein in the cell is decreased by 50% or more. In some embodiments, DNMT3A gene is deleted or defective so that no detectable functional DNMT3A protein is produced.

In some embodiments, the immune effector cell is a T cell. In some embodiments, the T cell is selected from a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg). In some embodiments, the immune effector cell is a natural killer (NK) cell. In some embodiments, the immune effector cell is a stem cell that is capable of differentiating into an immune cell. In some embodiments, the stem cell is an induced pluripotent stem cell (iPSC).

In some embodiments, the STAT5 signaling pathway is activated by a signaling molecule. In some embodiments, the signaling molecule is a common gamma chain cytokine. In some embodiments, the cytokine is IL-15, IL-7, IL-2, IL-4, IL-9, or IL-21. In some embodiments, the STAT5 signaling pathway is activated by modifying the immune effector cell to express a constitutively active cytokine receptor or a switch receptor. In some embodiments, the constitutively active cytokine receptor is a constitutively active IL7 receptor (C7R). In some embodiments, the switch receptor is an IL-4/IL-7 receptor or an IL-4/IL-2 receptor.

In some embodiments, the cell further comprises at least one surface molecule capable of binding specifically to an antigen. In some embodiments, the antigen is selected from a tumor antigen, a viral antigen, a bacterial antigen, a fungal antigen, a parasite antigen, a prion antigen, and an antigen associated with an inflammation or an autoimmune disease. In some embodiments, the tumor antigen is human epidermal growth factor receptor 2 (HER2), IL13Rα2, or erythropoietin-producing human hepatocellular receptor A2 (EphA2).

In some embodiments, the cell further comprises a chimeric antigen receptor (CAR), an antigen specific T-cell receptor, or a bispecific antibody.

In some embodiments, the cell further comprises a chimeric antigen receptor (CAR). In some embodiments, the CAR comprises (i) an extracellular antigen-binding domain and (ii) a transmembrane domain. In some embodiments, the CAR further comprises a cytoplasmic domain.

In some embodiments, the extracellular antigen-binding domain comprises an antibody or an antibody fragment. In some embodiments, the extracellular antigen-binding domain comprises an scFv capable of binding to human epidermal growth factor receptor 2 (HER2). In some embodiments, the scFv capable of binding to HER2 comprises the amino acid sequence of SEQ ID NO: 17. In some embodiments, the extracellular antigen binding domain comprises an scFv capable of binding to IL13Rα2. In some embodiments, the scFv capable of binding to IL13Rα2 comprises the amino acid sequence of SEQ ID NO: 29. In some embodiments, the extracellular antigen binding domain comprises an scFv capable of binding to erythropoietin-producing human hepatocellular receptor A2 (EphA2). In some embodiments, the scFv capable of binding to EphA2 comprises the amino acid sequence of SEQ ID NO: 38. In some embodiments, the extracellular antigen-binding domain further comprises a leader sequence. In some embodiments, the leader sequence comprises the amino acid sequence of SEQ ID NO: 15.

In some embodiments, the transmembrane domain is derived from CD3ζ, CD28, CD4, or CD8α. In some embodiments, the transmembrane domain is derived from CD3ζ and comprises the amino acid sequence SEQ ID NO: 23. In some embodiments, the transmembrane domain is derived from CD28 and comprises the amino acid sequence SEQ ID NO: 31. In some embodiments, the transmembrane domain is derived from CD8α and comprises the amino acid sequence SEQ ID NO: 49. In some embodiments, the transmembrane domain is derived from CD4 and comprises the amino acid sequence SEQ ID NO: 51.

In some embodiments, the CAR further comprises a linker domain between the extracellular antigen-binding domain and the transmembrane domain. In some embodiments, the linker domain comprises a hinge region. In some embodiments, the hinge region comprises the amino acid sequence SEQ ID NO: 19. In some embodiments, the hinge region comprises the amino acid sequence SEQ ID NO: 40. In some embodiments, the linker domain comprises the amino acid sequence SEQ ID NO: 21.

In some embodiments, the CAR cytoplasmic domain comprises one or more lymphocyte activation domains. In some embodiments, the lymphocyte activation domain is derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, or MyD88. In some embodiments, the lymphocyte activation domain is derived from CD3ζ and comprises the amino acid sequence SEQ ID NO: 25. In some embodiments, the CAR cytoplasmic domain comprises one or more co-stimulatory domains. In some embodiments, the co-stimulatory domain is derived from CD28 and comprises the amino acid sequence SEQ ID NO: 33.

In some embodiments, the immune effector cell has been activated and/or expanded ex vivo.

In some embodiments, the immune effector cell is an allogeneic cell. In some embodiments, the immune effector cell is an autologous cell. In some embodiments, the immune effector cell is isolated from a subject having a disease. In some embodiments, the disease is a cancer, an infectious disease, an inflammatory disorder, or an autoimmune disease. In some embodiments, the cancer is a cancer expressing HER2, IL13Rα2, or EphA2. In some embodiments, the cancer is a HER2-positive breast cancer. In some embodiments, the cancer is an IL13Rα2-positive glioblastoma. In some embodiments, the immune effector cell is derived from a blood, marrow, tissue, or a tumor sample.

In another aspect provided herein is a pharmaceutical composition comprising the modified immune effector cell described herein and a pharmaceutically acceptable carrier and/or excipient.

In another aspect provided herein is a method for generating the modified immune effector cell described herein. The method includes deleting or modifying a DNMT3A gene or gene product in the cell so that the DNMT3A-mediated de novo DNA methylation of the cell genome is inhibited.

In some embodiments, the DNMT3A gene in the immune effector cell is deleted or modified as a result of an activity of a site-specific nuclease. In some embodiments, the site-specific nuclease is an RNA-guided endonuclease. In some embodiments, the RNA-guided endonuclease is a Cas9 protein, Cpf1 (Cas12a) protein, C2c1 protein, C2c3 protein, or C2c2 protein. In some embodiments, the RNA-guided endonuclease is a Cas9 protein. In some embodiments, the Cas9 protein is programmed with a guide RNA (gRNA) that comprises a nucleotide sequence encoded by SEQ ID NO: 63, or SEQ ID NO: 68. In other embodiments, the site-specific nuclease is a zinc finger nuclease, a TALEN nuclease, or mega-TALEN nuclease.

In some embodiments, the DNMT3A gene product in the immune effector cell is deleted or modified as a result of an activity of an RNA interference (RNAi) molecule or an antisense oligonucleotide. In some embodiments, the RNAi molecule is a small interfering RNA (siRNA) or a small hairpin RNA (shRNA).

In some embodiments, the site-specific nuclease or the RNAi molecule or the antisense oligonucleotide is introduced into the immune effector cell via a viral vector, a non-viral vector or a physical means.

In some embodiments, the method further includes activating the STAT5 signaling pathway in the immune effector cell by a signaling molecule. In some embodiments, the signaling molecule is a common gamma chain cytokine. In some embodiments, the cytokine is IL-15, IL-7, IL-2, IL-4, IL-9, or IL-21. In some embodiments, the STAT5 signaling pathway is activated by modifying the immune effector cell to express a constitutively active cytokine receptor or a switch receptor. In some embodiments, the constitutively active cytokine receptor is a constitutively active IL7 receptor (C7R). In some embodiments, the switch receptor is an IL-4/IL-7 receptor or an IL-4/IL-2 receptor. In some embodiments, the immune effector cell is contacted with an effective amount of the signaling molecule or a carrier comprising the signaling molecule. In some embodiments, the carrier is a nanoparticle. In some embodiments, the immune effector cell is contacted with the signaling molecule more than once. In some embodiments, the signaling molecule is expressed in the immune effector cell. In some embodiments, the signaling molecule is expressed from a transgene introduced into the immune effector cell. In some embodiments, the signaling molecule-expressing transgene is introduced into the immune effector cell using a viral vector, a non-viral vector or a physical means.

In some embodiments, the CAR is expressed from a transgene introduced into the immune effector cell. In some embodiments, the CAR-expressing transgene is introduced into the immune effector cell using a viral vector, a non-viral vector or a physical means.

In various embodiments, the viral vector is a retroviral vector, an adenoviral vector, an adeno-associated viral (AAV) vector, a herpes viral vector, or a baculoviral vector. In some embodiments, the retroviral vector is a lentiviral vector.

In various embodiments, the non-viral vector is a transposon. In some embodiments, the transposon is a sleeping beauty transposon or PiggyBac transposon.

In various embodiments, the physical means is electroporation, microinjection, magnetofection, ultrasound, a ballistic or hydrodynamic method, or a combination thereof.

In various embodiments, the modified immune effector cell is activated and/or expanded ex vivo.

In another aspect provided herein is a method of treating a disease in a subject in need thereof comprising administering to the subject an effective amount of the modified immune effector cells or the pharmaceutical composition described herein. In some embodiments, the modified immune effector cell is an autologous cell. In some embodiments, the modified immune effector cell is an allogeneic cell. In some embodiments, the disease is a cancer, an infectious disease, an inflammatory disorder, or an autoimmune disease. In some embodiments, the cancer is a solid tumor. In some embodiments, the cancer is breast, prostate, urinary bladder, skin, lung, ovary, sarcoma, or brain cancer. In some embodiments, the cancer is a cancer expressing HER2, IL13Rα2, or EphA2. In some embodiments, the cancer is a HER2-positive breast cancer. In some embodiments, the cancer is an IL13Rα2-positive glioblastoma.

In some embodiments, the treatment method includes: a) isolating an immune effector cell from the subject or a donor; b) modifying a DNMT3A gene or gene product in the immune effector cell such that the DNMT3A-mediated de novo DNA methylation of the cell genome is inhibited; c) activating the STAT5 signaling pathway in the immune effector cell by either stimulating the immune effector cell with a signaling molecule or genetically modifying the immune effector cell to express a signaling molecule; and d) introducing the modified immune effector cell into the subject before or after step (c).

In some embodiments, stimulating the immune effector cell with a signaling molecule is carried out by mixing the immune effector cell with nanoparticles containing the signaling molecule. In some embodiments, the method further includes genetically modifying the immune effector cell to express a chimeric antigen receptor (CAR) that is capable of binding specifically to an antigen. In some embodiments, the subject is a human.

In another aspect provided herein is a method of enhancing an antitumor activity of a T cell, comprising: a) modifying a DNMT3A gene or gene product in the cell so that the DNMT3A-mediated de novo DNA methylation of the cell genome is inhibited; and b) activating the STAT5 signaling pathway in the cell by either stimulating the cell with a signaling molecule or genetically modifying the cell to express a signaling molecule. In some embodiments, the signaling molecule is IL-15, IL-7, IL-2, IL-4, IL-9, or IL-21. In some embodiments, the method further includes genetically modifying the T cell to express a chimeric antigen receptor (CAR) that is capable of binding to an antigen specific for the tumor. In some embodiments, the method includes activation and/or expansion of the T cell ex vivo.

These and other aspects of the present invention will be apparent to those of ordinary skill in the art in the following description, claims and drawings.

### BRIEF DESCRIPTION OF THE DRAWINGS

U.S. Provisional Application No. 62/831,431 contains copies of several of the drawing(s) below in color, which can be provided by the United States Patent and Trademark Office upon request and payment of the necessary fee.

FIGS. 1A-1G illustrate the procedure for the generation and stimulation of gene edited CAR T cells. (FIG. 1A) Generation of gene edited CAR T cells. The gene edited CAR T cells were generated by first transducing activated T cells with retroviral vectors encoding CARs, and then electroporated with Cas9/DNMT3A-specific sgRNA complexes (referred to as RNP complexes). (FIG. 1B) Repeat stimulation experiment protocol. 1×10⁶ CART cells were co-cultured with 0.5×10⁶ tumor cells (U373 cells from ATCC) at a 2:1 E:T ratio in the presence or absence of IL-15 (stimulation 1). 7 days later, T cells were harvested, counted and 1×10⁶ T cells were re-plated on fresh 0.5×10⁶ tumor cells in the presence or absence of IL-15 (stimulation 2). This procedure was repeated weekly until the T cells stopped expanding and/or started dying. (FIG. 1C) Scheme of EphA2, HER2 and IL13Rα2-specific CAR retroviral vectors. SSR: short spacer region (40, 41). (FIG. 1D) Schematic of the Cas9/gRNA target sites for DNMT3A. The gRNA target sequence for guide 2 (g2) and guide 3 (g3) are underlined, and the PAM sequences are boxed. Numbers in the filled boxes indicate DNMT3A exons. The target site sequence shown for both g2 and g3 is SEQ ID NO: 71. (FIG. 1E) Targeted next-generation sequencing (NGS) assay was used to validate cutting efficacy for g2 and g3. (FIG. 1F) Western Blot analysis of DNMT3A expression in control (abbreviated as Ctrl or Co) and DNMT3A knockout (abbreviated as k/o or KO) (g2) in IL13Rα2- and EphA2-CAR T cells. GAPDH was used as a loading control; two independent donors are shown. (FIG. 1G) Western Blot analysis of DNMT3A expression in control knockout and DNMT3A knockout HER2-specific 1^{st} and 2^{nd} generation CAR T cells. Two guide RNAs targeting DNMT3A were tested.

FIGS. 2A-2D show the proliferation of gene edited HER2-CAR T cells in the absence of IL-15. Gene edited HER2-CAR T cells were generated expressing 1^{st} generation and 2^{nd} HER2-specific chimeric antigen receptors (HER2-CARs). As controls, HER2-CAR T cells were electroporated with Cas9 sgRNA complexes specific for an irrelevant gene (mCherry g17; HER2-CAR.Ctrl.k/o-T cells). To evaluate the ability of HER2-CAR.DNMT3A.k/o-T cells to proliferate after repeat stimulations, repeat stimulation experiment was performed as shown in FIG. 1B. This experiment was conducted independently for 2 donors. (FIGS. 2A-2B) Expansion of gene edited T cells from 2 donors expressing a 1^{st} generation HER2-CAR (HER2-CAR.ζ). (FIGS. 2C-2D) Expansion of gene edited T cells from 2 donors expressing a 2^{nd} generation HER2-CAR (HER2-CAR.CD28.ζ).

FIGS. 3A-3D show the proliferation of gene edited HER2-CAR T cells in the presence of IL-15. (FIGS. 3A-3B) Expansion of gene edited T cells from 2 donors expressing a 1^{st} generation HER2-CAR (HER2-CAR.ζ). (FIGS. 3C-3D) Expansion of gene edited T cells from 2 donors expressing a 2^{nd} generation HER2-CAR (HER2-CAR.CD28.ζ).

FIGS. 4A-4D show the antitumor ability of the gene edited HER2-CAR T cells in the presence of IL15. (FIG. 4A) Tumor cell survival in the presence of gene edited T cells expressing a 1^{st} generation HER2-CAR (HER2-CAR.ζ) after the 1^{st} stimulation with IL15. (FIG. 4B) Tumor cell survival in the presence of gene edited T cells expressing a 2^{nd} generation HER2-CAR (HER2-CAR.CD28.ζ) after the 1^{st} stimulation with IL15. (FIG. 4C) Tumor cell survival in the presence of gene edited T cells expressing a 1^{st} generation HER2-CAR (HER2-CAR.ζ) after 4^{th} stimulation with IL15. (FIG. 4D) Tumor cell survival in the presence of gene edited T cells expressing a 2^{nd} generation HER2-CAR (HER2-CAR.CD28.ζ) after the 4^{th} stimulation with IL15.

FIGS. 5A-5D show the proliferation and antitumor activity of gene edited IL13Rα2-CAR T cells. Gene edited IL13Rα2-CAR T cells were generated expressing a 2^{nd} generation IL13Rα2-CAR (IL13Rα2-CAR.CD28.ζ). As controls, IL13Rα2-CAR T cells were electroporated with Cas9 sgRNA complexes specific for an irrelevant gene (mCherry g17; IL13Rα2-CAR.Ctrl.k/o-T cells). The proliferation and antitumor activity of IL13Rα2-CAR.DNMT3A.k/o-T cells were evaluated similarly as described for the HER2-CAR. DNMT3A.k/o-T cells above. (FIG. 5A) Proliferation of IL13Rα2-CAR T cells in the absence of IL-15. (FIG. 5B) Proliferation of IL13Rα2-CAR T cells in the presence of IL-15. (FIG. 5C) Tumor cell survival in the presence of gene edited IL13Rα2-CAR T cells after the 1^{st} stimulation with IL15. (FIG. 5D) Tumor cell survival in the presence of gene edited IL13Rα2-CAR T cells after the 4^{th} stimulation with IL15.

FIGS. 6A-6D demonstrate that DNMT3A deletion enhances CAR T cell expansion during repeat stimulation in vitro. (FIG. 6A) Ctrl.k/o and DNMT3A.k/o CAR T cell expansion after weekly stimulations with U373 cells for HER2-CAR.ζ (n=3), HER2-CAR.CD28.ζ (n=5), IL13Rα2-CAR.CD28.ζ (n=4), and EphA2-CAR.CD28.ζ (n=5) T cells. (FIG. 6B) Expansion of DNMT3A.k/o relative to Ctrl.k/o CAR T cells at each of the first four stimulations (n=17, unpaired t-test). (FIGS. 6C, 6D) After the 4^{th} stimulation with tumor cells, DNMT3A.k/o HER-CAR.ζ or HER2-CAR.CD28.ζ T cells were sorted and plated with or without antigen (U373 tumor cells) in the presence or absence of IL-15. After 7 days dead, dying, and live T cells were determined by flow cytometry. (FIG. 6C) Representative flow plots. (FIG. 6D) Summary data for all CARs, donors, and conditions evaluated (mean+SEM; HER2-CAR.CD28.ζ: n=9, HER2-CAR.ζ: n=4; 2-way ANOVA with Tukey's test for multiple comparisons). ns=not significant, p<0.05: *, p<0.01: **, p<0.001: ***, p<0.0001: ****.

FIG. 7 demonstrates that silencing DNMT3A with different guide RNAs shows the same improved effector function. An alternative sgRNA, guide 3, was used to silence DNMT3A in HER2-CAR.CD28.ζ (filled circles) and HER2-CAR.ζ (filled squares) T cells. A repeated stimulation experiment was set up as described in FIG. 1B with HER2-CAR.CD28.ζ used as Ctrl.k/o cells (blank circles). The assay was carried out until CAR T ceased to proliferate at which point cocultures were terminated.

FIGS. 8A-8B demonstrate that deletion of DNMT3A in CAR T cells enhances performance in a repeat stimulation assay against LM7. Ctrl and DNMT3A knockout EphA2-CAR.CD28.ζ, HER2-CAR.ζ, and HER2-CAR.CD28.ζ T cells were used in a repeated stimulation assay as described in FIG. 1B against the osteosarcoma cell line LM7. (FIG. 8A) Fold T cell expansion for each donor. (FIG. 8B) Expansion of DNMT3A.k/o relative to Ctrl.k/o CAR T cells at each of the first four stimulations (n=10, unpaired t-test, ns=not significant, p<0.05: *).

FIGS. 9A-9C show sustained cytokine secretion and cytolytic activity of DNMT3A.k/o CAR T cells after repeat antigen stimulation. To measure Th1 and Th2 cytokine production, Ctrl and DNMT3A knockout CAR T cells were stimulated as described in FIG. 1B. Cell culture supernatants were harvested 24 hours after each stimulation, and a multiplex assay (EMD Millipore) was used to determine cytokine production. (FIG. 9A) Summary of Th1 and Th2 cytokine production and fold-change in cytokine production of Ctrl vs DNMT3A knockout CAR T cells after the first stimulation with tumor cells are shown (mean+SEM; n=3/CAR construct; one-way ANOVA with Dunnett's multiple comparisons test). (FIG. 9B) Cytokine production as described above after the 4^{th} stimulation with fresh tumor cells (mean+SEM; n=3/CAR construct; one-way ANOVA with Dunnett's multiple comparisons test; #: set to 100-fold (actual value: 174-fold). (FIG. 9C) A 24-hour MTS assay was used to assess the cytolytic activity of Ctrl and DNMT3A knockout HER-CAR.ζ, HER2-CAR.CD28.ζ and IL13Rα2-CAR.CD28.ζ T cells at their first (1^{st} stim) or fourth (4^{th} stim) exposure to tumor cells in the repeat stimulation assay described in FIG. 1B (mean+SEM, n=3-5; paired t-test). All statistical tests compared Ctrl to DNMT3A knockout; p<0.05: *, p<0.01: **, p<0.001: ***, p<0.0001: ****.

FIGS. 10A-10B show the results of whole-genome DNA methylation profiling of gene edited CAR T cells. (FIG. 10A) DNA methylation-based T cell multipotency index of gene edited CAR T cells. The DNA methylation-based T cell multipotency index was used to compare the epigenetic state of DNMT3A.k/o and Ctrl.k/o CAR T cells (WT CAR T cells). (FIG. 10B) Methylation status of the transcription factor Tcf1 (encoded by the gene TCF7) promotor and DNMT3A promoter. (FIG. 10C) Experimental setup with a summary graph of Ctrl.k/o vs DNMT3A.k/o CAR T cell expansion with subsequent data analysis from week 4.

FIGS. 11A-11B demonstrate that the expansion of CAR.DNMT3A.k/o-T cells is antigen dependent. (FIG. 11A) Expansion of CAR.DNMT3A.k/o-T cells in the absence of antigen stimulation. (FIG. 11B) Expansion of CAR.DNMT3A.k/o-T cells in the presence of antigen stimulation.

FIG. 12 shows the proliferation of gene edited EphA2-CAR T cells in the presence of IL-15.

FIGS. 13A-13B demonstrate that EphA2-specific DNMT3A.k/o CAR T cells showed better anti-tumor activity in vivo. (FIG. 13A) Scheme for the in vivo testing of gene edited CAR T cells using an intraperitoneal LM7 xenograft model. 8-week old female NSG mice are injected intraperitoneally (i.p.) with 10⁶ LM7 tumor cells, which were genetically modified to express firefly luciferase (ffluc) to enable non-invasive bioluminescence imaging to track tumor cells. On day 7, mice receive a single i.p. injection of 10³ or 10⁴ DNMT3A.k/o or Ctrl.k/o CAR T cells. Each treatment group consisted of 5 mice. Tumor burden was monitored by bioluminescence imaging using an in vivo imaging system (IVIS) Spectrum imager. (FIG. 13B) Tumor growth in mice treated with DNMT3A.k/o or Ctrl.k/o CAR T cells.

FIG. 14 outlines the scheme for in vivo testing of gene edited CAR T cells using an orthotopic U373 glioma xenograft mouse model. On Day 0, U373.eGFP.ffLuc cells are injected stereotactically into brains of NSG mice. On Day 7, mice receive a single intracranial (i.c.) injection of 10³ or 10⁴ DNMT3A.k/o or Ctrl.k/o CAR T cells. Each treatment group consists of 5 mice. Tumor burden is monitored by bioluminescence imaging using an IVIS Spectrum imager.

FIGS. 15A-15I show that DNMT3A deletion enhances CAR T cell antitumor activity in vivo. (FIG. 15A) Scheme of intraperitoneal (i.p.) LM7 model. NSG mice were injected i.p. with 1×10⁶ LM7-ffLuc tumor cells on Day 0, and 7 days later received a single i.p. dose of 3×10⁵ Ctrl.k/o or DNMT3A.k/o EphA2-CAR T cells that were previously stimulated with recombinant human EphA2 protein (Act EphA2 CARTs). (FIG. 15B) Quantitative bioluminescence imaging (total flux). (FIG. 15C) Kaplan Meier survival curve (n=5 mice per group, log-rank (Mantel-Cox) test, p<0.05: *). (FIG. 15D) Scheme of intravenous (i.v.) LM7 model. NSG mice were injected i.v. with 2×10⁶ LM7-ffLuc tumor cells, and 28 days later received a single i.v. dose of 1×10⁶ Ctrl.k/o or DNMT3A.k/o HER2-CAR.ζ T cells or PBS. (FIG. 15E) Quantitative bioluminescence imaging (total flux). (FIG. 15F) Kaplan Meier survival curve (n=5 mice per group, log-rank (Mantel-Cox) test, p<0.01: **). (FIG. 15G) Scheme of intracranial U373 model. NSG mice were injected with 5×10⁴ U373-ffLuc tumor cells and 7 days later received a single intratumoral dose of 2×10⁶ Ctrl.k/o or DNMT3A.k/o IL13Rα2-CAR/IL15 T cells. (FIG. 15H) Quantitative bioluminescence imaging (total flux). (FIG. 15I) After initial progression (defined as a >4-fold increase in Total Flux between consecutive measurements), tumor growth rates were calculated by dividing consecutive Total Flux data points. Slow tumor growth is defined as <1.5 fold change per week. Plotted are the number of measurements per mouse that were <1.5 (two-way ANOVA, p<0.05: *).

FIGS. 16A-16C demonstrate that DNMT3A deletion promotes selective survival of human T cell subsets that have high multipotent differentiation potential. (FIG. 16A) Gene set enrichment analysis (GSEA) of DNMT3A-targeted transcription factors among human naïve and memory CD8+ T cell subsets. (FIG. 16B) Schematic representation of the sorting procedure used to isolate naïve/Tscm and differentiated CD8+ T cell subsets with subsequent generation of subsetted DNMT3A.k/o CAR T cells. (FIG. 16C) Expansion of the naïve/Tscm and differentiated CAR T cell subsets in the repeat stimulation assay (FIG. 1B; n=4).

### DETAILED DESCRIPTION

The present invention generally provides modified immune effector cells (e.g., T cells or natural killer (NK) cells), particularly cells with enhanced immune cell function (e.g., proliferation, antitumor activity).

Given that the therapeutic efficacy of CAR T cell approaches has been closely linked to the T cell's in vivo persistence and sustained effector functions (17-19), a critical and universally agreed upon goal for the field is to identify mechanism(s) that restricts CAR T cell survival and function. Several studies have reported that prolonged stimulation of CAR T cells results in a functional exhaustion of their antitumor properties and promotes a rapid contraction in their quantity (7, 8). These hallmarks suggest that CAR T cells have undergone the developmental process of exhaustion; however, a more refined definition for CAR T cell exhaustion is lacking. In model systems that have established the concept of T cell exhaustion, it is now clear that this developmental state is epigenetically reinforced to maintain exhaustion-associated gene expression programs (20-24). Importantly, epigenetic modifications can provide a cell-intrinsic mechanism that enables memory T cells to retain acquired gene expression programs during their antigen-independent homeostasis, indicating that once acquired, exhaustion-associated epigenetic programs can be long-lived (22, 23, 25-32).

The present disclosure shows a surprisingly and unexpectedly discovery that DNMT3A inhibition enhances the effector function and survival of immune effector cells (e.g., CAR T cells). Blocking de novo methylation programming, for example, by deleting DNMT3A, in immune effector cells can preserve their differentiation capacity, allowing for sustained proliferation and effector function in the setting of chronic antigen exposure.

### Definitions

The term “immune effector cell” as used herein refers to a cell that is involved in an immune response, e.g., in the promotion of an immune effector response. Non-limiting examples of immune effector cells include T cells (e.g., αβ T cells and γδ T cells), B cells, natural killer (NK) cells, natural killer T (NKT) cells, mast cells, and myeloid-derived phagocytes. Stem cells, such induced pluripotent stem cells (iPSCs), that are capable of differentiating into immune cells are also included here.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a CD8+ T cell, a CD4+ T cell, a helper T cell or T-helper cell (HTL; CD4+ T cell), a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “αβ T cell receptor (TCR) T cells”, which refer to a population of T cells that possess a TCR composed of α- and β-TCR chains. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+T cells.

The terms “natural killer cell” and “NK cell” are used interchangeable and used synonymously herein. As used herein, NK cell refers to a differentiated lymphocyte with a CD 16+ CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The term “signaling molecule” as used herein, refers to any molecule that is capable of inducing a direct or indirect response in at least one cellular signaling pathway. The response may be stimulatory or inhibitory. One of the cellular signaling pathways may be the STAT5 signaling pathway.

The term “switch receptor” used herein refers to a receptor that is capable of converting a potentially inhibitory signal into a positive signal. Switch receptors are also known as inverted cytokine receptors.

The term “chimeric antigen receptor” or “CAR” as used herein is defined as a cell-surface receptor comprising an extracellular target-binding domain, a transmembrane domain and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention are intended primarily for use with lymphocyte such as T cells and natural killer (NK) cells.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T Cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910, each of which are incorporated by reference in their entirety for all purposes. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass. Also included are “bispecific antibodies”, which refer to antibodies that are capable of binding to two different antigens or different epitopes of the same antigen.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

Host cells of the present invention include T cells and natural killer cells that contain the DNA or RNA sequences encoding the CAR and express the CAR on the cell surface. Host cells may be used for enhancing T cell activity, natural killer cell activity, treatment of cancer, and treatment of autoimmune disease.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

As used herein, the term “derivative” in the context of proteins or polypeptides (e.g., CAR constructs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative of; (d) a polypeptide encoded by nucleic acids can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wis.). Information regarding hybridization conditions (e.g., high, moderate, and typical stringency conditions) have been described, see, e.g., U.S. Patent Application Publication No. US 2005/0048549 (e.g., paragraphs 72-73).

The term “variant” as used herein refers to a modified polypeptide, protein, or polynucleotide that has substantial or significant sequence identity or similarity to a wild type polypeptide, protein, or polynucleotide. The variant may retain the same, or have altered (e.g., improved, reduced or abolished) biological activity relative to the wild type polypeptide, protein, or polynucleotide of which it is a variant. The variant may contain an insertion, a deletion, a substitution of at least one amino acid residue or nucleotide.

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In some embodiments, the vector is a viral vector such as, but not limited to, viral vector is an adenoviral, adeno-associated, alphaviral, herpes, lentiviral, retroviral, baculoviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in antitumor activity (e.g., cancer cell death killing ability), among others apparent from the understanding in the art and the description herein. In some embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In some embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response (reference response) produced by vehicle, a control composition, or the response in a particular cell lineage.

The terms “inhibit” or “inhibition” as used herein refer to reducing a function or activity to an extent sufficient to achieve a desired biological or physiological effect. Inhibition may be complete or partial.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising” a feature, or versions there of (e.g., comprise), embodiments also are contemplated “consisting of” or “consisting essentially of” the feature.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Modified Immune Effector Cells

In one aspect, the invention provides a modified immune effector cell with enhanced immune cell function. In particular, the immune effector cell is modified such that the DNA (cytosine-5)-methyltransferase 3A (DNMT3A)-mediated de novo DNA methylation of the cell genome is inhibited and STAT5 signaling pathway is activated.

In some embodiments, the immune effector cell is a T cell. T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+ CD8+ T cell, or any other subset of T cells. Other illustrative populations of T cells suitable for use in particular embodiments include naive T cells memory T cells, and NKT cells.

In some embodiments, the T cell may be a CD8+ T cell, a CD4+ T cell, a cytotoxic T cell, an αβ T cell receptor (TCR) T cell, a natural killer T (NKT) cell, a γδ T cell, a memory T cell, a T-helper cell, or a regulatory T cell (Treg).

The modification may be applied to all forms of T cell therapies, which include but not limited to therapies with i) T cells that express a chimeric antigen receptor (CAR); ii) T cells that express an endogenous αβ TCR, which is specific for a peptide derived from viral or tumor-associated antigens (including neoantigens); iii) T cells that transgenically express an αβ TCR, which is specific for a peptide derived from viral or tumor-associated antigens (including neoantigens); iv) T cells that transgenically express bispecific antibodies, which recognize viral or tumor-associated antigens (including neoantigens)/or a peptide derived from them and an activating molecule expressed on T cells such as CD3; and/or v) T cells that are generated via stimulation with for examples but not limited to peptides, antigen presenting and/or artificial antigen presenting cells (in vitro sensitized [IVS] T cell therapy). Lastly, T cell therapies in which the therapeutic genes are delivered in vivo are also included (in vivo T cell therapy).

In some embodiments, the immune effector cell is a natural killer (NK) cell. NK cell refers to a differentiated lymphocyte with a CD3− CD16+, CD3− CD56+, CD16+ CD56+ and/or CD57+ TCR− phenotype.

In some embodiments, the immune effector cell is a stem cell that is capable of differentiating into an immune cell. The stem cell may be an induced pluripotent stem cell (iPSC).

DNA (cytosine-5)-methyltransferase 3A (DNMT3A) is an enzyme that catalyzes the addition of methyl groups to cytosine residues of CpG structures in DNA. The enzyme is encoded in humans by the DNMT3A gene. This enzyme is responsible for de novo DNA methylation. Such function may be different from maintenance DNA methylation which ensures the fidelity of replication of inherited epigenetic patterns. The DNMT3A-mediated de novo DNA methylation is critical in DNA imprinting and modulation of gene expression.

In some embodiments, the enzymatic activity of the DNMT3A protein is inhibited in the cell. The enzymatic activity of the DNMT3A protein may be inhibited by exposing the cell to a DNMT3A active site inhibitor. Although not wishing to be bound by theory, the methyl-transfer reaction carried out by a DNA methyltransferase is typically initiated by nucleophilic attack from a catalytic cysteine in the active site. The catalytic cysteine is highly conserved among cytosine methyltransferases. When the catalytic cysteine is mutated or blocked the enzymatic activity of the DNMT3A protein can be inhibited, although binding may still occur. The catalytic cysteine of human DNMT3A has been identified to be C710 (Zhang, Z. M. et al., Nature. 2018; 554(7692): 387-391, which is incorporated herein by reference in its entirety for all purposes). Examples of DNMT3A active site inhibitors that may be used in the present invention include 5-azacytidine, Decitabine, Zebularine, 5-fluoro-2′-deoxycytidine, as well as other cytidine analogs known in the art. A further example of a DNMT3A active site inhibitor includes RG108.

In some embodiments, the DNMT3A gene is mutated in the DNMT3A catalytic domain so that the enzymatic activity of the DNMT3A protein is inhibited. As a non-limiting example, a catalytic cysteine in the catalytic domain may be mutated in a way that the enzymatic reaction can no longer occur.

In some embodiments, the level of functional DNMT3A protein in the cell is decreased by about 50% or more. The level of functional DNMT3A protein in the cell may be decreased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of functional DNMT3A protein in the cell may be decreased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the DNMT3A gene is deleted or defective so that no detectable wild-type DNMT3A protein is produced. The DNMT3A gene may be deleted or become defective using the methods described herein.

In some embodiments, the STAT5 signaling pathway is activated by a signaling molecule. The signaling molecule may be a common gamma chain cytokine. Non-limiting examples of cytokines that may be used in the methods described herein include IL-15, IL-7, IL-2, IL-4, IL-9, and IL-21. The cytokine may be a native or modified cytokine. In some embodiments, the signaling molecule is IL-15. In some embodiments, the signaling molecule is IL-7.

In some embodiments, the STAT5 signaling pathway is activated by modifying the immune effector cell to express a constitutively active cytokine receptor or a switch receptor. Constitutively active cytokine receptors may trigger the activation of a cytokine signaling cascade even in the absence of extracellular cytokine. This may circumvent the need for providing extracellular cytokines to the immune effector cell. A non-limiting example of a constitutively active cytokine receptor is a constitutively active IL7 receptor (C7R). Such constitutively active cytokine receptor may be generated using methods described in Shum T et al. Cancer Discov. 2017; 7(11):1238-1247, which is incorporated herein in its entirety for all purposes.

A switch receptor (also known as inverted cytokine receptor), which is capable of converting a potentially inhibitory signal into a positive signal, is also contemplated by the present invention. Non-limiting examples of switch receptors that may also be used in the methods described herein include an IL4/IL7 receptor and an IL4/IL2 receptor. Such receptors may be generated as described in Bajgain, P. et al., J Immunother Cancer. 2018; 6(1):34 and Wilkie, S. et al., J Biol Chem. 2010; 285(33):25538-44, both of which are incorporated herein by reference in their entirety for all purposes.

In some embodiments, the modified immune effector cell further comprises at least one surface molecule capable of binding specifically to an antigen. The antigen may be a tumor antigen, a viral antigen, a bacterial antigen, a fungal antigen, a parasite antigen, a prion antigen, or an antigen associated with an inflammation or an autoimmune disease.

In some embodiments, the antigen is a tumor antigen. Non-limiting examples of tumor antigens that may be targeted by the modified immune effector cell described herein include human epidermal growth factor receptor 2 (HER2), interleukin-13 receptor subunit alpha-2 (IL-13Ra2), ephrin type-A receptor 2 (EphA2), A kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLEC12A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRCSD), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAXS), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof.

Additional antigens that may be targeted by the extracellular target-binding domain include, but are not limited to, carbonic anhydrase EX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD45, CD74, CD79a, CD80, CD138, colon-specific antigen-p (CSAp), CEA (CEACAM5), CEACAM6, CSAp, EGFR, EGP-I, EGP-2, Ep-CAM, EphA1, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In some embodiments, the tumor antigen targeted by the modified immune effector cell is HER2, IL13Rα2, or EphA2, or a fragment or variant thereof.

In some embodiments, the tumor antigen targeted by the modified immune effector cell is HER2. Human epidermal growth factor receptor 2 (HER2), also referred to as HER2/neu, receptor tyrosine-protein kinase erbB-2, CD340 (cluster of differentiation 340), proto-oncogene Neu, or ERBB2, is a membrane tyrosine kinase and oncogene that is overexpressed in some types of cancer.

In some embodiments, the tumor antigen targeted by the modified immune effector cell is IL13Rα2. Interleukin-13 receptor subunit alpha-2 (IL13Rα2), also referred to as CD213A2 (cluster of differentiation 213A2), is a membrane bound protein that in humans is encoded by the IL13RA2 gene.

In some embodiments, the tumor antigen targeted by the modified immune effector cell is EphA2. Ephrin type-A receptor 2 (EphA2), also referred to as Eck (epithelial cell kinase), Myk2, or Sek2, is a member of the Eph receptor tyrosine kinase family which binds Ephrins A1, 2, 3, 4, and 5.

In some embodiments, the modified immune effector cell further comprises a chimeric antigen receptor (CAR), an antigen specific T-cell receptor, or a bispecific antibody.

In some embodiments, the modified immune effector cell further comprises an antigen specific T-cell receptor. Antigen specific T-cell receptors are T-cell receptors (TCRs) that are specific for recognizing a particular antigen.

In some embodiments, the modified immune effector cell further comprises a bispecific antibody. Bispecific antibodies are antibodies that are capable of binding to two different antigens or different epitopes of the same antigen. For example, the modified immune effector cell may comprise a bispecific antibody that is capable of binding to an molecule on the immune effector cell and is also capable of binding to an antigen on a target cell.

### Chimeric Antigen Receptor (CAR)

In some embodiments, the modified immune effector cell further comprises a chimeric antigen receptor (CAR).

CARs are typically comprised primarily of 1) an antigen-binding moiety, such as a single-chain variable fragment (scFv) derived from an antigen-specific monoclonal antibody, and 2) a lymphocyte activation domain, such as the ζ-chain from the T cell receptor CD3. These two regions are fused together via a transmembrane domain. Upon transduction, the lymphocyte expresses the CAR on its surface, and upon contact and ligation with the target antigen, it signals through the lymphocyte activation domain (e.g., CD3ζ chain) inducing cytotoxicity and cellular activation.

Constructs with only the antigen-specific binding region together with the lymphocyte activation domain are termed first-generation CARs. While activation of lymphocytes through a lymphocyte activation domain such as CD3ζ is sufficient to induce tumor-specific killing, such CARs fail to optimally induce T cell proliferation and survival in vivo. The second-generation CARs added co-stimulatory polypeptides to boost the CAR-induced immune response. For example, the co-stimulating polypeptide CD28 signaling domain was added to the CAR construct. This region generally contains the transmembrane region of the co-stimulatory peptide (in place of the CD3ζ transmembrane domain) with motifs for binding other molecules such as PI3K and Lck. T cells expressing CARs with only CD3ζ vs CARs with both CD3ζ and a co-stimulatory domain (e.g., CD28) demonstrated the CARs expressing both domains achieve greater activity. The most commonly used co-stimulating molecules include CD28 and 4-1BB, which promotes both T cell proliferation and cell survival. The third-generation CAR includes three signaling domains (e.g., CD3ζ, CD28, and 4-1BB), which further improves lymphocyte cell survival and efficacy. Examples of third-generation CARs include CD19 CARs, most notably for the treatment of chronic lymphocytic leukemia (Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365: 725-533, each of which is herein incorporated by reference in their entirety for all purposes). Studies in three patients showed impressive function, expanding more than a 1000-fold in vivo, and resulted in sustained remission in all three patients.

In some embodiments, the CAR expressed by a modified immune effector cell described herein comprises an extracellular antigen-binding domain and a transmembrane domain. In some embodiments, the CAR further comprises a cytoplasmic domain. Each domain is fused in frame.

In some embodiments, the CAR expressed by a modified immune effector cell described herein is a first-generation CAR. In some embodiments, the CAR expressed by a modified immune effector cell described herein is a second-generation CAR.

Extracellular Antigen-Binding Domain of the CAR

The choice of antigen-binding domain depends upon the type and number of antigens that define the surface of a target cell. For example, the antigen-binding domain may be chosen to recognize an antigen that acts as a cell surface marker on target cells associated with a particular disease state. In some embodiments, the CARs can be genetically modified to target a tumor antigen of interest by way of engineering a desired antigen-binding domain that specifically binds to an antigen (e.g., on a cancer cell). Non-limiting examples of cell surface markers that may act as targets for the antigen-binding domain in the CAR include those associated with viral, bacterial and parasitic infections, autoimmune disease, and cancer cells.

In some embodiments, the extracellular antigen-binding domain comprises an antigen-binding polypeptide or functional variant thereof that binds to an antigen. In some embodiments, the antigen-binding polypeptide is an antibody or an antibody fragment that binds to an antigen.

In some embodiments, the antigen-binding polypeptide can be monomeric or multimeric (e.g., homodimeric or heterodimeric), or associated with multiple proteins in a non-covalent complex. In some embodiments, the extracellular target-binding domain may consist of an Ig heavy chain. In some embodiments, the Ig heavy chain can be covalently associated with Ig light chain (e.g., via the hinge and optionally the CH1 region). In some embodiments, the Ig heavy chain may become covalently associated with other Ig heavy/light chain complexes (e.g., by the presence of hinge, CH2, and/or CH3 domains). In the latter case, the heavy/light chain complex that becomes joined to the chimeric construct may constitute an antibody with a specificity distinct from the antibody specificity of the chimeric construct. In some embodiments, the entire chain may be used. In some embodiments, a truncated chain may be used, where all or a part of the CH1, CH2, or CH3 domains may be removed or all or part of the hinge region may be removed. Non-limiting examples of antigen-binding polypeptides include antibodies and antibody fragments such as e.g., murine antibodies, rabbit antibodies, human antibodies, fully humanized antibodies, single chain variable fragments (scFv), single chain antibodies, Fab fragments, F(ab′) fragments, disulfide-linked Fvs (sdFv), intrabodies, minibodies, or diabodies, camelid antibody variable domains and humanized versions, shark antibody variable domains and humanized versions, single domain antibody variable domains, nanobodies (VHHs), and camelized antibody variable domains. In some embodiments, the antigen-binding polypeptide include an scFv.

In some embodiments, the extracellular antigen-binding domain is specific for HER2, or a fragment or variant thereof. In some embodiments, the extracellular antigen-binding domain is specific for IL13Rα2, or a fragment or variant thereof. In some embodiments, the extracellular antigen-binding domain is specific for EphA2, or a fragment or variant thereof.

In a specific embodiment, the extracellular antigen-binding domain comprises an scFv capable of binding to HER2. The scFv capable of binding to HER2 may comprise the amino acid sequence of SEQ ID NO: 17, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 17. In some embodiments, the nucleotide sequence encoding the anti-HER2 scFV comprises the nucleotide sequence that encodes the amino acid sequence of SEQ ID NO: 17, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 17. In some embodiments, the nucleotide sequence encoding the anti-HER2 scFV comprises the sequence set forth in SEQ ID NO: 18, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 18. In some embodiments, the anti-HER2 scFV comprises the amino acid sequence of SEQ ID NO: 17. In some embodiments, the nucleotide sequence encoding the anti-HER2 scFV comprises the nucleotide sequence set forth in SEQ ID NO: 18.

In a specific embodiment, the extracellular antigen binding domain comprises an scFv capable of binding to IL13Rα2. The scFv capable of binding to IL13Rα2 may comprise the amino acid sequence of SEQ ID NO: 29, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 29. In some embodiments, the nucleotide sequence encoding the anti-IL13Rα2 scFV comprises the nucleotide sequence that encodes the amino acid sequence of SEQ ID NO: 29, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 29. In some embodiments, the nucleotide sequence encoding the anti-IL13Rα2 scFV comprises the sequence set forth in SEQ ID NO: 30, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 30. In some embodiments, the anti-IL13Rα2 scFV comprises the amino acid sequence of SEQ ID NO: 29. In some embodiments, the nucleotide sequence encoding the anti-IL13Rα2 scFV comprises the nucleotide sequence set forth in SEQ ID NO: 30.

In a specific embodiment, the extracellular antigen binding domain comprises an scFv capable of binding to EphA2. The scFv capable of binding to EphA2 comprises the amino acid sequence of SEQ ID NO: 38, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 38. In some embodiments, the nucleotide sequence encoding the anti-EphA2 scFV comprises the nucleotide sequence that encodes the amino acid sequence of SEQ ID NO: 38, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 38. In some embodiments, the nucleotide sequence encoding the anti-EphA2 scFV comprises the sequence set forth in SEQ ID NO: 39, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 39. In some embodiments, the anti-EphA2 scFV comprises the amino acid sequence of SEQ ID NO: 38. In some embodiments, the nucleotide sequence encoding the anti-EphA2 scFV comprises the nucleotide sequence set forth in SEQ ID NO: 39.

In some embodiments, the extracellular antigen-binding domain further comprises a leader sequence. The leader sequence may be located at the amino-terminus of the extracellular antigen-binding domain. The leader sequence may be optionally cleaved from the antigen-binding moiety during cellular processing and localization of the CAR to the cellular membrane.

In some embodiments, the leader sequence comprises the amino acid sequence of SEQ ID NO: 15, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 15. In some embodiments, the nucleotide sequence encoding the leader comprises the nucleotide sequence that encodes the amino acid sequence of SEQ ID NO: 15, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 15. In some embodiments, the nucleotide sequence encoding the leader sequence comprises the sequence set forth in SEQ ID NO: 16, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 16. In some embodiments, the leader sequence comprises the amino acid sequence of SEQ ID NO: 15. In some embodiments, the nucleotide sequence encoding the leader sequence comprises the nucleotide sequence set forth in SEQ ID NO: 16. In some embodiments, the nucleotide sequence encoding the leader sequence comprises the sequence set forth in SEQ ID NO: 37, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 37. In some embodiments, the nucleotide sequence encoding the leader sequence comprises the nucleotide sequence set forth in SEQ ID NO: 37.

Transmembrane Domain of the CAR

In some embodiments, the CARs expressed by the modified immune effector cell comprise a transmembrane domain. The transmembrane domain may be fused in frame between the extracellular target-binding domain and the cytoplasmic domain.

The transmembrane domain may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In some embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this invention may be derived from (i.e. comprise at least the transmembrane region(s) of) the α, β or ζ chain of the T-cell receptor, CD28, CD3 epsilon, CD45, CD4, CD5, CD8, CD9, CD16, CD22, CD33, CD37, CD40, CD64, CD80, CD86, CD134, CD137, CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and −β, MB1 (Igα.), B29 or CD3-γ, ζ, or η, in order to retain physical association with other members of the receptor complex.

In some embodiments, the transmembrane domain is derived from CD3ζ, CD28, CD4, or CD8α.

In a specific embodiment, the transmembrane domain is derived from the CD3ζ transmembrane domain. In some embodiments, the CD3ζ transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 23. In some embodiments, the nucleotide sequence that encodes the CD3 transmembrane domain comprises the nucleotide sequence that encodes the amino acid sequence of SEQ ID NO: 23, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 23. In some embodiments, the nucleotide sequence that encodes the CD3 transmembrane domain comprises the nucleotide sequence set forth in SEQ ID NO: 24, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 24. In some embodiments, the CD3ζ transmembrane domain comprises the amino acid sequence set forth in SEQ ID NO: 23. In some embodiments, the nucleotide sequence that encodes the CD3 transmembrane domain comprises the nucleotide sequence set forth in SEQ ID NO: 24.

In a specific embodiment, the transmembrane domain is derived from the CD28 transmembrane domain. In some embodiments, the CD28 transmembrane domain comprises the amino acid sequence set forth in SEQ ID NO: 31, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 31. In some embodiments, the nucleotide sequence that encodes the CD28 transmembrane domain comprises the nucleotide sequence that encodes the amino acid sequence of SEQ ID NO: 31, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 31. In some embodiments, the nucleotide sequence that encodes the CD28 transmembrane domain comprises the nucleotide sequence set forth in SEQ ID: 32, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 32. In some embodiments, the CD28 transmembrane domain comprises the amino acid sequence set forth in SEQ ID NO: 31. In some embodiments, the nucleotide sequence that encodes the CD28 transmembrane domain comprises the nucleotide sequence set forth in SEQ ID NO: 32.

In a specific embodiment, the transmembrane domain is derived from the CD8α transmembrane domain. In some embodiments, the CD8α transmembrane domain comprises the amino acid sequence set forth in SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the CD8α transmembrane domain comprises the nucleotide sequence that encodes the amino acid sequence of SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the CD8α transmembrane domain comprises the nucleotide sequence set forth in SEQ ID NO: 50, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 50. In some embodiments, the CD8α transmembrane domain comprises the amino acid sequence set forth in SEQ ID NO: 49. In some embodiments, the nucleotide sequence that encodes the CD8α transmembrane domain comprises the nucleotide sequence set forth in SEQ ID NO: 50.

In a specific embodiment, the transmembrane domain is derived from the CD4 transmembrane domain. In some embodiments, the CD4 transmembrane domain comprises the amino acid sequence set forth in SEQ ID NO: 51, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 51. In some embodiments, the nucleotide sequence that encodes the CD4 transmembrane domain comprises the nucleotide sequence that encodes the amino acid sequence of SEQ ID NO: 51, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 51. In some embodiments, the nucleotide sequence that encodes the CD4 transmembrane domain comprises the nucleotide sequence set forth in SEQ ID NO: 52, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 52. In some embodiments, the CD4 transmembrane domain comprises the amino acid sequence set forth in SEQ ID NO: 51. In some embodiments, the nucleotide sequence that encodes the CD4 transmembrane domain comprises the nucleotide sequence set forth in SEQ ID NO: 52.

In some embodiments, the CAR further comprises a linker domain between the extracellular antigen-binding domain and the transmembrane domain, wherein the antigen-binding domain, linker, and the transmembrane domain are in frame with each other.

The term “linker domain” as used herein generally means any oligo- or polypeptide that functions to link the antigen-binding moiety to the transmembrane domain. A linker domain can be used to provide more flexibility and accessibility for the antigen-binding moiety. A linker domain may comprise up to 300 amino acids, preferably 10 to 100 amino acids and most preferably 25 to 50 amino acids. A linker domain may be derived from all or part of naturally occurring molecules, such as from all or part of the extracellular region of CD8, CD4 or CD28, or from all or part of an antibody constant region. Alternatively, the linker domain may be a synthetic sequence that corresponds to a naturally occurring linker domain sequence, or may be an entirely synthetic linker domain sequence. Non-limiting examples of linker domains which may be used in accordance to the invention include a part of human CD8α chain, partial extracellular domain of CD28, FcyRllla receptor, IgG, IgM, IgA, IgD, IgE, an Ig hinge, or functional fragment thereof. In some embodiments, additional linking amino acids are added to the linker domain to ensure that the antigen-binding moiety is an optimal distance from the transmembrane domain. In some embodiments, when the linker is derived from an Ig, the linker may be mutated to prevent Fc receptor binding.

In some embodiments, the linker domain comprises a hinge region. In some embodiments, the hinge region comprises the amino acid sequence SEQ ID NO: 19, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 19. In some embodiments, the nucleotide sequence encoding the hinge region comprises the nucleotide sequence that encodes the amino acid sequence of SEQ ID NO: 19, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 19. In some embodiments, the nucleotide sequence encoding the hinge region comprises the sequence set forth in SEQ ID NO: 20, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 20. In some embodiments, the hinge region comprises the amino acid sequence of SEQ ID NO: 19. In some embodiments, the nucleotide sequence encoding the hinge region comprises the nucleotide sequence set forth in SEQ ID NO: 20.

Other hinge regions suitable for use in the present invention may be derived from an immunoglobulin IgG hinge or functional fragment, including IgG1, IgG2, IgG3, IgG4, IgM1, IgM2, IgA1, IgA2, IgD, IgE, or a chimera or variant thereof.

In some embodiments, the linker domain comprises a hinge region which is an IgG1 hinge. In some embodiments, the IgG1 hinge comprises the amino acid sequence SEQ ID NO: 40, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 40. In some embodiments, the nucleotide sequence encoding the IgG1 hinge comprises the nucleotide sequence that encodes the amino acid sequence of SEQ ID NO: 40, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 40. In some embodiments, the nucleotide sequence encoding the IgG1 hinge comprises the sequence set forth in SEQ ID NO: 41, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 41. In some embodiments, the hinge region comprises the amino acid sequence of SEQ ID NO: 40. In some embodiments, the nucleotide sequence encoding the IgG1 hinge comprises the nucleotide sequence set forth in SEQ ID NO: 41.

In some embodiments, the linker domain comprises the amino acid sequence SEQ ID NO: 21, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 21. In some embodiments, the nucleotide sequence encoding the linker domain comprises the nucleotide sequence that encodes the amino acid sequence of SEQ ID NO: 21, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 21. In some embodiments, the nucleotide sequence encoding the linker domain comprises the sequence set forth in SEQ ID NO: 22, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 22. In some embodiments, the linker domain comprises the amino acid sequence of SEQ ID NO: 21. In some embodiments, the nucleotide sequence encoding the linker domain comprises the nucleotide sequence set forth in SEQ ID NO: 22. In some embodiments, the nucleotide sequence encoding the linker domain comprises the sequence set forth in SEQ ID NO: 42, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 42. In some embodiments, the nucleotide sequence encoding the linker domain comprises the nucleotide sequence set forth in SEQ ID NO: 42.

Cytoplasmic Domain of the CAR

In some embodiments, the CAR expressed by the immune effector cell described herein further comprises a cytoplasmic domain. In some embodiments, the cytoplasmic domain of the CAR comprises one or more lymphocyte activation domains.

The cytoplasmic domain, which comprises the lymphocyte activation domain of the CAR, is responsible for activation of at least one of the normal effector functions of the lymphocyte in which the CAR has been placed in. The term “effector function” refers to a specialized function of a cell. Effector function of a T cell, for example, may be cytolytic activity or helper activity including the secretion of cytokines. Thus, the term “lymphocyte activation domain” refers to the portion of a protein which transduces the effector function signal and directs the cell to perform a specialized function. While usually the entire lymphocyte activation domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the lymphocyte activation domain sufficient to transduce the effector function signal.

Non-limiting examples of lymphocyte activation domains which can be used in the CARs described herein include those derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, and MyD88.

In some embodiments, the lymphocyte activation domain is derived from CD3ζ and comprises the amino acid sequence SEQ ID NO: 25. In some embodiments, the CD3t signaling domain comprises the amino acid sequence set forth in SEQ ID NO: 25 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 25. In some embodiments, the nucleotide sequence that encodes the CD3ζ signaling domain comprises the nucleotide sequence that encodes the amino acid sequence of SEQ ID NO: 25, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 25. In some embodiments, the nucleotide sequence that encodes the CD3ζ signaling domain comprises the nucleotide sequence set forth in SEQ ID NO: 26, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 26. In some embodiments, the CD3ζ signaling domain comprises the amino acid sequence set forth in SEQ ID NO: 25. In some embodiments, the nucleotide sequence that encodes the CD3ζ signaling domain comprises the nucleotide sequence set forth in SEQ ID NO: 26. In some embodiments, the nucleotide sequence that encodes the CD3ζ signaling domain comprises the nucleotide sequence set forth in SEQ ID NO: 44, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 44. In some embodiments, the nucleotide sequence that encodes the CD3 signaling domain comprises the nucleotide sequence set forth in SEQ ID NO: 44.

In some embodiments, the cytoplasmic domain further includes one or more co-stimulatory domains. Non-limiting examples of co-stimulatory domains that may be used in the CARs described herein include those derived from 4-1BB (CD137), CD28, ICOS, CD134 (OX-40), BTLA, CD27, CD30, GITR, CD226, and HVEM.

In some embodiments, the co-stimulatory domain is derived from CD28 and comprises the amino acid sequence SEQ ID NO: 33. In some embodiments, the CD28 co-stimulatory domain comprises the amino acid sequence set forth in SEQ ID NO: 33 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 33. In some embodiments, the nucleotide sequence that encodes the CD28 co-stimulatory domain comprises the nucleotide sequence that encodes the amino acid sequence of SEQ ID NO: 33, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 33. In some embodiments, the nucleotide sequence that encodes the CD28 co-stimulatory domain comprises the nucleotide sequence set forth in SEQ ID NO: 34, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 34. In some embodiments, the CD28 co-stimulatory domain comprises the amino acid sequence set forth in SEQ ID NO: 34. In some embodiments, the nucleotide sequence that encodes the CD28 co-stimulatory domain comprises the nucleotide sequence set forth in SEQ ID NO: 34.

In some embodiments, the cytoplasmic domain comprises both the CD3ζ lymphocyte activation domain and the CD28 co-stimulatory domain, which are fused in frame. The CD3ζ lymphocyte activation domain and the CD28 co-stimulatory domain can be in any order. In some embodiments, the CD3ζ lymphocyte activation domain is downstream of the CD28 co-stimulatory domain.

Accessory Genes of the CAR

In addition to the CAR construct, the CAR may further comprise an accessory gene that encodes an accessory peptide. Examples of accessory genes can include a transduced host cell selection marker, an in vivo tracking marker, a cytokine, a suicide gene, or some other functional gene. In a specific embodiment, the CAR is co-expressed with a truncated CD19 molecule (tCD19). For example, expression of tCD19 can help determine transduction efficiency. In some embodiments, the CAR comprises the tCD19 construct. In some embodiments, the CAR does not include the tCD19 construct. In some embodiments, the tCD19 can be replaced with a functional accessory gene to enhance the effector function of the CAR containing immune effector cells. In some embodiments, the functional accessory gene can increase the safety of the CAR. In some embodiments, the CAR comprises at least one accessory gene. In some embodiments, the CAR comprises one accessory gene. In other embodiments, the CAR comprises two accessory genes. In yet another embodiment, the CAR comprises three accessory genes.

Non-limiting examples of classes of accessory genes that can be used to increase the effector function of CAR containing immune effector cells, include i) secretable cytokines (e.g., but not limited to, IL-7, IL-12, IL-15, IL-18), ii) membrane bound cytokines (e.g., but not limited to, IL-15), iii) chimeric cytokine receptors (e.g., but not limited to, IL-2/IL-7, IL-4/IL-7), iv) constitutive active cytokine receptors (e.g., but not limited to, C7R), v) dominant negative receptors (DNR; e.g., but not limited to TGFRII DNR), vi) ligands of costimulatory molecules (e.g., but not limited to, CD80, 4-1BBL), vii) antibodies, including fragments thereof and bispecific antibodies (e.g., but not limited to, bispecific T-cell engagers (BiTEs)), or vii) a second CAR.

In some embodiments, the accessory gene included herein is a truncated CD19 molecule (tCD19). In some embodiments, the tCD19 molecule comprises the amino acid sequence set forth in SEQ ID NO: 49 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the tCD19 molecule comprises the nucleotide sequence that encodes the amino acid sequence of SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: v. In some embodiments, the nucleotide sequence that encodes the tCD19 molecule comprises the nucleotide sequence set forth in SEQ ID NO: 50, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 50. In some embodiments, the tCD19 molecule comprises the amino acid sequence set forth in SEQ ID NO: 49. In some embodiments, the nucleotide sequence that encodes the tCD19 molecule comprises the nucleotide sequence set forth in SEQ ID NO: 50.

tCD19 may be separated from the CAR-encoding sequence by a separation sequence (e.g., a 2A sequence). tCD19 could also be replaced with two accessory genes separated by a separation sequence (e.g., a 2A sequence) using a combination of the classes of molecules listed above (e.g., CAR-2A-CD20-2A-IL15). In addition, the use of two separation sequences (e.g., 2A sequences) would allow the expression of TCR (e.g., CAR-2A-TCRα-2A-TCRβ). In the constructs with a CAR and two or three accessory genes, the order of the CAR and the 2nd or 3rd transgene could be switched.

A “separation sequence” refers to a peptide sequence that causes a ribosome to release the growing polypeptide chain that it is being synthesizes without dissociation from the mRNA. In this respect, the ribosome continues translating and therefore produces a second polypeptide. Non-limiting examples of separation sequences includes T2A (EGRGSLLTCGDVEENPGP (SEQ ID NO: 45) or GSGEGRGSLLTCGDVEENPGP (SEQ ID NO: 53)); the foot and mouth disease virus (FMDV) 2A sequence (GSGSRVTELLYRMKRAETYCPRPLLAIHPTEARHKQKIVAPVKQLLNFDLLKLAGD VESNPGP (SEQ ID NO: 54)); Sponge (Amphimedon queenslandica) 2A sequence (LLCFLLLLLSGDVELNPGP (SEQ ID NO: 55); or HHFMFLLLLLAGDIELNPGP (SEQ ID NO: 56)); acorn worm (Saccoglossus kowalevskii) 2A sequence (WFLVLLSFILSGDIEVNPGP (SEQ ID NO: 57)); amphioxus (Branchiostoma floridae) 2A sequence (KNCAMYMLLLSGDVETNPGP (SEQ ID NO: 58); or MVISQLMLKLAGDVEENPGP (SEQ ID NO: 59)); porcine teschovirus-1 2A sequence (GSGATNFSLLKQAGDVEENPGP (SEQ ID NO: 60)); and equine rhinitis A virus 2A sequence (GSGQCTNYALLKLAGDVESNPGP (SEQ ID NO: 61)). In some embodiments, the separation sequence is a naturally occurring or synthetic sequence. In some embodiments, the separation sequence includes the 2A consensus sequence D-X-E-X-NPGP (SEQ ID NO: 62), in which X is any amino acid residue.

Non-Limiting Examples of CARs

In some embodiments, the CAR can be encoded by one polynucleotide chain. In some embodiments, the CAR of the invention is encoded by a nucleotide sequence comprising the nucleotides sequence of SEQ ID NO: 4, 6, 10, 12, or 14, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 4, 6, 10, 12, or 14. In some embodiments, the CAR comprises the amino acid sequence of SEQ ID NO: 3, 5, 9, 11, or 13, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 3, 5, 9, 11, or 13.

### Methods for Generating Modified Immune Effector Cells

In one aspect, the present invention provides a method for generating a modified immune effector cell described herein. Such method includes deleting or modifying a DNMT3A gene or gene product in the cell so that the DNMT3A-mediated de novo DNA methylation of the cell genome is inhibited.

In some embodiments, the DNMT3A gene in the immune effector cell is deleted or modified as a result of an activity of a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease.

Site-specific nucleases may create double-strand breaks or single-strand breaks (i.e., nick) in a genomic DNA of a cell. Although not wishing to be bound by theory, these breaks are typically repaired by the cell using one of two mechanisms: non-homologous end joining (NHEJ) and homology-directed repair (HDR). In NHEJ, the double-strand breaks are repaired by direct ligation of the break ends to one another. As a result, no new nucleic acid material is inserted into the site, although a few bases may be lost or added, resulting in a small insertions and deletion (indel). In HDR, a donor polynucleotide with homology to the cleaved target DNA sequence is used as a template to repair the cleaved target DNA sequence, resulting in the transfer of genetic information from the donor polynucleotide to the target DNA. As such, new nucleic acid material may be inserted or copied into the cleavage site. In some cases, an exogenous donor polynucleotide can be provided to the cell. The modifications of the target DNA due to NHEJ and/or HDR may lead to, for example, gene correction, gene replacement, gene tagging, transgene insertion, nucleotide deletion, gene disruption, gene mutation, sequence replacement, etc. Accordingly, cleavage of DNA by a site-directed nuclease may be used to delete nucleic acid material from a target DNA sequence by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material (e.g., a transgene) into a locus of choice in the target DNA.

In some embodiments, the site-specific nuclease is an RNA-guided endonuclease. In particular, a group of RNA-guided endonucleases known as CRISPR-associated (Cas) proteins may be employed to genetically modify the immune effector cell. A Cas protein may form an RNA-protein complex (referred to as RNP) with a guide RNA (gRNA) and is capable of cleaving a target site bearing sequence complementarity to a short sequence (typically about 20-40 nt) in the gRNA. In some embodiments, the RNA-guided endonuclease is a Cas9 protein, Cpf1 (Cas12a) protein, C2c1 protein, C2c3 protein, or C2c2 protein.

In a specific embodiment, the RNA-guided endonuclease is a Cas9 protein. The Cas9 protein may be from S. pyogenes, Streptococcus thermophilus, Neisseria meningitidis, F. novicida, S. mutans or Treponema denticola. The Cas9 may be a native or modified Cas9 protein.

The Cas9 protein may be programmed with a gRNA that targets a locus with or near the DNMT3A gene. In some embodiments, the gRNA comprises a nucleotide sequence encoded by SEQ ID NO: 63, or SEQ ID NO: 68.

In alternative embodiments, the site-specific nuclease used in the methods described herein is a zinc finger nuclease, a TALEN nuclease, or a mega-TALEN nuclease.

In some embodiments, the DNMT3A gene product in the immune effector cell is deleted or modified as a result of an activity of an RNA interference (RNAi) molecule or an antisense oligonucleotide. RNA interference (RNAi) refers to the process of sequence-specific post-transcriptional gene silencing in animals mediated by small interfering RNAs (siRNAs) (Fire et al., 1998, Nature, 391, 806; Hamilton et al., 1999, Science, 286, 950-951). Any small nucleic acid molecules capable of mediating RNAi, such as a short interfering nucleic acid (siNA), a small interfering RNA (siRNA), a double-stranded RNA (dsRNA), a micro-RNA (miRNA), and a short hairpin RNA (shRNA), may be to inhibit the expression of the DNMT3A gene. An antisense oligonucleotide (ASO) is a short nucleotide sequence that can hybridize or bind (e.g., by Watson-Crick base pairing) in a complementary fashion to its target sequence.

In some embodiments, the RNAi molecule is a small interfering RNA (siRNA) or a small hairpin RNA (shRNA). siRNAs, also known as short interfering RNA or silencing RNA, are a class of double-stranded RNA molecules, 20-25 base pairs in length, and operating within the RNA interference (RNAi) pathway. shRNAs or short hairpin RNAs are a group of artificial RNA molecules with a tight hairpin turn that can be used to silence target gene expression via RNA interference (RNAi).

In various embodiments, the site-specific nuclease, the RNAi molecule or the antisense oligonucleotide as described above is introduced into the immune effector cell via a viral vector, a non-viral vector or a physical means.

The methods for generating a modified immune effector cell described herein may further includes activating the STAT5 signaling pathway in the immune effector cell by a signaling molecule. In some embodiments, the signaling molecule is a common gamma chain cytokine. Non-limiting examples of cytokines that may be used in the methods described herein include IL-15, IL-7, IL-2, IL-4, IL-9, and IL-21.

In some embodiments, the STAT5 signaling pathway is activated by modifying the immune effector cell to express a constitutively active cytokine receptor or a switch receptor. Such constitutively active cytokine receptor may be a constitutively active IL7 receptor (C7R). Such switch receptor may be an IL-4/IL-7 receptor or an IL-4/IL-2 receptor.

In some embodiments, the immune effector cell is contacted with an effective amount of the signaling molecule or a carrier containing the signaling molecule. Suitable carriers include, but are not limited to, polymers, micelles, reverse micelles, liposomes, emulsions, hydrogels, microparticles, nanoparticles, and microspheres. In some embodiments, the carrier is a nanoparticle.

In some embodiments, the immune effector cell is contacted with the signaling molecule more than once. The immune effector cell may be contacted with the signaling molecule 2 times, 3 times, 4 times, 5 times, 6 times, 7 times, 8 times, or more than 8 times. The immune effector cell may be contacted with the signaling molecule at a frequency of every 8 hours, every 12 hours, every 16 hours, every 24 hours, every 2 days, every 3 days, every 4 days, every 5 days, every 6 days, every 7 days, every 8 days, every 8 days, every 10 days, once a week, twice a week, biweekly, once a month, twice a month, 3 times a month, 4 times a month, or 5 times a month.

In some embodiments, the signaling molecule is expressed in the immune effector cell. The signaling molecule may be expressed from a transgene introduced into the immune effector cell. The signaling molecule-expressing transgene may be introduced into the immune effector cell using a viral vector, a non-viral vector or a physical means.

In some embodiments, the modified immune effector cell is further engineered to express a chimeric antigen receptor (CAR) as described herein. The CAR may comprise an extracellular antigen-binding domain, a transmembrane domain, and/or a cytoplasmic domain as described above. The CAR may be expressed from a transgene introduced into the immune effector cell. The CAR-expressing transgene may be introduced into the immune effector cell using a viral vector, a non-viral vector or a physical means.

In some embodiments, the immune effector cells are T cells.

In some embodiments, the immune effector cells are NK cells.

In some embodiments, the immune effector cells are stem cells that are capable of differentiating into immune cells, including induced pluripotent stem cells (iPSCs).

Modified immune effector cells can be activated and/or expanded ex vivo for use in adoptive cellular immunotherapy in which infusions of such cells have been shown to have anti-disease reactivity in a disease-bearing subject. The compositions and methods of this invention can be used to generate a population of immune effector cells (e.g., T lymphocyte or natural killer cells) with enhanced immune cell function for use in immunotherapy in the treatment of the disease.

Isolation/Enrichment

The immune effector cells may be autologous/autogeneic (“self”) or non-autologous (“non-self,” e.g., allogeneic, syngeneic or xenogeneic). In some embodiments, the immune effector cells are obtained from a mammalian subject. In other embodiments, the immune effector cells are obtained from a primate subject. In some embodiments, the immune effector cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In some embodiments, lymphocytes can be obtained from blood collected from a subject using techniques generally known to the skilled person, such as sedimentation, e.g., FICOLL™ separation.

In some embodiments, cells from the circulating blood of a subject are obtained by apheresis. An apheresis device typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In some embodiments, immune effector cells can be isolated from peripheral blood mononuclear cells (PBMCs) by lysing the red blood cells and depleting the monocytes. As an example, the cells can be sorted by centrifugation through a PERCOLL™ gradient. In some embodiments, after isolation of PBMC, both cytotoxic and helper T lymphocytes can be sorted into naive, memory, and effector T cell subpopulations either before or after activation, expansion, and/or genetic modification.

In some embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In some embodiments, the T lymphocytes for use in the compositions of the invention do not express or do not substantially express one or more of the following markers: CD57, CD244, CD160, PD-1, CTLA4, TIM3, and LAG3.

In some embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

Stimulation/Activation

In order to reach sufficient therapeutic doses of immune effector cell compositions, immune effector cells are often subjected to one or more rounds of stimulation/activation. In some embodiments, a method of producing immune effector cells for administration to a subject comprises stimulating the immune effector cells to become activated in the presence of one or more stimulatory signals or agents (e.g., compound, small molecule, e.g., small organic molecule, nucleic acid, polypeptide, or a fragment, isoform, variant, analog, or derivative thereof). In some embodiments, a method of producing immune effector cells for administration to a subject comprises stimulating the immune effector cells to become activated and to proliferate in the presence of one or more stimulatory signals or agents.

Immune effector cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the T cell based immune effector cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100, which is incorporated herein by reference in its entirety). Other antibodies which bind to the same epitopes as any of the above described antibodies can also be used.

In some embodiments, the immune effector cells are activated by administering phorbol myristate acetate (PMA) and ionomycine. In some embodiments, the immune effector cells are activated by administering an appropriate antigen that induces activation and then expansion. In some embodiments, PMA, ionomycin, and/or appropriate antigen are administered with CD3 induce activation and/or expansion.

In general, the activating agents used in the present invention includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441, which is incorporated herein by reference in its entirety), decabodies (Stone, E., et al., Journal of Immunological Methods (2007) 318, 88-94, which is incorporated herein by reference in its entirety) and other domain antibodies (Holt, L. J., et al., Trends Biotechnol. (2003), 21, 11, 484-490, which is incorporated herein by reference in its entirety). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In some embodiments, one or more binding sites of the CD3ζ agents may be a bivalent proteinaceous artificial binding molecule such as a dimeric lipocalin mutein (i.e., duocalin). In some embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3− antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In some embodiments, the activating agent (e.g., CD3-binding agents) can be present in a concentration of about 0.1 to about 10 μg/ml. In some embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In some embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In some embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, and 8,877,182; U.S. Patent Applications US2004/0058445, US2007/0160578, US2013/0011376, US2015/0118207, and US2015/0037887; and PCT Patent Application WO2016/122147, each of which is incorporated herein by reference in its entirety.

In some embodiments, the NK based immune effector cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In some embodiments, the NK based immune effector cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In some embodiments, the NK activating agent can be present in a concentration of about 0.1 to about 10 μg/ml. In some embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In some embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In some embodiments, the NK activating agent can be present in a concentration of 1 μg/ml.

In some embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

Polynucleotide and/or Polypeptide Transfer

In some embodiments, the immune effector cells are genetically modified to by introducing polynucleotides and/or polypeptide (e.g., a CAR, a signaling molecule, site-specific nuclease, an RNAi molecule or an antisense oligonucleotide, or polynucleotides encoding the same). The immune effector cells can be genetically modified after stimulation/activation. In some embodiments, the immune effector cells are modified within 12 hours, 16 hours, 24 hours, 36 hours, or 48 hours of stimulation/activation. In some embodiments, the cells are modified within 16 to 24 hours after stimulation/activation. In some embodiments, the immune effector cells are modified within 24 hours.

In order to genetically modify the immune effector cell, the polynucleotides and/or polypeptide (e.g., a CAR, a signaling molecule, site-specific nuclease, an RNAi molecule or an antisense oligonucleotide, or polynucleotides encoding the same) must be transferred into the host cell. Polynucleotide and/or polypeptide transfer may be via viral, non-viral gene delivery methods, or a physical method. Suitable methods for polynucleotide and/or polypeptide delivery for use with the current methods include any method known by those of skill in the art, by which a polynucleotide and/or polypeptide can be introduced into an organelle, cell, tissue or organism.

In various embodiments, polypeptides or polynucleotides (e.g., a CAR, a signaling molecule, site-specific nuclease, an RNAi molecule or an antisense oligonucleotide, or polynucleotides encoding the same) described in the present invention are introduced to the immune effector cell via a recombinant vector.

In some embodiments, the recombinant vector encoding a CAR described above comprises the nucleotide sequence of SEQ ID NO: 4, 6, 10, 12, or 14, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 4, 6, 10, 12, or 14. In some embodiments, the recombinant vector comprises the nucleotide sequence that encodes the amino acid sequence of SEQ ID NO: 3, 5, 9, 11, or 13, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% sequence identity with SEQ ID NO: 3, 5, 9, 11, or 13.

In some embodiments, the vector is a viral vector. Suitable viral vectors that can be used in the present invention include, but are not limited to, a retroviral vector, an adenoviral vector, an adeno-associated viral (AAV) vector, a herpes viral vector, or a baculoviral vector. In one specific embodiment, the viral vector is a lentiviral vector.

In some embodiments, the immune effector cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity. In some embodiments, the immune effector cells may be removed from the subject and transfected ex vivo using the polynucleotides (e.g., expression vectors) of the invention. In some embodiments, the immune effector cells obtained from the subject can be transfected or transduced with the polynucleotides (e.g., expression vectors) of the invention and then administered back to the subject.

In some embodiments, polynucleotides and/or polypeptides are transferred to the cell in a non-viral vector. In some embodiments, the non-viral vector is a transposon. Exemplary transposons hat can be used in the present invention include, but are not limited to, a sleeping beauty transposon and a PiggyBac transposon.

Nucleic acid vaccines may also be used to transfer polynucleotides into the immune effector cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In some embodiments, the polynucleotide(s) is operatively linked to at least one regulatory element for expression of the gene product (e.g., a CAR, a signaling molecule, site-specific nuclease, an RNAi molecule). The regulatory element can be capable of mediating expression of the gene product in the host cell (e.g., modified immune effector cell). Regulatory elements include, but are not limited to, promoters, enhancers, initiation sites, polyadenylation (polyA) tails, IRES elements, response elements, and termination signals. In some embodiments, the regulatory element regulates expression of the gene product. In some embodiments, the regulatory element increased the expression of the gene product. In some embodiments, the regulatory element increased the expression of the gene product once the host cell (e.g., modified immune effector cell) is activated. In some embodiments, the regulatory element decreases expression of the gene product. In some embodiments, the regulatory element decreases expression of the gene product once the host cell (e.g., modified immune effector cell) is activated.

In various embodiment, polypeptides or polynucleotides (e.g., a CAR, a signaling molecule, site-specific nuclease, an RNAi molecule or an antisense oligonucleotide, or polynucleotides encoding the same) are introduced into the modified immune effector cell using a physical means. Suitable physical means include, but are not limited to, electroporation, microinjection, magnetofection, ultrasound, a ballistic or hydrodynamic method, or a combination thereof.

Electroporation is a method for polynucleotide and/or polypeptide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In some embodiments, cell wall-degrading enzymes, such as pectin-degrading enzymes, can be employed to render the immune effector cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Another method for polynucleotide and/or polypeptide transfer includes injection. In some embodiments, a polypeptide, a polynucleotide or viral vector may be delivered to a cell, tissue, or organism via one or more injections (e.g., a needle injection). Non-limiting methods of injection include injection of a composition (e.g., a saline based composition). Polynucleotides and/or polynucleotides can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Additional methods of polynucleotide and/or polypeptide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide and/or polypeptide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and/or polypeptide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

Expansion/Proliferation

After the immune effector cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120, each of which is incorporated by reference in their entirety for all purposes). Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T cells are CD62L+CD8+ specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the agent(s) used for expansion (e.g., IL-7, IL-15) are administered at about 20 units/ml to about 200 units/ml. In some embodiments, the agent(s) used for expansion (e.g., IL-7, IL-15) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In some embodiments, the agent(s) used for expansion (e.g., IL-7, IL-15) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In some embodiments, the agent(s) used for expansion (e.g., IL-7, IL-15) are administered at about 5 mg/ml to about 10 ng/ml. In some embodiments, the agent(s) used for expansion (e.g., IL-7, IL-15) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In some embodiments, the agent(s) used for expansion (e.g., IL-7, IL-15) are administered at about 5 ng/ml, 6 ng/ml, 7 ng/ml, 8 ng/ml, 9, ng/ml, or 10 ng/ml.

After the immune effector cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In some embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is incorporated by reference in their entirety for all purposes).

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for immune effector cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

### Methods of Enhancing Immune Cell Function

In one aspect, the present invention provides a method of enhancing an antitumor activity of an immune effector cell, including the steps of a) modifying a DNMT3A gene or gene product in the cell so that the DNMT3A-mediated de novo DNA methylation of the cell genome is inhibited; and b) activating the STAT5 signaling pathway in the cell by either stimulating the cell with a signaling molecule or genetically modifying the cell to express a signaling molecule. In some embodiments, the immune effector cell is a T cell. In some embodiments, the immune effector cell is a NK cell.

In some embodiments, the DNMT3A gene in the immune effector cell is deleted or modified as a result of an activity of a site-specific nuclease. In some embodiments, the site-specific nuclease is an RNA-guided endonuclease. In some embodiments, the RNA-guided endonuclease is a Cas9 protein. In one embodiment, the Cas9 protein is programmed with a gRNA that comprises a nucleotide sequence encoded by SEQ ID NO: 63 or SEQ ID NO: 68. In alternative embodiments, the site-specific nuclease used in the methods described herein is a zinc finger nuclease, a TALEN nuclease, or a mega-TALEN nuclease.

In some embodiments, the DNMT3A gene product in the immune effector cell is deleted or modified as a result of an activity of an RNA interference (RNAi) molecule or an antisense oligonucleotide. In some embodiments, the RNAi molecule is a small interfering RNA (siRNA) or a small hairpin RNA (shRNA).

In various embodiments, the site-specific nuclease, the RNAi molecule or the antisense oligonucleotide as described above is introduced into the immune effector cell via a viral vector, a non-viral vector or a physical means described herein.

In some embodiments, the signaling molecule for activating the STAT5 signaling pathway in the cell is a common gamma chain cytokine. In some embodiments, the cytokine is IL-15, IL-7, IL-2, IL-4, IL-9, or IL-21.

In some embodiments, the immune effector cell is contacted with an effective amount of the signaling molecule or a carrier containing the signaling molecule. Suitable carriers include, but are not limited to, polymers, micelles, reverse micelles, liposomes, emulsions, hydrogels, microparticles, nanoparticles, and microspheres. In some embodiments, the carrier is a nanoparticle.

In some embodiments, the immune effector cell is contacted with the signaling molecule more than once. The immune effector cell may be contacted with the signaling molecule 2 times, 3 times, 4 times, 5 times, 6 times, 7 times, 8 times, or more than 8 times. The immune effector cell may be contacted with the signaling molecule at a frequency of every 8 hours, every 12 hours, every 16 hours, every 24 hours, every 2 days, every 3 days, every 4 days, every 5 days, every 6 days, every 7 days, every 8 days, every 8 days, every 10 days, once a week, twice a week, biweekly, once a month, twice a month, 3 times a month, 4 times a month, or 5 times a month.

In some embodiments, the signaling molecule is expressed in the immune effector cell. The signaling molecule may be expressed from a transgene introduced into the immune effector cell. The signaling molecule-expressing transgene may be introduced into the immune effector cell using a viral vector, a non-viral vector or a physical means described herein.

In further embodiments, the STAT5 signaling pathway is activated by modifying the immune effector cell to express a constitutively active cytokine receptor or a switch receptor. Such constitutively active cytokine receptor may be a constitutively active IL7 receptor (C7R). Such switch receptor may be an IL-4/IL-7 receptor or an IL-4/IL-2 receptor.

In some embodiments, the method further includes genetically modifying the immune effector cell to express a chimeric antigen receptor (CAR) that is capable of binding to an antigen specific for the tumor. Non-limiting examples of the CARs include any of those described herein.

In some embodiments, the method further includes activation and/or expansion of the immune effector cell ex vivo.

### Pharmaceutical Compositions

In some embodiments, the compositions comprise one or more polypeptides, polynucleotides, vectors comprising same, and cell compositions, as disclosed herein. Compositions include, but are not limited to pharmaceutical compositions. In some embodiments, the compositions of the present invention comprise an amount of modified immune effector cells manufactured by the methods disclosed herein.

In one aspect, the present invention provides a pharmaceutical composition comprising a modified immune effector cell described herein and a pharmaceutically acceptable carrier and/or excipient. Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified immune effector cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified immune effector cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified immune effector cells may be mixed with substances that adhere or penetrate then prior to their administration, e.g., but not limited to, nanoparticles.

### Therapeutic Methods

In one aspect, the present invention provides a method of treating a disease or disorder in a subject in need thereof, including administering to the subject an effective amount of the modified immune effector cells or the pharmaceutical composition described herein. In some embodiments, the modified immune effector cells are prepared by the methods as disclosed above.

In some embodiments, the modified immune effector cell is an autologous cell. In some embodiments, the modified immune effector cell is an allogeneic cell.

In some embodiments, the disease being treated by the therapeutic methods described herein is a cancer, an infectious disease, an inflammatory disorder, or an autoimmune disease.

The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of cancer can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the cancer is a solid tumor. In some embodiments, the cancer is a breast, prostate, urinary bladder, skin, lung, ovary, sarcoma, or brain cancer.

The therapeutic methods described herein may be used to treat a cancer expressing HER2, IL13Rα2, or EphA2. Cancers expressing HER2 may include, but are not limited to, sarcomas such as angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, or synovial sarcoma; brain cancers such as glioblastoma; breast, prostate, lung, and colon cancers or epithelial cancers/carcinomas such as breast cancer, colon cancer, prostate cancer, head and neck cancer, skin cancer; cancers of the genitourinary tract such as ovarian cancer, endometrial cancer, cervical cancer and kidney cancer; lung cancer, gastric cancer, cancer of the small intestine, liver cancer, pancreatic cancer, gall bladder cancer, cancers of the bile duct, esophagus cancer, cancer of the salivary glands and cancer of the thyroid gland. In some embodiments, the cancer is a HER2-positive breast cancer.

Cancers expressing IL13Rα2 may include, but are not limited to, brain cancers such as glioblastoma, colon cancer, renal cell carcinoma, pancreatic cancer, melanoma, head and neck cancer, mesothelioma, and ovarian cancer. In some embodiments, the cancer is an IL13Rα2-positive glioblastoma.

Cancers expressing EphA2 may include, but are not limited to, sarcomas such as rhabdomyosarcoma, osteosarcoma, and Ewings sarcoma; breast, prostate, urinary bladder, skin cancers including melanoma, lung cancer, liver cancer, ovarian cancer, stomach cancer, colorectal cancer, thyroid cancer, head and neck cancer, cervical cancer, pancreatic cancer, endometrial cancer, and brain cancers.

The therapeutic methods described herein may include the steps of a) isolating an immune effector cell from the subject or a donor; b) modifying a DNMT3A gene or gene product in the immune effector cell such that the DNMT3A-mediated de novo DNA methylation of the cell genome is inhibited; c) activating the STAT5 signaling pathway in the immune effector cell by either stimulating the immune effector cell with a signaling molecule or genetically modifying the immune effector cell to express a signaling molecule; and d) introducing the modified immune effector cell into the subject before or after step (c).

Activating the STAT5 signaling pathway in the immune effector cell may be achieved by stimulating the immune effector cell with a signaling molecule either ex vivo or in vivo. For example, stimulating the immune effector cell with a signaling molecule may be carried out by mixing the immune effector cell directly with the signaling molecule, or with a carrier (e.g., nanoparticles) containing the signaling molecule ex vivo. Mixing of the immune effector cell with the signaling molecule, or with a carrier (e.g., nanoparticles) containing the signaling molecule may be carried out prior to administration, or during administration. In some embodiments, the immune effector cells may be administered with nanoparticle “backpacks” which are capable of carrying signaling molecules and attaching them to the immune effector cells. Such nanoparticle “backpacks” may selectively release the signaling molecules in response to certain stimuli, such as the activation of the immune effector cell (Tang L., Nat Biotechnol. 2018; 36(8):707-716, which is incorporated by reference in their entirety for all purposes).

Alternatively, signaling molecules may be provided to the modified immune effector cells in vivo by administration of the signaling molecule, for example systemically, to the subject such that the signaling molecule can ultimately contact the modified immune effector cells. Signaling molecules may also be provided to the modified immune effector cells in vivo using oncolytic viruses encoding the signaling molecule. Oncolytic viruses can selectively infect and/or lyse cancer or tumor cells as compared to normal cells. Exemplary oncolytic viruses include herpes simplex virus-1, herpes simplex virus-2, a vesicular stomatitis virus, and a vaccinia virus.

Activating the STAT5 signaling pathway in the immune effector cell may also be achieved by genetically modifying the immune effector cell to express a signaling molecule. The signaling molecule may be expressed from a transgene introduced into the immune effector cell. Alternatively, the STAT5 signaling pathway is activated by modifying the immune effector cell to express a constitutively active cytokine receptor or a switch receptor. Such constitutively active cytokine receptor may be a constitutively active IL7 receptor (C7R). Such switch receptor may be an IL-4/IL-7 receptor or an IL-4/IL-2 receptor.

In some embodiments, the therapeutic methods include genetically modifying the immune effector cell to express a chimeric antigen receptor (CAR) that is capable of binding specifically to an antigen.

In cases where the immune effector cell is isolated from a donor, the method may further include a method to prevent graft vs host disease (GVHD) and the immune effector cell rejection.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of immune effector cells. It is also contemplated that a variety of doses will be effective to improve in vivo effector function of immune effector cells.

In some embodiments, composition comprising the immune effector cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of immune effector cells will depend on the therapeutic use for which the composition is intended for.

Modified immune effector cells may be administered multiple times at dosages listed above. The immune effector cells may be allogeneic, syngeneic, xenogeneic, or autologous to the patient undergoing therapy.

The compositions and methods described in the present disclosure may be utilized in conjunction with other types of therapy for cancer, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the invention can be combined with other therapies that block inflammation (e.g., via blockage of ILL INFα/β, IL6, TNF, IL23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD dimers or larger polymers of CD either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating cancer, the compositions of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the immune effector cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present invention include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

Below are the materials and methods used in Examples 1-15.

### Cell Lines

U373 glioma and 293T (human embryonic kidney) cells were purchased from the American Type Culture Collection (ATCC) (Manassas, Va.). LM7 osteosarcoma cells were kindly provided by Dr. Eugenie Kleinerman (MD Anderson Cancer Center, Houston, Tex.) in 2011. U373 cells were maintained in RPMI 1640 supplemented with 10% fetal bovine serum and 2 mmol/L GlutaMAX while LM7 and 293T were maintained in Dulbecco's Modified Eagle Medium (DMEM) with the same supplements. The production of U373 and LM7 cells expressing enhanced green fluorescent protein and firefly luciferase (eGFP.ffLuc) has been previously described (35, 56). Cell lines were routinely validated using the ATCC STR Profiling Cell Authentication Service and tested for mycoplasma on a regular basis.

### Production of Retroviral Vectors

The generation of retroviral vectors encoding HER2-, IL13Rα2-, or EphA2-CARs with a CD28 transmembrane domain and CD28.ζ signaling domain, or IL-15 was previously described (35, 57-59). The retroviral vector encoding the first generation HER2.ζ-CAR was created by subcloning the HER2-specific scFv, FRP5 (60, 61), into a retroviral vector encoding an expression cassette with a CD28 transmembrane and ζ signaling domain (62). RD114-pseudotyped retroviral particles were generated as previously described (56) by transient transfection of 293T cells using GeneJuice Transfection Reagent (EMD Millipore, Burlington, Mass.). Viral supernatants were collected 48 hours post-transfection for the same day T cell transduction or snap-frozen, and stored at −80° C. until use.

### Generation of DNMT3A Knockout CAR T Cells

This study was conducted with approval from the St. Jude Children's Research Hospital Institutional Review Board (IRB). Peripheral blood mononuclear cells (PBMC) were isolated from consented healthy donors (IRB XPD15-086) via density gradient separation using Lymphoprep (StemCell Technologies, Vancouver, BC). Cells were then plated in 24 well non tissue culture-treated plates pre-coated with 250 ng each of anti-CD3 and anti-CD28 monoclonal antibodies (Miltenyi Biotec, Bergisch Gladbach, Germany). Culture medium for initial stimulation was RPMI 1640 supplemented with 10% fetal bovine serum and 2 mmol/L GlutaMAX (Thermo Fisher, Waltham, Mass.). IL-7 and IL-15 were added at 10 ng/mL and 5 ng/mL, respectively, 24 hours later. The following day, cells were transduced on RetroNectin (Takara Bio, Mountain View, Calif.)-coated plates and after 24 hours electroporated with S. pyogenes Cas9-single guide RNA RNP complexes targeting DNMT3A or mCherry (Control). Guide RNAs were purchased from Synthego (Menio Park, Calif.) and recombinant Cas9 was purchased from the Macro Lab at the University of California, Berkeley. Electroporation was performed using the Neon Transfection System (1600V, 3 pulses, 10 ms) according to the manufacturer's protocol (Thermo Fisher, Waltham, Mass.). Electroporated T cells were left to recover in RPMI 1640 supplemented with 20% FBS, Glutamax, 10 ng/mL IL-7, and 5 ng/mL IL-15 for 72 hours. Following recovery, the media was switched to RPMI 1640 containing 10% FBS and GlutaMAX. The cells were then expanded for 10-12 days with IL-7 and IL-15 added every 2-3 days at the same concentrations indicated above.

### Guide RNA Design and Validation

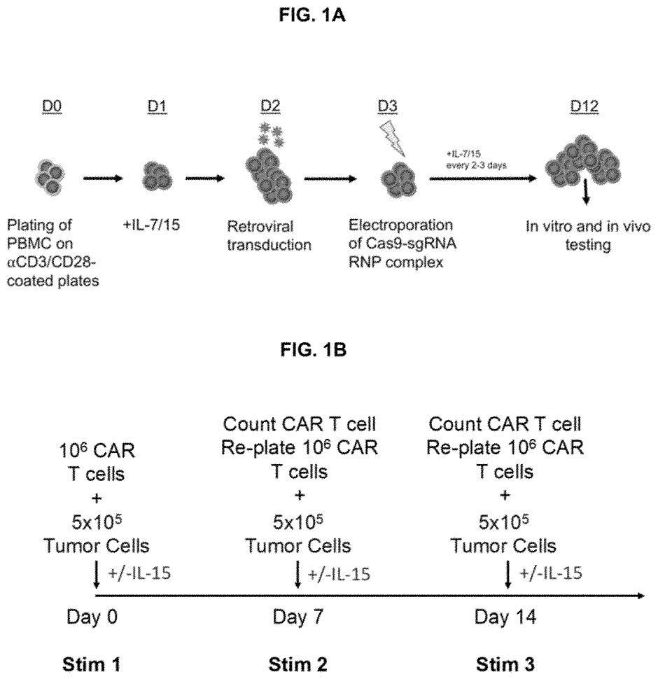

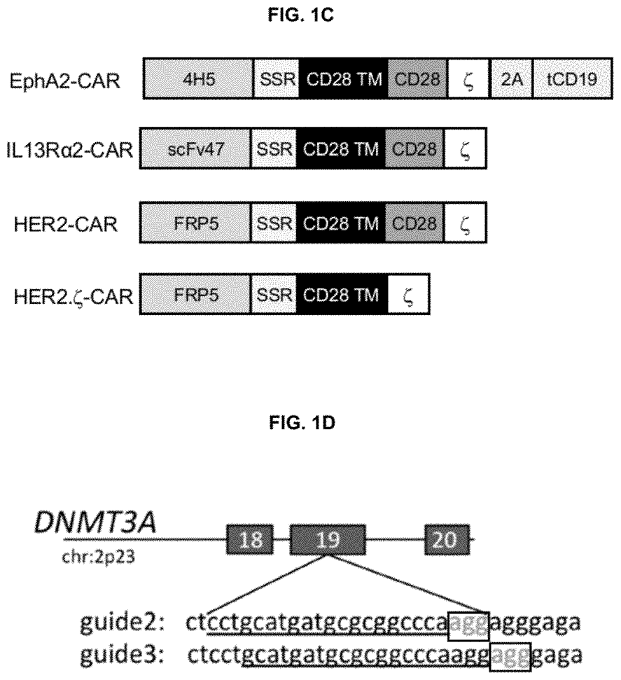

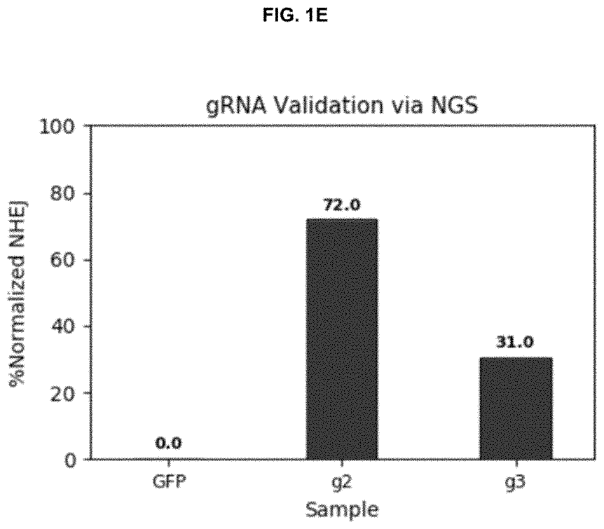

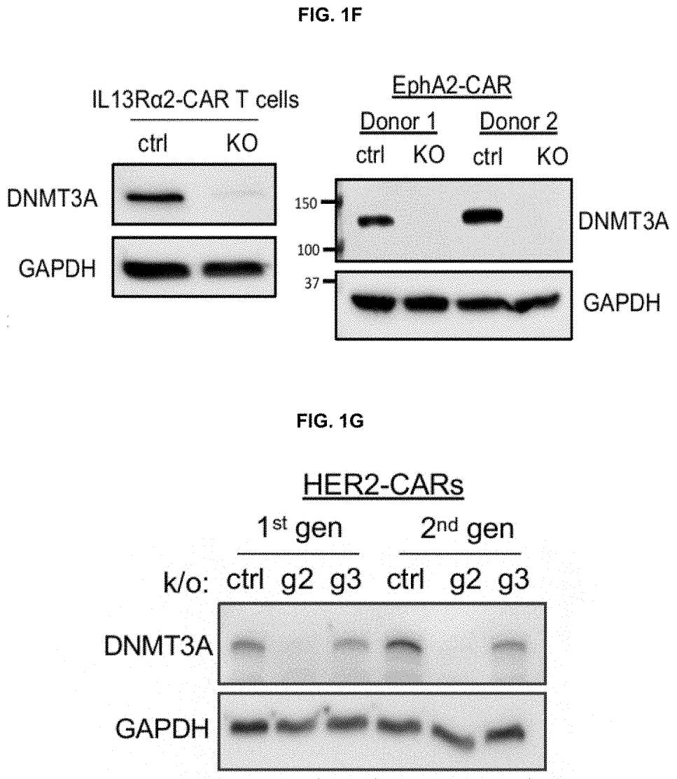

All guide RNAs were design and validated by Center of Advanced Genome Engineering (CAGE) at St. Jude Children's Research Hospital. sgRNAs were designed to target unique sites within the genome with at least 3 bp of mismatch between the target site and any other site in the genome whenever possible and common SNPs were avoided. To disrupt DNMT3A function, sgRNAs were designed targeting the DNMT3A catalytic domain located in exon 19 (FIG. 1D) (33). To mitigate the risk of any off-target mediated functional effects, an additional sgRNA (guide 3) was used that was previously validated and published (33). As an additional control, sgRNAs were designed to a sequence in mCherry that does not occur in the human genome with up to 3 bp of mismatch. To assess activity, DNMT3A sgRNAs were nucleofected as RNPs (100 μM sgRNA and 50 μM Cas9 protein using solution P3 and program FF-120) into K562, genomic DNA from transfected cells was harvested three days post-nucleofection, and NHEJ rates were determined using targeted next-generation sequencing followed by analysis with CRIS.py (63) (FIG. 1E). gRNAs with normalized NHEJ frequencies equal to or greater than 15% good candidates for cell line and animal model creation projects. sgRNAs were then transfected into T cells as described in previous section and blotted by Western to further validate activity (loss of protein) (FIGS. 1F, 1G). DNMT3A protospacer: g2-5′ CCTGCATGATGCGCGGCCCANGG 3′ (SEQ ID NO: 63), g3-5′ GCATGATGCGCGGCCCAAGGNGG 3′ (SEQ ID NO: 68) (33); mCherry protospacer: g17-5′ CAAGTAGTCGGGGATGTCGGNGG 3′ (SEQ ID NO: 64), g19-5′ AGTAGTCGGGGATGTCGGCGNGG 3′ (SEQ ID NO: 65). Primer sequences used to amplify the region of interest for NGS indicated below with the gene-specific portion underlined. The rest of the sequence is the partial Illumina adaptors:

SP230.DS.F:
(SEQ ID NO: 69)
CACTCTTTCCCTACACGACGCTCTTCCGATCTTCCCGATGACCCTGTCTT
CCCGTGC;
SP230.DS.R:
(SEQ ID NO: 70)
GTGACTGGAGTTCAGACGTGTGCTCTTCCGATCTAGGTAGAAGCCATTAG
TGAGCTGGC.

### Western Blot Analysis

Knockout efficiency was determined by Western Blot. Ctrl.k/o and DNMT3A.k/o CAR T cells were washed with PBS and lysed with RIPA buffer (Cell Signaling Technologies, Danvers, Mass.) the day of their first use in experiments. Protein quantification was performed using a BCA assay (Thermo Fisher Scientific, Waltham, Mass.), and 30 μg total protein was loaded on a 12% SDS polyacrylamide gel and then transferred to nitrocellulose membranes (BioRad, Hercules, Calif.). Membranes were blocked with 5% milk in TBS+0.1% Tween-20 (Sigma Aldrich, St Louis, Mo.) for 1 hour at room temperature. The membranes were then probed with a monoclonal rabbit human DNMT3A antibody (Cell Signaling Technologies, Danvers, Mass.) or polyclonal GAPDH antibody (Santa Cruz Biotechnologies, Dallas, Tex.) overnight at 4° C. Membranes were then washed and probed with a secondary anti-Rabbit IgG-HRP (Jackson ImmunoResearch, West Grove, Pa.) for 1 hour at room temperature. Protein expression was visualized using Femto Enhanced Chemiluminescent Substrate (Thermo Fisher Scientific, Waltham, Mass.), and images were acquired and analyzed using a Li-cor Odyssey instrument and software (Li-Cor Biosciences, Lincoln, Nebr.).

### Mouse Tumor Xenograft Models

NOD-scid IL2Rgamma^{null} mice (NSG) were obtained from breeding colonies maintained by the St. Jude Animal Resource Center. All animal experiments were performed on protocols approved by the St. Jude Institutional Animal Care and Use Committee. 8-10 week old male and female mice were used in both the glioma and osteosarcoma models. For the local LM7 model, 1×10⁶ LM7.eGFP.ffLuc cells were injected intraperitoneally on day 0. EphA2.CD28.ζ CAR T cells were stimulated with recombinant human EphA2 protein for 48 hours, rested for 24 hours, and then injected i.p. into LM7-ffLuc bearing mice on day 7. For the metastatic LM7 model, mice were injected intravenously with 2×10⁶ LM7.eGFP.ffLuc cells and on day 28 with 1×10⁶ HER2.ζ CAR T cells. The orthotopic U373 glioma model has been described previously (57). Briefly, 5×10⁴ U373.eGFP.ffLuc cells were injected through a burr-hole 1 mm anterior and 2 mm to the right of the bregma at a depth of 3 mm. 2×10⁶ IL13Rα2-CAR.CD28.ζ/IL15 T cells were injected 7 days later in the same location. For each model, CAR T cells were generated from a different healthy donor. Tumor burden was assessed weekly using bioluminescent in vivo imaging system (IVIS). Quantification of tumor burden was performed using Living Image software (Perkin Elmer, Waltham, Mass.). A region of interest (head for U373 and total body for LM7) was drawn around each mouse, and total flux was recorded in units of photons/second.

### In Vitro Functional Assays

Repeated stimulation assay: Control and DNMT3A.k/o CAR T cells were co-cultured with U373 or LM7 cells at an effector to target ratio of 2:1 in the presence of IL-15 (5 ng/mL). Seven days later, T cells were counted and replated with fresh tumor cells at the same 2:1 ratio in the presence of IL-15 (5 ng/mL). The T cells continued to be counted and stimulated with fresh tumor cells on a weekly basis until the T cells stopped killing tumor cells.

Cytokine production: At each stimulation of the repeated stimulation assay, culture supernatant was collected 24 hours after plating T cells with tumor cells for analysis of cytokine production. Cytokine production was assessed by a 14-plex human cytokine quantification kit (Millipore Sigma, Burlington, Mass.) with analysis performed using a Luminex FlexMap 3D instrument and software (Luminex Corporation, Austin, Tex.).

Cytotoxicity assay: A CellTiter96® AQueous One Solution Cell Proliferation Assay (Promega, Madison, Wis.) was utilized to assess CAR T cell cytotoxicity as previously described (35). Briefly, tumor cells were incubated with varying amounts of CAR T cells to assess cytotoxicity at a range of E:T ratios. 24 hours later, the media and T cells were removed, and the remaining tumor cells were quantified with MTS reagent. Media only and tumor only served as controls to assess percent cytotoxicity. MTS assays were performed using unstimulated CAR T cells (1^{st} stimulation) and CAR T cells that had previously been exposed to tumor cells three times (4^{th} stimulation).

CAR T cell antigen dependence assay: Survival of serially-stimulated DNMT3A.k/o CAR T cells in the absence of tumor was assessed by positive selection of CD3+ co-culture cells (Miltenyi Biotec, Bergisch Gladbach, Germany) followed by 7 day culture in the absence of tumor cells+/−IL-15. Live and dead cells were enumerated by flow cytometry using an eFluor 520 live/dead stain (Thermo Fisher Scientific, Waltham, Mass.) and annexin V APC (BD Biosciences, Franklin Lakes, N.J.).

### Flow Cytometry and FACS Sorting

Immunophenotyping: Samples were phenotyped for cell surface antigens prior to and post-co-culture. Live cell discrimination was performed with an eFluor 506 fixable viability dye. The following antibodies were used in phenotypic analysis: CD3 BV605, CD4 APC-Cy7, CD45RO BV421, PerCP-Cy5.5 CCR7 (BD Biosciences, Franklin Lakes, N.J.), CD8 Alexa-488. Isotype-matched controls were run for each experiment. Surface staining was performed prior to fixation, permeabilization, and intracellular staining using the eBioscience Transcription Factor Staining Buffer Set (Thermo Fisher Scientific, Waltham, Mass.). Samples were acquired on a BD FACS Lyric, and list mode files were analyzed using FlowJo ver 10.5.3 (BD Biosciences, Franklin Lakes, N.J.).

FACS sorting for WGBS: After the 4^{th} stimulation, Ctrl.k/o and DNMT3A.k/o CAR T cells were FACS purified based on live CD8⁺ phenotyping. DNA was extracted and bisulfite converted (Zymo EZ DNA methylation-direct kit) which converted all unmethylated cytosines to uracils while protecting the methylated cytosines from deamination. Bisulfite-modified DNA libraries were sequenced using Illumina HiSeq and sequencing data were mapped to the HG19 human genome. Differences in CpG methylation across the genome were analyzed using IGV software.

T cell subset isolation: For experiments using purified cell populations, PBMCs from fresh blood from healthy consented donors were isolated by centrifugation in BD Vacutainer CPT tubes (BD Biosciences, Franklin Lakes, N.J.) and enriched prior to sorting. Following CD8 enrichment, naïve and memory CD8 T cell compartments were FACS purified. Cells were stained in sterile PBS containing 4% FBS with the same antibodies described above. Individual cell populations were sorted using a BD FACS Aria II (BD Biosciences, Franklin Lakes, N.J.). Naïve CD8 T cells were defined as live C8⁺, CCR7⁺, and CD45RO⁻ cells. Of note, the naïve compartment included Tscm phenotyped (CD95⁺) cells. The memory CD8 T cell compartment was sorted based on live CD8′, CD45RO⁺ expression. The memory compartment included Tem (CCR7⁻) and Tcm (CCR7⁺) CD8 T cells. Sorted cells were checked for purity and determined to be at least 95% of the desired phenotype.

Transduction efficiencies: Efficiency of retroviral transduction was determined by flow cytometry using the following antibodies: IL13Rα2 CAR: recombinant IL13Rα2-F_{c} fusion protein (R&D systems, Minneapolis, Minn.) followed by anti-human IgG F_{c} PE (Southern Biotech, Birmingham, Ala.); HER2 CAR: anti-mouse F_{ab} Alexa-647 (Jackson ImmunoResearch, West Grove, Pa.); EphA2 CAR: anti-human CD19 PE (Beckman Coulter, Brea, Calif.); secretable IL-15: anti-human NGFR BV421 (BD Biosciences, Franklin Lakes, N.J.).

### Bisulfite Sequencing Methylation Profiling and Data Analysis

Genome DNA was isolated from FACS purified T cells and processed as previously described (64). Briefly, DNA was extracted from the sorted cells using a DNA-extraction kit (Qiagen) and then bisulfite treated using an EZ DNA methylation kit (Zymo Research), or an EZ DNA methylation-direct kit (Zymo Research). The bisulfite-modified DNA-sequencing library was generated using the EpiGnome™ kit (Epicentre) per the manufacturer's instructions and then sequenced using an Illumina Hiseq or Novaseq instrument. Sequencing data were aligned to the hg19 genome using BSMAP2.74 (65). Differentially methylated regions (DMRs) were identified using Bioconductor package DSS (66). Statistical test of differentially methylated locus (DML) was first performed using DMLtest function (smoothing=TRUE) in DSS, the results were then used to detect differentially methylated regions using CallDMR function in DSS, the p value threshold for calling DMR is 0.01. The minimum length for defining a DMR was set to 50 bps and the minimum number of CpG sites for defining a DMR is 3. The GSEA analyses were performed using 108 DNMT3A-target transcription factors as gene signature on gene expression data from GSE23321 (67). Ingenuity pathway analysis was performed using DNMT3A-targeted genes.

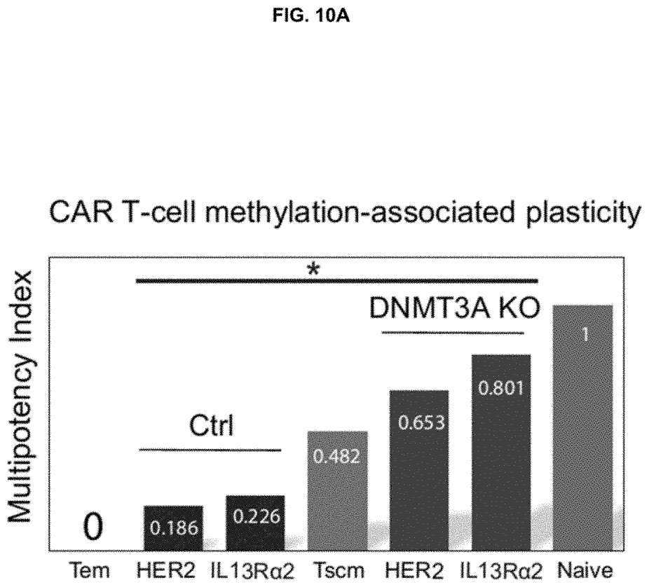

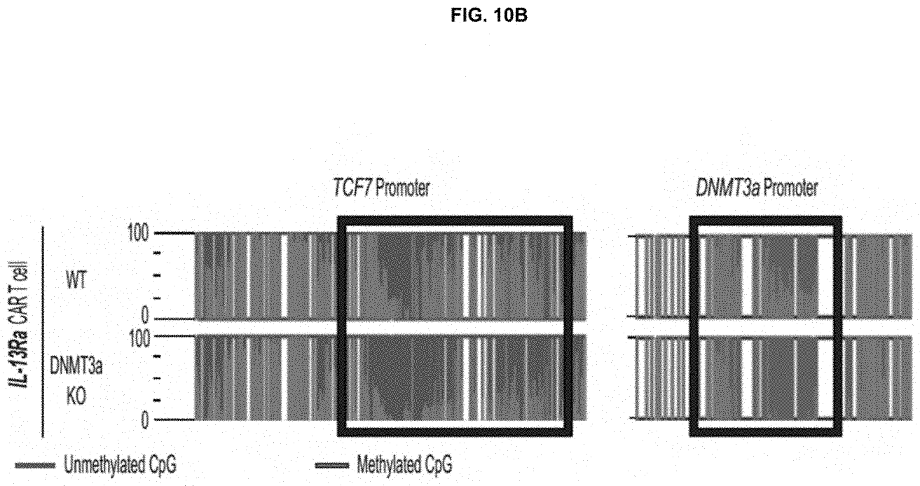

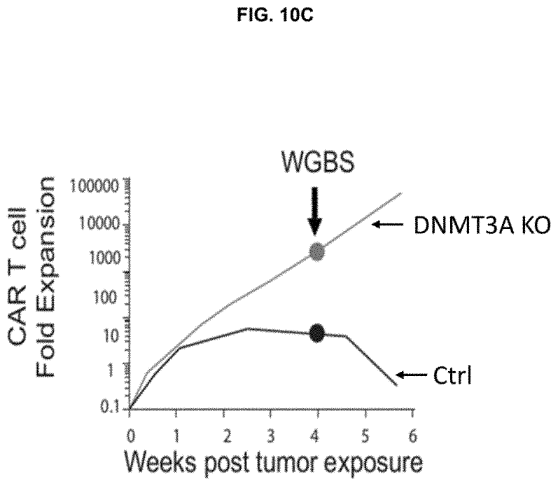

A human CD8+ T cell DNA methylation has been established based multipotency index using a machine learning approach. Briefly, to identify the methylation state of CpG sites associated with the T cell multipotent potential, a supervised analysis was performed between the methylomes from two naive and four HIV-specific CD8 T cells (methylation difference >=0.4 and FDR<=0.01). This analysis results in identification of 245 CpGs sites that were hypomethylated in naive CD8+ T cells compared to HIV-specific CD8+ T cells. This set of the CpGs was then used as an input to the one-class logistic regression to calculate the multipotency signature using just the HIV-specific and naïve CD8+ T cells (Training data sets) (68) (69). Once the signature was obtained, it was then applied to the naïve CD8+ T cells, Tscm, and Tem from healthy donors, and CAR-T cell methylomes (test data sets). The score was calculated as the dot product between the DNA methylation value and the signature. The score was subsequently converted to the [0, 1] range. Data sets with multipotency indices closer to 1 were more similar to naive cells (FIG. 10A).

### CHANGE-seq

Genomic DNA from T cells was isolated using Gentra Puregene Kit (Qiagen) according to manufacturer's instructions. Purified genomic DNA was tagmented with a custom Tn5-transposome to an average length of 400 bp, followed by gap repair with Kapa HiFi HotStart Uracil+ DNA Polymerase (KAPA Biosystems) and Taq DNA ligase (NEB). Gap-repaired tagmented DNA was treated with USER enzyme (NEB) and T4 polynucleotide kinase (NEB). Intramolecular circularization of the DNA was performed with T4 DNA ligase (NEB) and residual linear DNA was degraded by a cocktail of exonucleases containing Plasmid-Safe ATP-dependent DNase (Lucigen), Lambda exonuclease (NEB) and Exonuclease I (NEB). In vitro cleavage reactions were performed with 125 ng of exonuclease-treated circularized DNA, 90 nM of SpCas9 protein (NEB), Cas9 nuclease buffer (NEB) and 270 nM of sgRNA, in a 50 μL volume. Cleaved products were A-tailed, ligated with a hairpin adaptor (NEB), treated with USER enzyme (NEB) and amplified by PCR with barcoded universal primers NEBNext Multiplex Oligos for Illumina (NEB), using Kapa HiFi Polymerase (KAPA Biosystems). Libraries were quantified by qPCR (KAPA Biosystems) and sequenced with 150 bp paired-end reads on an Illumina NextSeq instrument. CHANGE-seq data analyses were performed using open-source CIRCLE-seq analysis software (https://github.com/tsailabSJ/circleseq).

### Histology and Immunohistochemistry

Formalin fixed tissues were decalcified in formic acid and were processed and embedded in paraffin by standard techniques, sectioned at 4 μm, mounted on positively charged glass slides (Superfrost Plus; Thermo Fisher Scientific, Waltham, Mass.), and dried at 60° C. for 20 min. Tissue sections were then stained with hematoxylin and eosin (HE) or subjected to immunohistochemical (IHC) staining for CD3+ T cells using a rabbit polyclonal antibody raised against CD3 diluted 1:1,000 (cat #AB152, Santa Cruz Biotechnology, Dallas, Tex.). Tissue sections underwent antigen retrieval in prediluted Cell Conditioning Solution (CC1; Cat #950-124; Ventana Medical Systems, Indianapolis, Ind.) for 32 min on a Discovery Ultra immunostainer (Ventana Medical Systems, Tucson, Ariz.). Binding of primary antibodies was detected using OmniMap anti-Rabbit (#760-4311; Ventana Medical Systems), with DISCOVERY ChromoMap DAB Kit (Ventana Medical Systems) as chromogenic substrate. Stained sections were examined by a pathologist blinded to the experimental group assignments.

### Example 1. Generation of Gene Edited CAR T Cells

Peripheral blood mononuclear cells (PBMCs) were plated onto αCD3 and CD28 coated plates on Day 0. On Day 2, activated T cells were transduced with retroviral vectors encoding a 1^{st} generation or 2^{nd} generation HER2-specific chimeric antigen receptor (HER2-CAR), a 2^{nd} generation IL13Rα2-CAR, or a 2^{nd} generation EphA2-CAR to generate CAR T cells. The scheme of HER2, IL13Rα2, and EphA2-specific CAR retroviral vectors is shown in FIG. 1C. The DNMT3A gene was knocked out in the CAR T cells (CAR. DNMT3A.k/o-T cells) by electroporating the T cells with Cas9 (MacoLabs, Berkeley)/DNMT3A-specific sgRNA complexes (referred to as RNP complexes) on Day 3. Two DNMT3A-specific sgRNA sequences (guide 2 and guide 3) were designed by the Center for Advanced Genome Engineering (CAGE) core facility at St. Jude which target the catalytic domain (exon 19) (33) of DNMT3A (FIGS. 1D, 1E). The DNMT3A-specific sgRNA guide 2 contains the following nucleotide sequence: CCTGCATGATGCGCGGCCCANGG (SEQ ID NO: 63) and guide 3 contains the following nucleotide sequence GCATGATGCGCGGCCCAAGGNGG (SEQ ID NO: 68). As controls, CAR T cells were electroporated with Cas9 sgRNA complexes specific for an irrelevant gene (mCherry g17 (SEQ ID NO: 64); Ctrl.k/o CAR T cells). Cells were grown for additional 9 days with IL-7 and IL-15 added to the medium every 2-3 days. On Day 12, cells were collected and used for in vitro and/or in vivo testing. The procedure for the generation of gene edited CAR T cells is illustrated in FIG. 1A. All CAR T cell constructs used in the study were generated and tested at Baylor College of Medicine. This method resulted in highly efficient knockout of DNMT3A irrespective of CAR construct (FIGS. 1F, 1G) with no effect on CAR cell surface expression or phenotype and with limited off-target gene disruptions.

### Example 2. Repeat Stimulation Experiment

To evaluate the ability of DNMT3A.k/o CAR T cells to proliferate after repeat stimulations, DNMT3A.k/o CAR T cells or Ctrl.k/o CAR T cells were exposed every 7 days to fresh tumor cells in the absence or presence of IL-15. The procedure for the repeat stimulation experiment is outlined in FIG. 1B. Briefly, 1×10⁶ CAR T cells were co-cultured with 0.5×10⁶ tumor cells (U373 cells from ATCC) at a 2:1 E:T ratio in the presence or absence of IL-15 (stimulation 1). Recombinant IL-15 (PeproTech, Cat #200-15) was used. 7 days later, the T cells were harvested, counted and 1×10⁶ T cells were re-plated on fresh 0.5×10⁶ tumor cells in the presence or absence of IL-15 (stimulation 2). This procedure was repeated weekly until the T cells stopped killing tumor cells, or stopped expanding and/or started dying.

### Example 3. Proliferation of DNMT3A Deleted HER2-CAR T Cells

DNMT3A deleted HER2-CAR T cells (HER2-CAR.DNMT3A.k/o-T cells) were generated expressing a 1^{st} generation HER2-CAR (HER2-CAR.ζ) and a 2^{nd} generation HER2-CAR (HER2-CAR.CD28.ζ) as described in Example 1. As controls, HER2-CAR T cells were electroporated with Cas9 sgRNA complexes specific for an irrelevant gene (mCherry g17; HER2-CAR.Ctrl.k/o-T cells). The functional domains in the HER2-CARs and their corresponding protein and DNA sequences are provided in Table 1.

TABLE 1
HER2-CAR domains and sequences

| | | Protein | DNA |
|---|---|---|---|
| | | sequence | sequence |
| | | SEQ | SEQ |
| CAR or scFv name | Domain name | ID NO | ID NO |
| Anti-HER2 scFv | Full-length | 1 | 2 |
| (scFvFRP5 + | leader sequence | 15 | 16 |
| leader) | scFvFRP5 | 17 | 18 |
| HER2-specific | Full-length | 3 | 4 |
| 1^{st} generation | leader sequence | 15 | 16 |
| CAR (HER2- | scFvFRP5 | 17 | 18 |
| CAR.ζ) | short hinge | 19 | 20 |
| | linker domain | 21 | 22 |
| | (including hinge) | | |
| | CD3 zeta transmembrane | 23 | 24 |
| | domain | | |
| | CD3 zeta signaling domain | 25 | 26 |
| HER2-specific | Full-length | 5 | 6 |
| 2^{nd} generation | leader sequence | 15 | 16 |
| CAR (HER2- | scFvFRP5 | 17 | 18 |
| CAR.CD28.ζ) | short hinge | 19 | 20 |
| | linker domain | 21 | 22 |
| | (including hinge) | | |
| | CD28 transmembrane and | 35 | 36 |
| | co-stimulatory domain | | |
| | CD3 zeta signaling domain | 25 | 26 |

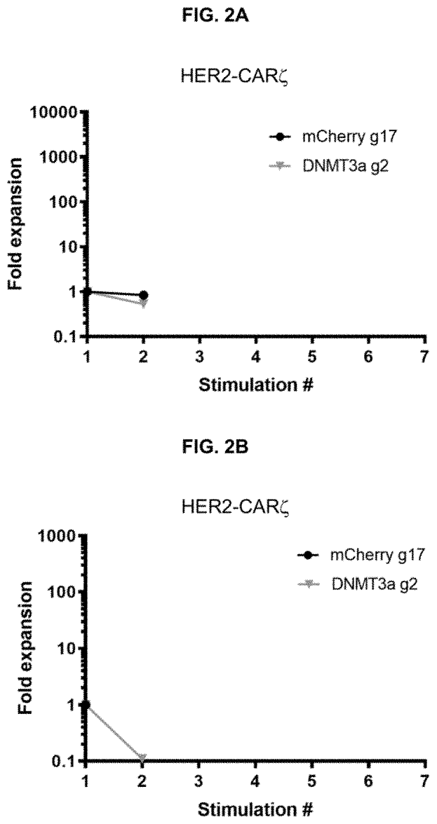

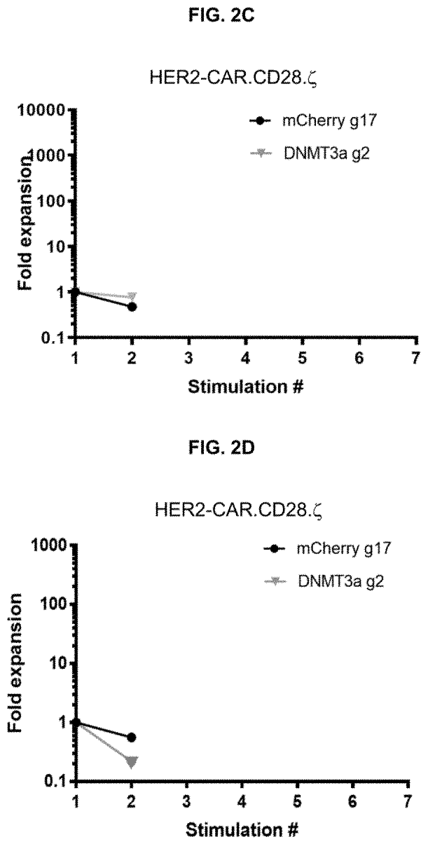

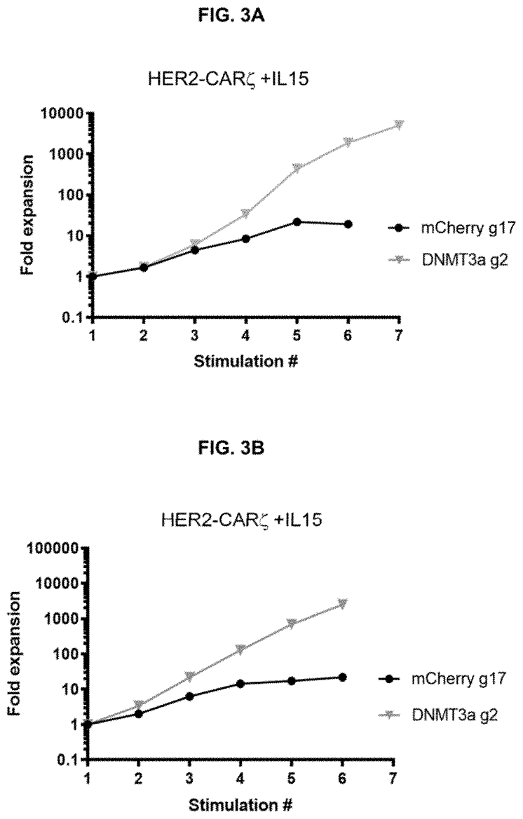

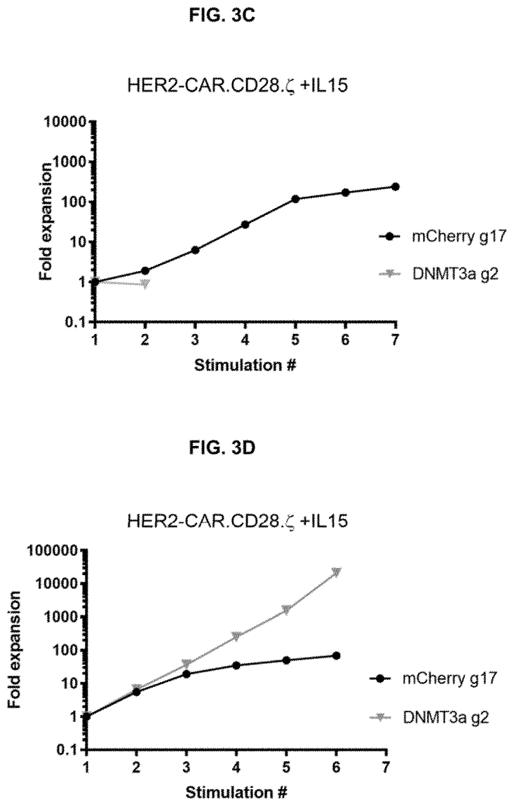

To evaluate the ability of HER2-CAR.DNMT3A.k/o-T cells to proliferate after repeat stimulations, the repeat stimulation experiment was performed as described in Example 2. This experiment was conducted independently for 2 donors. For both donors, HER2-CAR.DNMT3A.k/o-T cells and HER2-CAR.Ctrl.k/o-T cells in the absence of IL15 did not expand (FIGS. 2A-2D). In the presence of IL-15, DNMT3A.k/o-T cells expressing a 1^{st} generation HER2-CAR (HER2-CAR.ζ) expanded >10 to 100-fold better than HER2-CAR.ζ.Ctrl.k/o-T cells (FIGS. 3A-3B). For DNMT3A.k/o-T cells expressing a 2^{nd} generation CAR (HER2-CAR.CD28.ζ.DNMT3A.k/o-T cells), greater expansion was observed in 1 out 2 donors (FIGS. 3C-3D).

### Example 4. Antitumor Ability of DNMT3A Deleted HER2-CAR T Cells

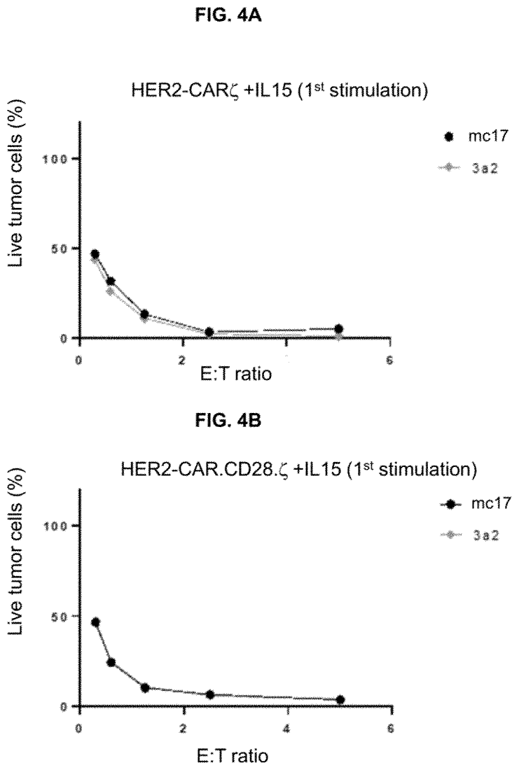

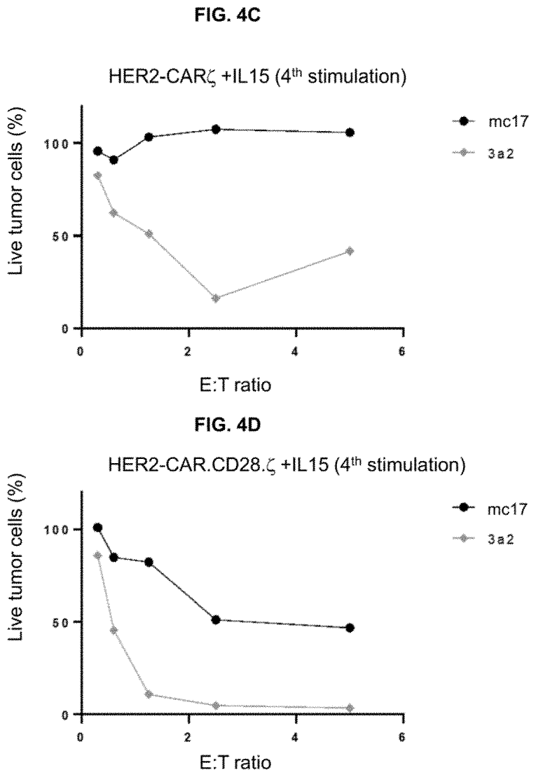

To evaluate the antitumor ability of the HER2-CAR.DNMT3A.k/o-T cells, tumor cell viability was examined. 1×10⁶ gene edited CAR T cells were co-cultured with tumor cells at varying E:T ratios (0.3-5) in the presence of IL-15. The repeat stimulation procedure was applied as described in Example 2. The experiment was performed with cells from one donor. In addition to improved proliferation, HER2-CAR.ζ.DNMT3A.k/o- and HER2-CAR.CD28.ζ.DNMT3A.k/o-T cells also retained their ability to kill HER2+ tumor cells in contrast to CAR Ctrl.k/o T cells after the 4^{th} stimulation (FIGS. 4A-4D).

### Example 5. Proliferation and Antitumor Activity of DNMT3A Deleted IL13Rα2-CAR T Cells

DNMT3A deleted IL13Rα2-CAR T cells (IL13Rα2-CAR.DNMT3A.k/o-T cells) were generated expressing a 2^{nd} generation IL13Rα2-CAR (IL13Rα2-CAR.CD28.ζ) as described in Example 1. As controls, IL13Rα2-CAR T cells were electroporated with Cas9 sgRNA complexes specific for an irrelevant gene (mCherry g17; IL13Rα2-CAR.Ctrl.k/o-T cells). The functional domains in the IL13Rα2-CARs and their corresponding protein and DNA sequences are provided in Table 2.

TABLE 2
IL13Rα2-CAR domains and sequences

| | | Protein | DNA |
|---|---|---|---|
| | | sequence | sequence |
| | | SEQ | SEQ |
| CAR or scFv name | Domain name | ID NO | ID NO |
| Anti-IL13Rα2 scFv | Full-length | 7 | 8 |
| (scFv47 + leader) | leader sequence | 15 | 16 |
| | scFv47 | 29 | 30 |
| IL13Rα2-specific | Full-length | 9 | 10 |
| 2^{nd} generation | leader sequence | 15 | 16 |
| CAR (IL13Rα2- | scFv47 | 29 | 30 |
| CAR.CD28.ζ) | short hinge | 19 | 20 |
| | linker domain | 21 | 22 |
| | (including hinge) | | |
| | CD28 transmembrane | 35 | 36 |
| | domain and co- | | |
| | stimulatory domain | | |
| | CD3 zeta signaling domain | 25 | 26 |
| scFv47.SH.delta | Full-length | 11 | 12 |
| CAR | leader sequence | 15 | 16 |
| | scFv47 | 29 | 30 |
| | short hinge | 19 | 20 |
| | linker domain | 21 | 22 |
| | (including hinge) | | |
| | CD28 transmembrane | 31 | 32 |
| | domain | | |
| | CD28 co-stimulatory | 66 | 67 |
| | domain (partial) | | |

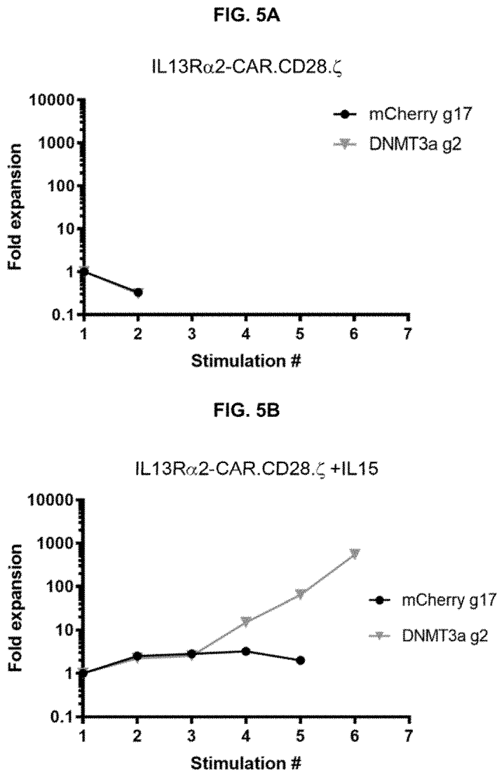

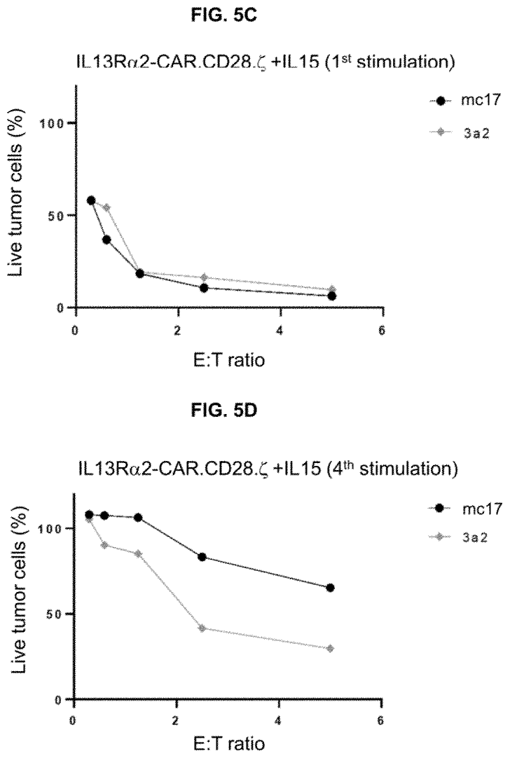

The proliferation and antitumor activity of IL13Rα2-CAR.DNMT3A.k/o-T cells were evaluated similarly as described for the HER2-CAR.DNMT3A.k/o-T cells above. IL13Rα2-CAR.CD28.ζ.DNMT3A.k/o-T cells expanded >10 to 100-fold better than IL13Rα2-CAR.ζ.Ctrl.k/o-T cells in the presence of IL-15 (FIG. 5B). No expansion was observed in the absence of IL-15 (FIG. 5A). IL13Rα2-CAR.CD28.ζ.DNMT3A.k/o T cells retained their ability to kill IL13Rα2+ tumor cells after the 4^{th} stimulation (FIGS. 5C-5D).

### Example 6. Whole-Genome DNA Methylation Profiling of DNMT3A Deleted CAR T cells

To decipher the mechanism of improved effector function of DNMT3A.k/o CAR T cells whole-genome DNA methylation profiling was performed. A DNA methylation-based T cell multipotency index was used to compare the epigenetic state of DNMT3A.k/o and Ctrl.k/o CAR T cells (WT CAR T cells) (FIG. 10A). Ctrl.k/o CAR T cells clustered with differentiated effector memory T cells (Tems), and DNMT3A.k/o CAR T cells had characteristics of naïve T cells and long-lived stem cell-like memory T cells (Tscms). The methylation status of the DNMT3A promoter was also studied. The promoter of the transcription factor Tcfl (encoded by the gene TCF7), which is critical for cell self-renewal, is demethylated in DNMT3A.k/o CAR T cells similar to the DNMT3A promoter locus, providing additional mechanistic insight (FIG. 10B).

### Example 7. Antigen Dependency of DNMT3A Deleted CAR T Cells

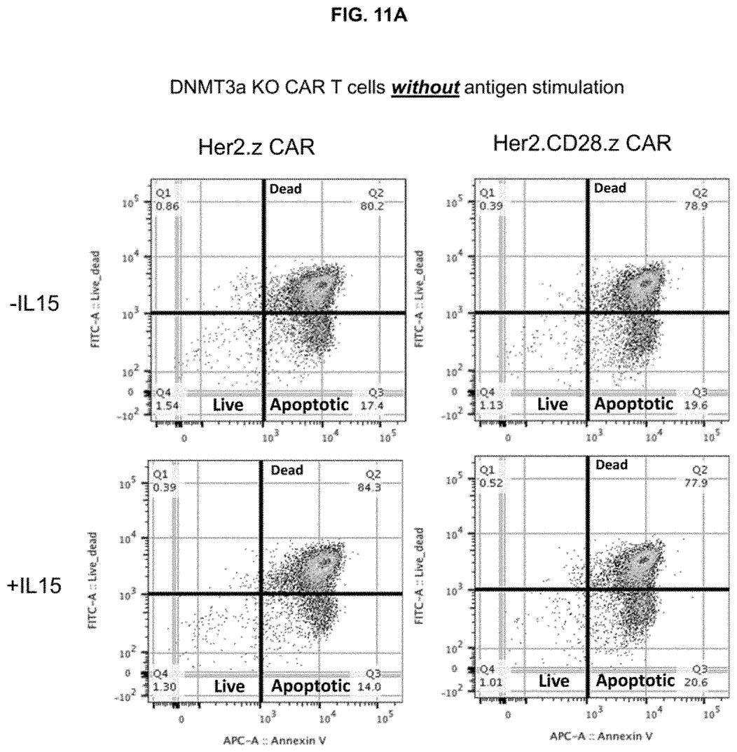

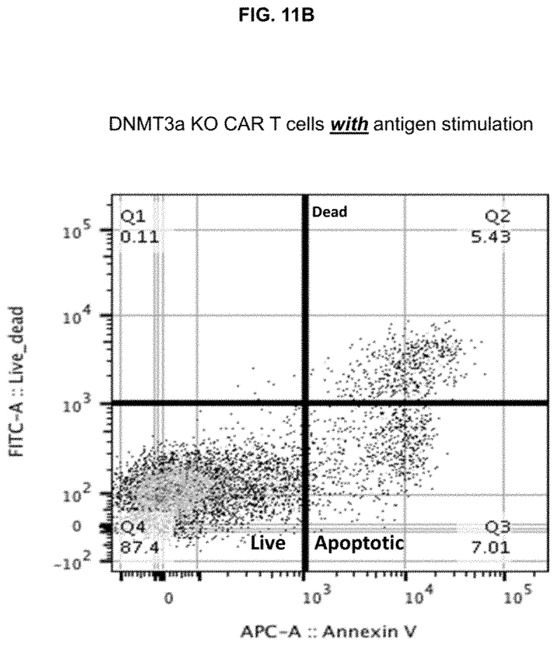

To test if DNMT3A.k/o CAR T cell expansion is antigen dependent, HER2-CAR.DNMT3A.k/o T cells were cultured after 5^{th} stimulation without tumor cells but in the present or absence of IL-15. At day 8, DNMT3A.k/o CAR T cells were collected and fluorescence-activated cell sorting (FACS) analysis was performed to determine CAR T cell viability. All CAR T cells that were grown without antigen stimulation (without tumor cells) were dead or apoptotic after 8 days of culture in the present or absence of IL-15 (FIG. 11A). In contract, DNMT3A.k/o CAR T cell in the presence of antigen stimulation (with tumor cells) were alive after 8 days of culture (FIG. 11B). The results show that the expansion of DNMT3A.k/o CAR T cells is antigen dependent.

### Example 8. Evaluation of DNMT3A Deleted EphA2-CAR T Cells

EphA2 (4H5) specific 2^{nd} generation CAR (EphA2-CAR.CD28.ζ) T cells expressing IL15 were generated and gene edited with Cas9 guide RNAs targeting DNMT3A or mCherry as described in Example 1. The functional domains in the EphA2-CAR and their corresponding protein and DNA sequences are provided in Table 3.

TABLE 3
EphA2-CAR domains and sequences

| | | Protein | DNA |
|---|---|---|---|
| | | sequence | sequence |
| | | SEQ | SEQ |
| CAR or scFv name | Domain name | ID NO | ID NO |
| EphA2 (4H5) | Full-length | 13 | 14 |
| specific | Leader Sequence | 15 | 37 |
| 2^{nd} generation | scFv 4H5 | 38 | 39 |
| CAR (EphA2- | IgG1 Short Hinge | 40 | 41 |
| CAR.CD28.ζ) | linker domain | 21 | 42 |
| | (including hinge) | | |
| | CD28 transmembrane and | 35 | 43 |
| | co-stimulatory domain | | |
| | CD3 zeta signaling domain | 25 | 44 |
| | T2A sequence | 45 | 46 |
| | Truncated CD19 | 47 | 48 |

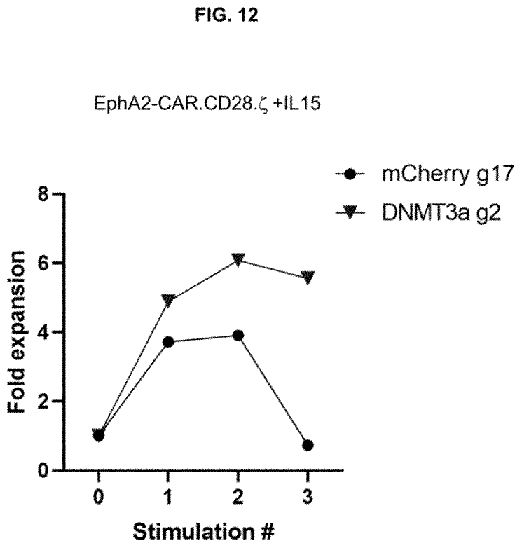

To test their expansion and persistence, the repeat stimulation experiment was performed as described in FIG. 1B. In the presence of IL-15, DNMT3A.k/o-T cells expressing a 2^{nd} generation EphA2-CAR expanded better than EphA2-CAR.Ctrl.k/o-T cells (FIG. 12).

### Example 9. Further Evaluation of DNMT3A Deleted CAR T Cells

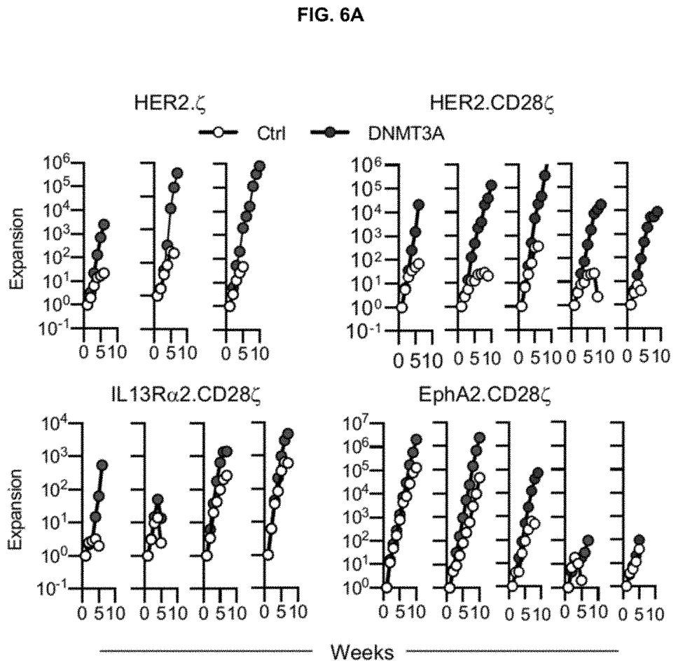

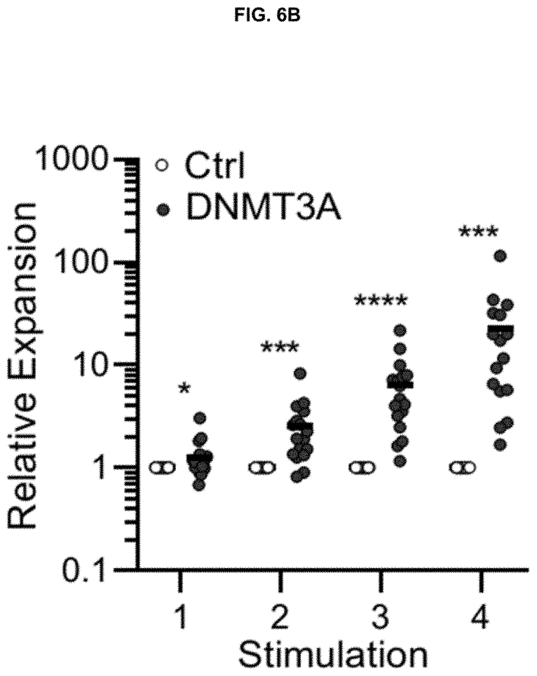

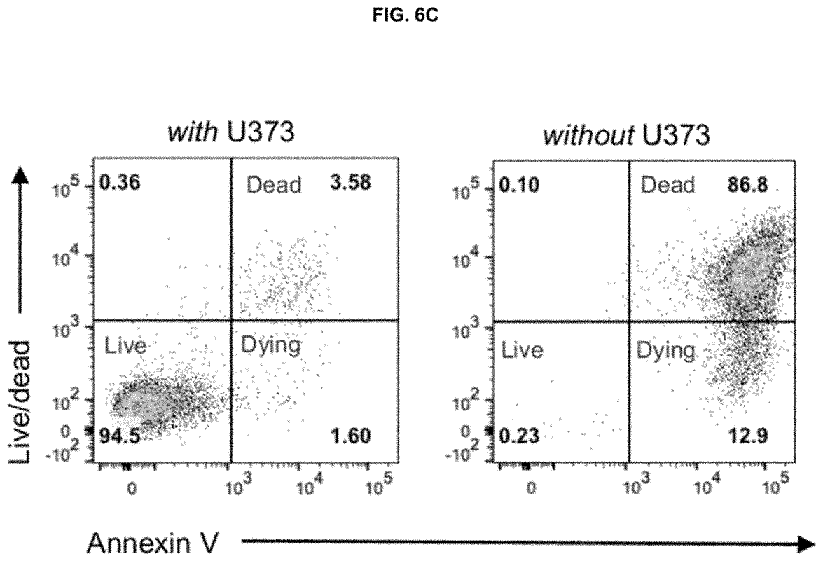

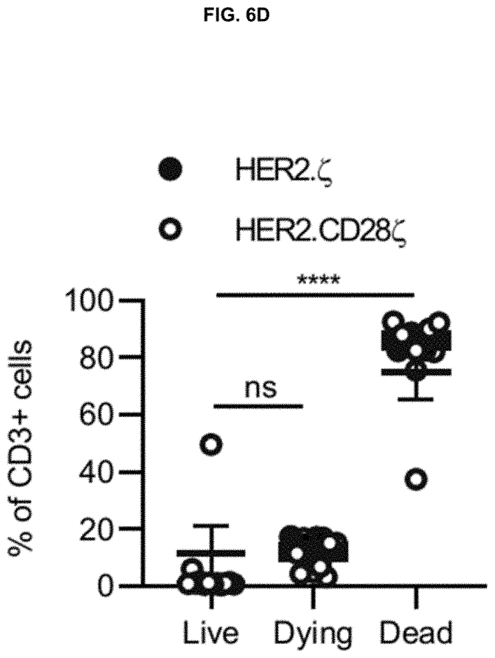

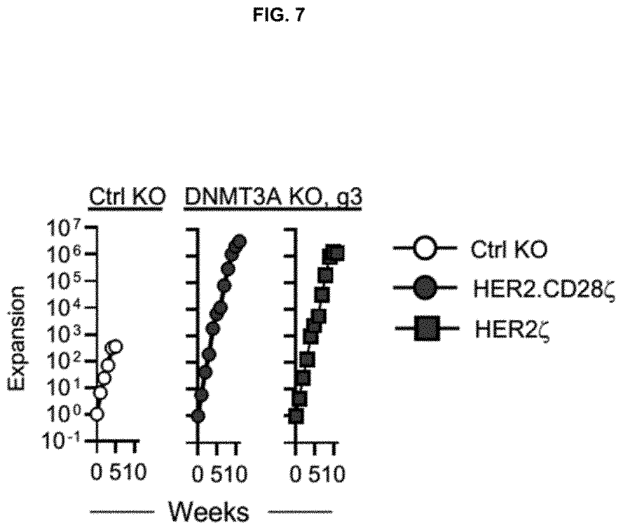

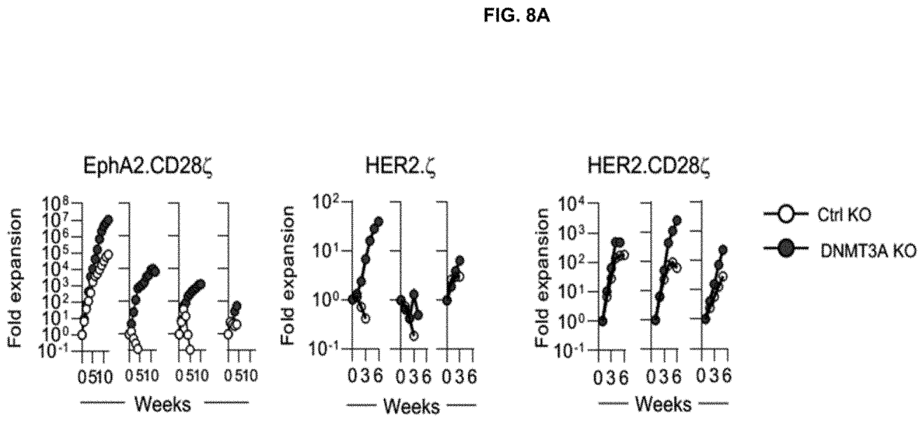

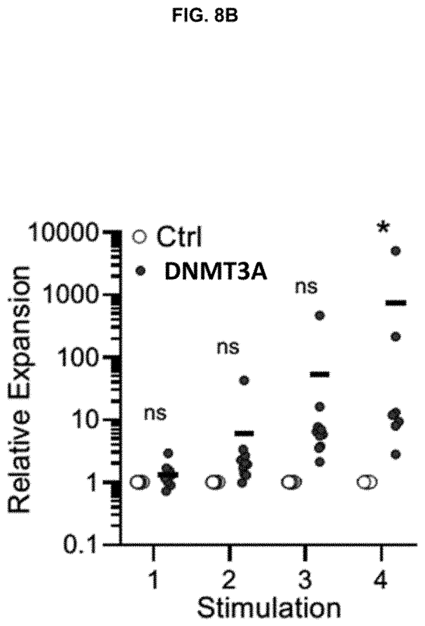

Regardless of CAR T cell population, DNMT3A.k/o CAR T cell expansion was significantly greater than Ctrl.k/o CAR T cells (FIGS. 6A, 6B) with an average 1.3-fold difference after the 1^{st}, 2.5-fold after the 2^{nd}, 6.4-fold after the 3^{rd}, and 22.6-fold after the 4^{th} stimulation, respectively (FIG. 6B). This difference was strictly antigen dependent, since culturing DNMT3A.k/o CAR T cells without tumor cells for 7 days resulted in a mean 93.2% of dead/dying DNMT3A.k/o CART cells (FIGS. 6C, 6D). To confirm the observed benefit was DNMT3A specific, the findings were confirmed using a second guide (g3) (FIG. 7). Lastly, experiments were performed with an osteosarcoma tumor cell line (LM7, positive for HER2 and EphA2) and demonstrated that DNMT3A.k/o CAR T cells also expanded to a greater extent than Ctrl.k/o CAR T cells (FIGS. 8A, 8B).

### Example 10. Cytokine Production and Cytolytic Activity of DNMT3A Deleted CAR T Cells

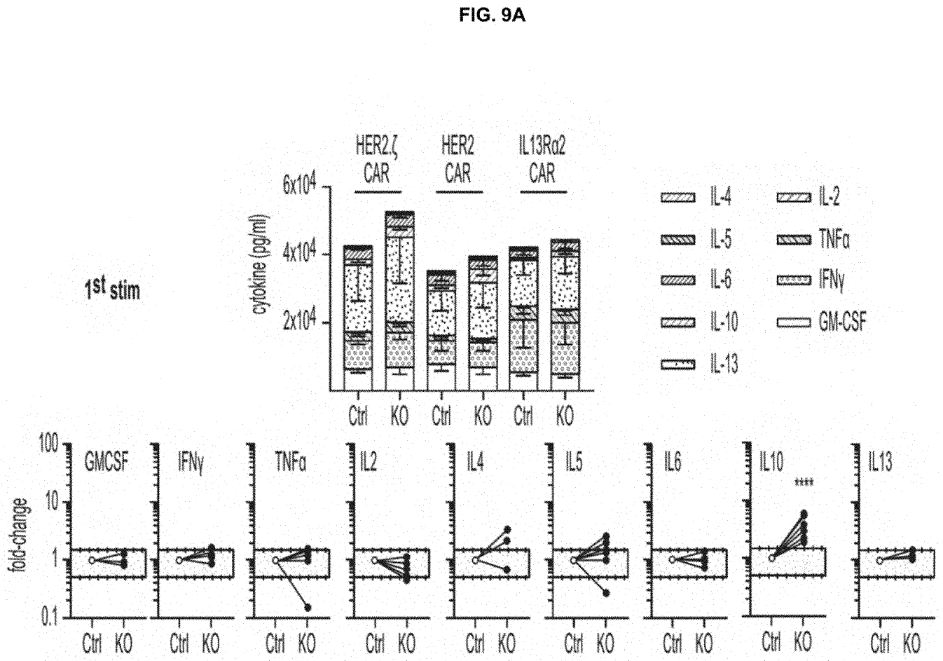

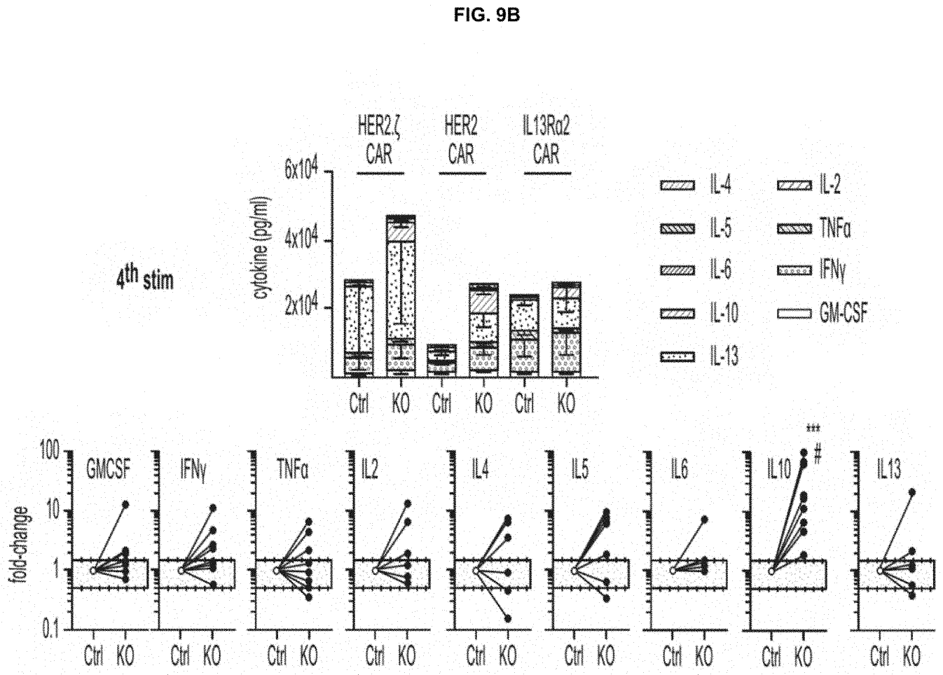

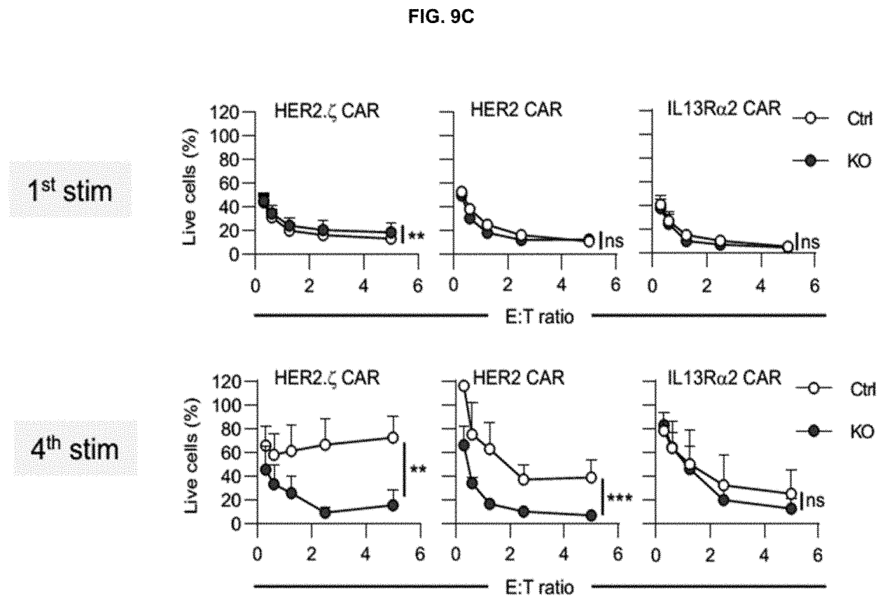

It was demonstrated in the Examples above that DNMT3A.k/o CAR T cells have a significantly greater capability to expand in the presence of tumor cells compared to Ctrl.k/o CAR T cells. In this Example, the DNMT3A deleted CAR T cells' ability to produce cytokines was assessed and their cytolytic activity was quantified. DNMT3A.k/o CAR T cells and control HER2-CAR.ζ−, HER2. CAR-CD28.ζ−, or IL13Rα2-CAR.CD28.ζ T cells were cultured with U373 cells and after 24 hours the concentration of Th1 (GM-CSF, IFNγ, TNFγ, IL-2) and Th2 (IL-4, IL-5, IL-6, IL-10, IL-13) cytokines was determined by multiplex analysis (FIG. 9A). Comparison of control and DNMT3A.k/o CAR T cells after one round of stimulation revealed that all cytokines analyzed were secreted at equivalent levels, except IL-10 (mean 3.3-fold difference for Ctrl.k/o vs DNMT3A.k/o CAR T cells) (FIG. 9A). The same analysis was repeated with Ctrl.k/o and DNMT3A.k/o CAR T cells after the 4^{th} stimulation. While there was greater variability in cytokine secretion among donors after the 4^{th} stimulation, the only differentially expressed cytokine remained IL-10 (FIG. 9B). IL-10 has been previously shown to stimulate proliferation of activated CD8 T cells and augment the survival and function of memory-type T cell effectors (36). Therefore, the upregulated secretion of IL-10 in DNMT3A.k/o CAR T cells appears to be coupled to the retained proliferative capacity of the DNMT3A deficient T cells, and may suggest that epigenetic reprogramming of T cells selectively enriches for the survival of a memory population. The cytolytic capacity of Ctrl.k/o- and DNMT3A.k/o CAR T cells was also determined at the 1^{st} and 4^{th} stimulation. DNMT3A knockout allowed CAR T cells to retain the ability to kill target cells at later stimulations compared to Ctrl.k/o CAR T cells (FIG. 9C). Collectively, the data demonstrate that deletion of DNMT3A preserves CAR T cell proliferative capacity and effector functions during extended in vitro tumor challenge. Additionally, in the absence of antigen (tumor cells) DNMT3A.k/o CAR T cells undergo the normal process of T cell contraction.

### Example 11. In Vivo Testing of Intraperitoneally Administered DNMT3A Deleted CAR T Cells

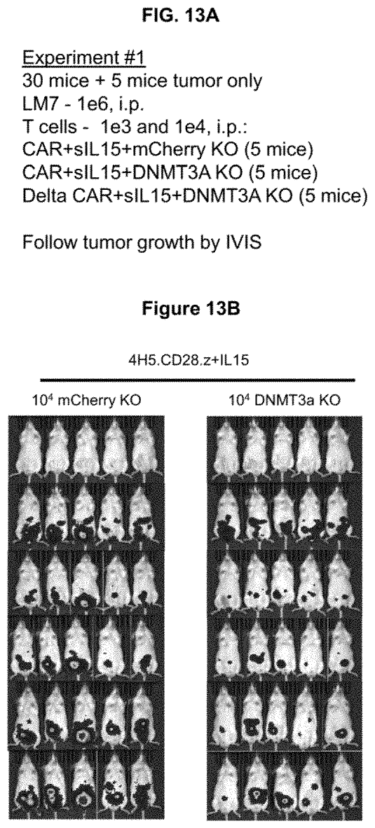

An intraperitoneal LM7 xenograft model was utilized for the in vivo testing of DNMT3A deleted CAR T cells (FIG. 13A). The DNMT3A deleted CAR T cells were also engineered to express IL15. 8-week old female NSG mice were injected intraperitoneally (i.p.) with 10⁶ LM7 tumor cells, which were genetically modified to express firefly luciferase (ffluc) to enable non-invasive bioluminescence imaging to track tumor cells. On Day 7, mice received a single i.p. injection of 10³ or 10⁴ DNMT3A.k/o or Ctrl.k/o CAR T cells. Each treatment group consisted of 5 mice. Tumor burden was monitored by bioluminescence imaging using an in vivo imaging system (IVIS) Spectrum imager. EphA2-specific DNMT3A.k/o CAR T cells showed better anti-tumor activity and tumor growth control when compared to Ctrl.k/o CAR T cells (FIG. 13B).

### Example 12. In Vivo Testing of Intracranially Administered DNMT3A Deleted CAR T Cells

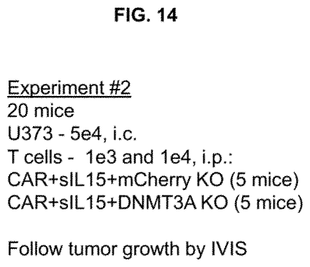

An orthotopic U373 glioma xenograft mouse model is utilized in which T cells are directly injected into tumors (FIG. 14). The model allows for serial bioluminescence imaging since U373 are genetically modified to express an eGFP.ffLuc fusion protein (U373.eGFP.ffLuc). On Day 0, U373.eGFP.ffLuc cells are injected stereotactically into brains of NSG mice. On Day 7, mice receive a single intracranial (i.c.) injection of 10³ or 10⁴ DNMT3A.k/o or Ctrl.k/o CAR T cells expressing HER2-CAR.CD28.ζ or IL13Rα2-CAR.CD28.ζ. Each treatment group consists of 5 mice. Tumor burden is monitored by bioluminescence imaging.

### Example 13. DNMT3A Deletion Enhances the Therapeutic Efficacy of CAR T Cells in Preclinical Solid Tumor Models

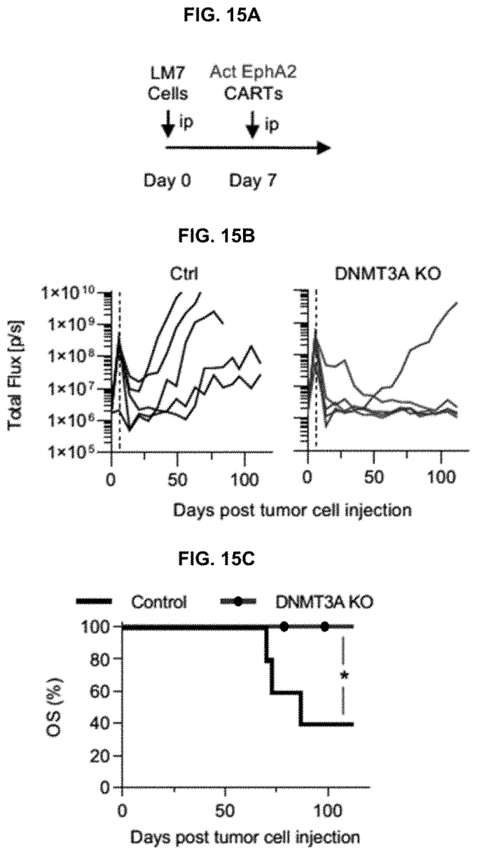

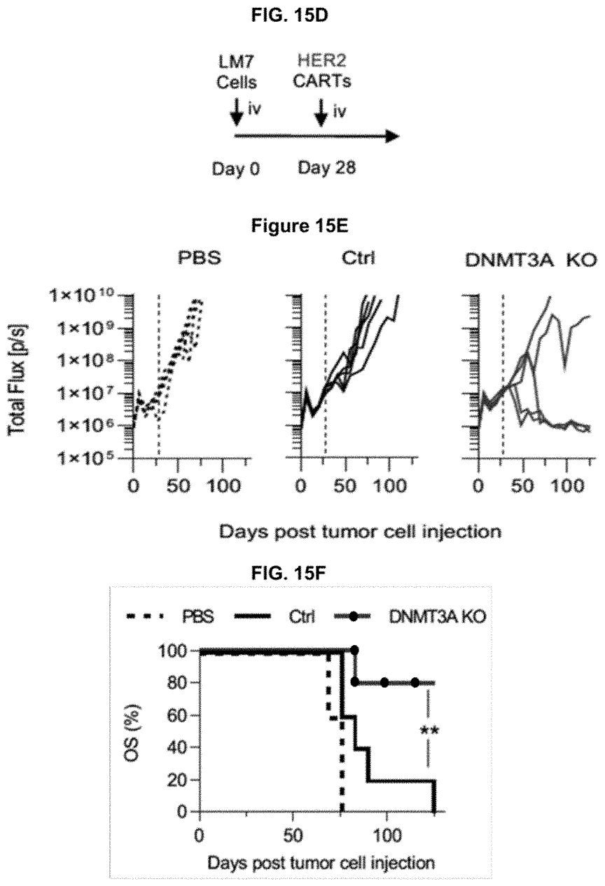

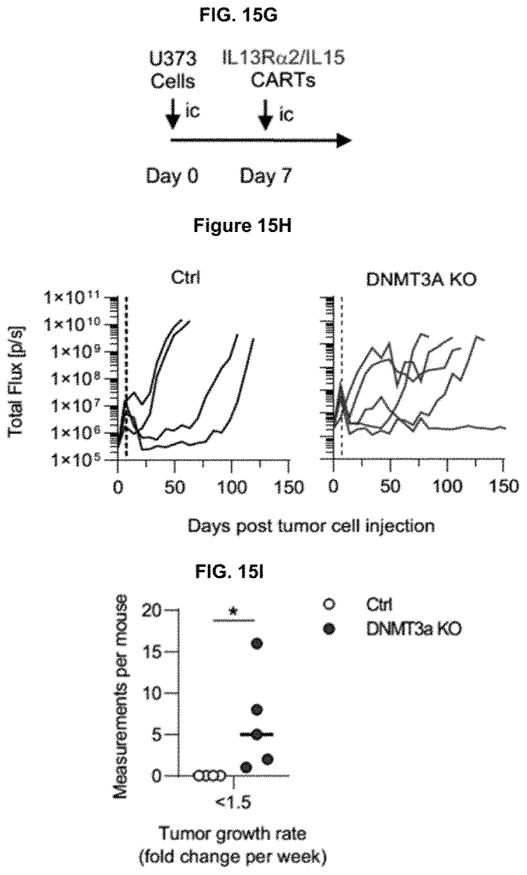

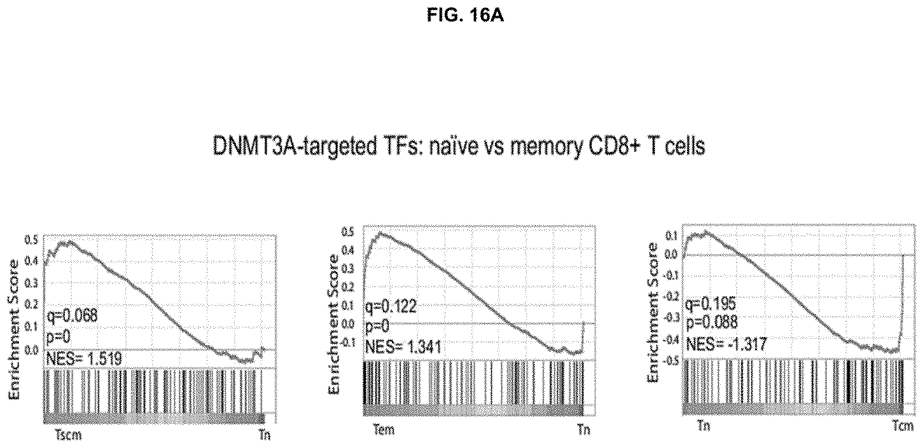

The impact of DNMT3A knockout on the antitumor activity of T cells expressing EphA2-CAR.CD28.ζ HER2-CAR.ζ, or IL13Rα2-CAR.CD28.ζ was evaluated in vivo. CAR expression and DNMT3A knockout for each CAR T cell population used in the in vivo solid tumor studies were confirmed with flow cytometry and Western Blot, respectively. Since significant differences was demonstrated between Ctrl.k/o and DNMT3A.k/o CAR T cells after chronic antigen exposure in vitro, the antitumor activity of antigen-exposed Ctrl.k/o and DNMT3A.k/o EphA2-CAR T cells was compared in the first animal model. Both EphA2-CAR T cell populations were stimulated with recombinant EphA2 protein for 48 hours before injection into LM7.GFPffLuc-bearing NSG mice (3×10⁵ T cells per mouse). While Ctrl.k/o and DNMT3A.k/o EphA2-CAR T cells both initially had significant antitumor activity, mice treated with Ctrl.k/o CAR T cells eventually experienced tumor relapse, whereas those treated with DNMT3A.k/o CAR T cells maintained tumor control, resulting in a significant survival advantage (FIGS. 15A-15C). As antigen-exposed DNMT3A.k/o CAR T cells have improved antitumor activity, DNMT3A.k/o HER2-CAR.ζ T cells, which had not been antigen pre-exposed, were evaluated next in vivo. LM7.GFPffLuc cells were injected intravenously into NSG mice to establish lung metastatic disease, and 28 days later mice received a single dose of 1×10⁶ Ctrl.k/o or DNMT3A.k/o HER2-CAR.ζ T cells, a cell dose that has limited efficacy in this model (35). While tumors progressed in Ctrl.k/o CAR T cell treated mice, 3/5 mice treated with DNMT3A.k/o HER2-CAR.ζ T cells had a complete response, resulting in a significant survival advantage (FIGS. 15D-15F note one DNMT3A.k/o CAR T cell treated animal has stable disease). In the last model, it was examined if DNMT3A knockout would further enhance the antitumor activity of CAR T cells that had been additionally genetically modified to improve their antitumor activity. The experiment was performed similarly to previous glioma studies that demonstrated transgenic expression of IL-15 in IL13Rα2-CAR T cells (IL13Rα2-CAR/IL15) improves their antitumor activity (34). Briefly, U373.eGFP.ffLuc cells were injected into the brain of NSG mice. Seven days later, mice received one intratumoral injection of 2×10⁶ Ctrl.k/o or DNMT3A.k/o IL13Rα2-CAR/IL15 T cells, and tumor growth was followed by bioluminescence imaging (FIGS. 15G-15H). Mice treated with DNMT3A.k/o CAR T cells had a median survival advantage of 33 days in comparison to mice treated with Ctrl.k/o CAR T cells (DNMT3A.k/o: 117 days; Ctrl.k/o: 84 days). Tumors in mice treated with DNMT3A.k/o CAR T cells grew significantly slower after initial progression (FIG. 15I). In addition, histological examination of tumors demonstrated a greater T cell infiltrate in tumors treated with DNMT3A.k/o CAR T cells in comparison Ctrl.k/o CAR T cells. Lastly, among tested tumor antigens IL13Rα2, HER2, and EphA2, one antigen (IL13Rα2) loss variant was observed post DNMT3A.k/o CAR T cell therapy. Despite this limitation, the presented data demonstrates that DNMT3A.k/o CAR T cells have improved antitumor activity compared to Ctrl.k/o CAR T cells in all three evaluated in vivo models regardless of targeted tumor antigen.

### Example 14. DNMT3A Knockout CAR T Cells Retain a Stem-Like Epigenetic Program During Prolonged Stimulation

The DNMT3A knockout enhances CAR T cell expansion and effector function during prolonged antigen exposure as demonstrated in the Examples above. In this Example, the cell intrinsic properties responsible for these differences were characterized. Given that DNMT3A.k/o and Ctrl.k/o CAR T cells were not readily distinguishable by standard flow cytometric analysis (assessment of CD45RA and CCR7 defined naïve and memory subsets did not show distinguishable difference between pre-stimulation and post-stimulation), changes in the epigenome of Ctrl.k/o and DNMT3A.k/o CAR T cells were evaluated by whole genome bisulfite sequencing (WGBS) after the 4^{th} tumor cell stimulation (FIG. 10C), a time point at which sufficient numbers of Ctrl.k/o CAR T cells are still available for analysis. More than 1,600 differentially methylated regions (DMRs) were identified among Ctrl.k/o and DNMT3A.k/o CAR T cells. These DMRs were predominantly localized to gene bodies (Exemplary DMRs include those in genes encoding transcription factors such as LEF1, TCF7, EOMES, ID2, DNMT3A, STAT5A, and BACH2, and homing loci such as ITGAE, CCR7, and SELL). Among the DNMT3A-mediated DMRs, a striking enrichment of transcription factors (TCF7, LEF1, BACH2) was observed that have been previously reported to be associated with terminal T cell differentiation (37-39). Notably, their promoter regions became fully methylated in contrast to DNMT3A.k/o CAR T cells (see promoter regions of DNMT3A and TCF7 in FIG. 10B). Additionally, the loci of several homing molecules, including ITGAE, CCR7, and SELL, also remained unmethylated in DNMT3A.k/o CAR T cells. Thus, the epigenetic programming of DNMT3A.k/o CAR T cells resembles the epigenetic signature of naïve or stem-cell like memory T cells, providing a direct mechanistic link to the preserved proliferative capacity and effector function observed in the DNMT3A.k/o CAR T cells.

To further explore the genes targeted by DNMT3A, a gene ontology (GO) analysis was performed. Strikingly, a significant number of transcriptional regulator loci were targeted for DNMT3A-mediated de novo methylation. Further characterization of the biological pathways affected by DNMT3A-regulated genes revealed statistically enriched pathways (p<0.001) associated with T cell effector differentiation, including T-cell receptor signaling, leukocyte migration, cytokine signaling, and apoptotic regulation. Ingenuity pathway analysis (IPA) of the DNMT3A-regulated genes indicated they were coupled to T-cell receptor signaling and Th1 and Th2 activation. In summary, these data highlight the critical role of DNMT3A in regulating human CAR T cell differentiation.

Given the striking enrichment of transcription factors targeted by DNMT3A, the developmental multipotential of DNMT3A.k/o CAR T cells was further interrogated. A machine learning approach was recently used to analyze the extensive collection of human CD8+ T cell WGBS datasets to develop a novel methylation-based index that predicts the multipotent capacity of human T cells. Briefly, whole-genome DNA methylation profiles of naïve and HIV-specific CD8+ T cells (virus-specific T cells obtained from chronically infected individuals) were used as training datasets for a machine learning analysis to define a set of CpG sites that establish a range of T cell differentiation states. This CpG site methylation index was then validated using whole-genome DNA methylation from a range of T cell subsets with well-defined differentiation capacities. This test dataset included whole-genome DNA methylation profiles of central-memory (Tcm), effector-memory (Tem), stem-cell memory (Tscm), and auto-reactive beta cell-specific CD8 T cells from patients with type 1 diabetes. Using this multipotency index, it was found the DNMT3A.k/o CAR T cells are predicted to have greater developmental plasticity than the stem-like subset of human memory T cells (Tscm) (FIG. 10A). In contrast, the control knockout CAR T cells had a predicted multipotency similar to short-lived Tems (FIG. 10A). Thus, consistent with the results from the functional characterization of DNMT3A.k/o CAR T cells, the novel methylation-based multipotency index indicates that DNMT3A programming is critical for limiting the developmental plasticity of chronically stimulated CAR T cells.

The progressive decline in a CAR T cell's proliferative capacity and effector function experienced during chronic stimulation has been ascribed to the process of T cell exhaustion (40). While the functional impairments of chronically stimulated CAR T cells are indeed hallmarks of exhaustion, a molecular signature of this state remains to be fully defined. Importantly, Dnmt3a-mediated de novo DNA methylation programming was previously demonstrated to be a critical regulator of T cell exhaustion in the prototypical murine model of chronic viral infection (40). With the understanding of the mechanisms involved in T cell exhaustion derived from a well-established model system, the overlap was explored between epigenetic programs linked to CAR T cell functional impairment with epigenetic programs that promote bona fide T cell exhaustion. To accomplish this, the published murine exhaustion DMR list was cross-referenced with the newly identified human CAR T cell terminal differentiation DMRs. It was found that among the 1,044 DNMT3A-targeted genes in chronically stimulated human CAR T cells, 337 genes were also selectively targeted by Dnmt3a during murine T cell exhaustion. This enrichment for epigenetically regulated exhaustion-associated genes in the human CAR T cell dataset indicates that epigenetic imprinting of exhaustion programs is a conserved biological mechanism limiting T cell effector responses. Taken together, these data demonstrate that DNMT3A promotes epigenetic silencing of genes that regulate the differentiation potential of human T cells and regulates CAR T cell exhaustion.

### Example 15. DNMT3A Deletion Promotes the Selective Survival of Developmentally Plastic CD8+ T Cells During Chronic Stimulation

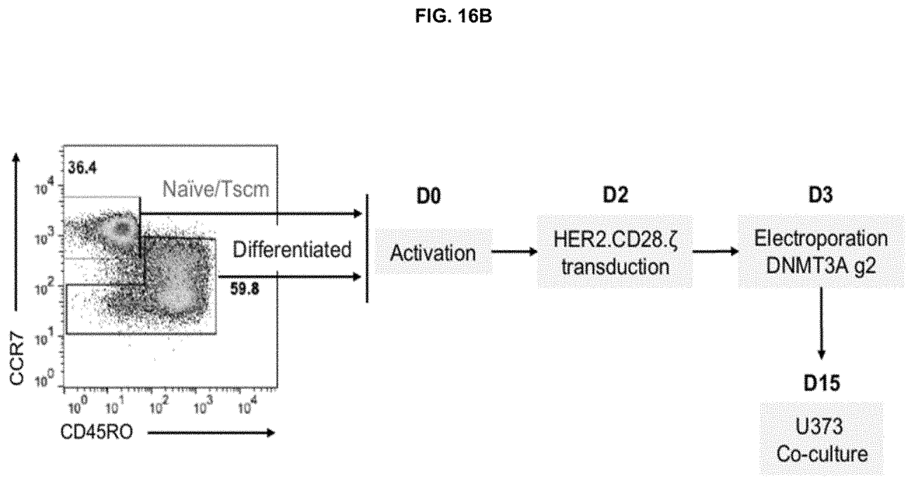

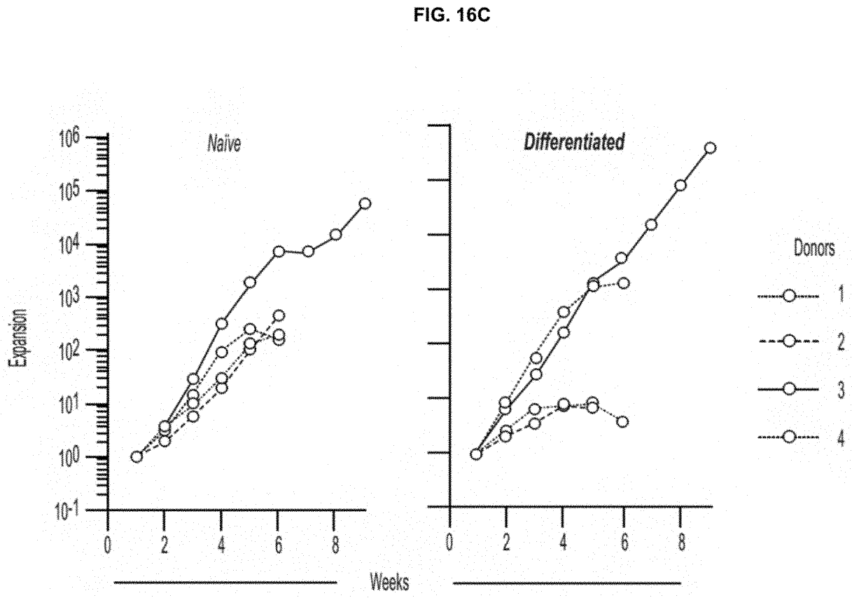

The data presented above indicate that DNMT3A deletion enriches for CAR T cells with a broad range of developmental potential under conditions of repeated tumor exposure. However it remains unclear if knocking out DNMT3A improves the developmental potential of the total pool of T cells, or if it preserves and/or selectively enriches for T cells that inherently possess a heightened differentiation capacity. To gain insight into this question, a gene set enrichment analysis (GSEA) was performed on DNMT3A-targeted genes and existing gene expression profiles from resting human naïve and memory CD8+ T cell subsets (FIG. 16A). Interestingly, DNMT3A-associated gene enrichment scores were most similar to memory T cell subsets in comparison to naïve T cells. Next it was determined if DNMT3A deletion results in selective survival of a population of T cells by establishing DNMT3A.k/o CAR T cells from purified naïve or memory CD8+ T cell subsets. These cells were generated by first sorting naïve/Tscm (CD45RA+, CCR7+) and memory (CD45RA−CCR7−; CD45RA−CCR7+; CD45RA+CCR7−) CD8+ T cells (FIG. 16B), followed by CAR transduction and DNMT3A knockout. Gene-edited CAR T cells were expanded for 10 days before performing the standard repeat coculture assay (FIG. 1B). For all 4 donors, DNMT3A.k/o CAR T cells derived from the naïve/Tscm T cell subset showed robust expansion; however, expansion was variable from DNMT3A.k/o CAR T cells derived from memory T cells (FIG. 16C). Thus, DNMT3A knockout consistently enhances effector function in CAR T cells derived from the naïve/Tscm T cell subset, whereas the benefit is donor dependent in CAR T cells derived from the memory compartment.

Collectively the Examples presented above demonstrate that genetic disruption of DNMT3A in CAR T cells prevents methylation of several key genes regulating human T cell differentiation. The resulting stem-like CAR T cells maintain their antigen-dependent proliferative capacity and effector functions during prolonged stimulation both in vitro and in vivo where they exhibit superior tumor control. The results here demonstrate that deletion of DNMT3A in CAR T cells may be an effective approach for maintaining the developmental potential of CAR T cells. Furthermore, preservation of the DNMT3A knockout CAR T cell proliferative capacity appears to occur without directly resulting in uncontrolled cellular division as seen in malignancies. Thus, these data provide further rationale for epigenetic-based approaches to extend the survival and effector response of CAR T cells.

The retained proliferative capacity of the DNMT3A knockout CAR T cells disclosed here is completely dependent upon the presence of antigen. Further, deletion of DNMT3A after the thymic development of naïve T cells does not appear to result in malignant transformation.

Rather than resulting in uncontrolled cellular division, the whole-genome DNA methylation profiles suggest that DNMT3A knockout CAR T cell's retained survival and effector capacity is coupled to maintenance of pre-existing epigenetically permissive programs at stemness-associated loci, including TCF7 and LEF1. The findings described herein demonstrate the importance of preserving a stem-like differentiation state of human T cells in establishing effective cellular therapies. While the results focus on CAR T cells, this approach can be broadly applied to the field of T cell therapy. Given that DNMT3A deletion enables first generation CARs (CARs lacking a costimulatory endodomain and more closely resembling conventional TCR signaling) to persist and kill tumors during chronic stimulation, this epigenetic process likely serves as a central mechanism for reinforcing the gene expression programs that are coupled to TCR signal duration and strength.

The results presented herein provide evidence that DNA methylation serves as a cell-intrinsic epigenetic modification that not only regulates T cell differentiation but also promotes CAR T cell exhaustion. The results show that CAR T cell exhaustion is coupled to de novo DNA methylation of genes that regulate T cell multipotency.

To provide a molecular definition of CAR T cell exhaustion, the analyses using the LCMV model system were applied which provided the technical and conceptual framework for this biological process (49, 50). Comparing WGBS nucleotide-resolution methylation profiles of control versus DNMT3A knockout CAR T cells, a genome-wide atlas of genes targeted by DNMT3A was established. A large portion of these DNMT3A-targeted genes are transcriptional regulators involved in limiting the “stemness” of immune cells. The striking overlap among the genes targeted by DNMT3A in mouse and human models of T cell exhaustion suggests that this epigenetic process may serve as a universal mechanism to restrict mammalian T cell fate potential.

Although exhaustion represents one of several barriers that may limit effective CAR T cell therapy, the results above further support the concept that epigenetic manipulation of CAR T cells directly impacts cellular function. Results from this study further highlight the importance of DNA methylation in regulating human T cell survival, and how approaches that manipulate this process can have an impact on the efficacy of T cell based therapies.

### REFERENCES

1. Park J H, Geyer M B, Brentjens R J. CD19-targeted CAR T-cell therapeutics for hematologic malignancies: interpreting clinical outcomes to date. Blood. 2016; 127(26):3312-20. doi: 10.1182/blood-2016-02-629063. PubMed PMID: 27207800; PMCID: 4929923.
2. Scarfo I, Maus M V. Current approaches to increase CAR T cell potency in solid tumors: targeting the tumor microenvironment. J Immunother Cancer. 2017; 5:28. doi: 10.1186/s40425-017-0230-9. PubMed PMID: 28331617; PMCID: PMC5359946.
3. Leibman R S, Richardson M W, Ellebrecht C T, Maldini C R, Glover J A, Secreto A J, Kulikovskaya I, Lacey S F, Akkina S R, Yi Y J, Shaheen F, Wang J B, Dufendach K A, Holmes M C, Collman R G, Payne A S, Riley J L. Supraphysiologic control over HIV-1 replication mediated by CD8 T cells expressing a re-engineered CD4-based chimeric antigen receptor. Plos Pathogens. 2017; 13(10). doi: ARTN e1006613 10.1371/journal.ppat.1006613. PubMed PMID: WOS:000414163300006.
4. Deeks S G, Wagner B, Anton P A, Mitsuyasu R T, Scadden D T, Haung C, Macken C, Richman D D, Christopherson C, June C H, Lazar R, Broad D F, Jalali S, Hege K M. A phase II randomized study of HIV-specific T-cell gene therapy in subjects with undetectable plasma viremia on combination antiretroviral therapy. Molecular Therapy. 2002; 5(6):788-97. doi: 10.1006/mthe.2002.0611. PubMed PMID: WOS:000176082700020.
5. Lam S, Bollard C. T-cell therapies for HIV. Immunotherapy-Uk. 2013; 5(4):407-14. doi: 10.2217/Imt.13.23. PubMed PMID: WOS:000317055200013.
6. Patel S, Jones R B, Nixon D F, Bollard C M. T-cell therapies for HIV: Preclinical successes and current clinical strategies. Cytotherapy. 2016; 18(8):931-42. Epub 2016/06/07. doi: 10.1016/j.jcyt.2016.04.007. PubMed PMID: 27265874; PMCID: PMC4935558.
7. Long A H, Haso W M, Shern J F, Wanhainen K M, Murgai M, Ingaramo M, Smith J P, Walker A J, Kohler M E, Venkateshwara V R, Kaplan R N, Patterson G H, Fry T J, Orentas R J, Mackall C L. 4-1BB costimulation ameliorates T cell exhaustion induced by tonic signaling of chimeric antigen receptors. Nature medicine. 2015; 21(6):581-90. doi: 10.1038/nm.3838. PubMed PMID: 25939063; PMCID: 4458184.
8. Ghosh A, Smith M, James S E, Davila M L, Velardi E, Argyropoulos K V, Gunset G, Perna F, Kreines F M, Levy E R, Lieberman S, Jay H V, Tuckett A Z, Zakrzewski J L, Tan L, Young L F, Takvorian K, Dudakov J A, Jenq R R, Hanash A M, Motta A C, Murphy G F, Liu C, Schietinger A, Sadelain M, van den Brink M R. Donor CD19 CAR T cells exert potent graft-versus-lymphoma activity with diminished graft-versus-host activity. Nat Med. 2017; 23(2):242-9. doi: 10.1038/nm.4258. PubMed PMID: 28067900.
9. Yang Y, Kohler M E, Chien C D, Sauter C T, Jacoby E, Yan C, Hu Y, Wanhainen K, Qin H, Fry T J. TCR engagement negatively affects CD8 but not CD4 CAR T cell expansion and leukemic clearance. Sci Transl Med. 2017; 9(417). doi: 10.1126/scitranslmed.aag1209. PubMed PMID: 29167392.
10. Zheng W, O'Hear C E, Alli R, Basham J H, Abdelsamed H A, Palmer L E, Jones L L, Youngblood B, Geiger T L. PI3K orchestration of the in vivo persistence of chimeric antigen receptor-modified T cells. Leukemia. 2018; 32(5):1157-67. doi: 10.1038/s41375-017-0008-6. PubMed PMID: 29479065; PMCID: PMC5943191.
11. Maude S L, Laetsch T W, Buechner J, Rives S, Boyer M, Bittencourt H, Bader P, Verneris M R, Stefanski H E, Myers G D, Qayed M, De Moerloose B, Hiramatsu H, Schlis K, Davis K L, Martin P L, Nemecek E R, Yanik G A, Peters C, Baruchel A, Boissel N, Mechinaud F, Balduzzi A, Krueger J, June C H, Levine B L, Wood P, Taran T, Leung M, Mueller K T, Zhang Y, Sen K, Lebwohl D, Pulsipher M A, Grupp S A. Tisagenlecleucel in Children and Young Adults with B-Cell Lymphoblastic Leukemia. The New England journal of medicine. 2018; 378(5):439-48. doi: 10.1056/NEJMoa1709866. PubMed PMID: 29385370.
12. Fraietta J A, Lacey S F, Orlando E J, Pruteanu-Malinici I, Gohil M, Lundh S, Boesteanu A C, Wang Y, O'Connor R S, Hwang W T, Pequignot E, Ambrose D E, Zhang C, Wilcox N, Bedoya F, Dorfmeier C, Chen F, Tian L, Parakandi H, Gupta M, Young R M, Johnson F B, Kulikovskaya I, Liu L, Xu J, Kassim S H, Davis M M, Levine B L, Frey N V, Siegel D L, Huang A C, Wherry E J, Bitter H, Brogdon J L, Porter D L, June C H, Melenhorst J J. Determinants of response and resistance to CD19 chimeric antigen receptor (CAR) T cell therapy of chronic lymphocytic leukemia. Nat Med. 2018; 24(5):563-71. Epub 2018/05/02. doi: 10.1038/s41591-018-0010-1. PubMed PMID: 29713085; PMCID: PMC6117613.
13. Ahmed N, Brawley V S, Hegde M, Robertson C, Ghazi A, Gerken C, Liu E, Dakhova O, Ashoori A, Corder A, Gray T, Wu M F, Liu H, Hicks J, Rainusso N, Dotti G, Mei Z, Grilley B, Gee A, Rooney C M, Brenner M K, Heslop H E, Wels W S, Wang L L, Anderson P, Gottschalk S. Human Epidermal Growth Factor Receptor 2 (HER2)-Specific Chimeric Antigen Receptor-Modified T Cells for the Immunotherapy of HER2-Positive Sarcoma. Journal of clinical oncology: official journal of the American Society of Clinical Oncology. 2015; 33(15):1688-96. doi: 10.1200/JCO.2014.58.0225. PubMed PMID: 25800760; PMCID: 4429176.
14. Beatty G L, O'Hara M R, Lacey S F, Torigian D A, Nazimuddin F, Chen F, Kulikovskaya I M, Soulen M C, McGarvey M, Nelson A M, Gladney W L, Levine B L, Melenhorst J J, Plesa G, June C H. Activity of Mesothelin-specific Chimeric Antigen Receptor T cells Against Pancreatic Carcinoma Metastases in a Phase 1 Trial. Gastroenterology. 2018. doi: 10.1053/j.gastro.2018.03.029. PubMed PMID: 29567081.
15. Thistlethwaite F C, Gilham D E, Guest R D, Rothwell D G, Pillai M, Burt D J, Byatte A J, Kirillova N, Valle J W, Sharma S K, Chester K A, Westwood N B, Halford SER, Nabarro S, Wan S, Austin E, Hawkins R E. The clinical efficacy of first-generation carcinoembryonic antigen (CEACAM5)-specific CAR T cells is limited by poor persistence and transient pre-conditioning-dependent respiratory toxicity. Cancer immunology, immunotherapy: CII. 2017; 66(11):1425-36. Epub 2017/07/01. doi: 10.1007/s00262-017-2034-7. PubMed PMID: 28660319; PMCID: PMC5645435.
16. Heczey A, Louis C U, Savoldo B, Dakhova O, Durett A, Grilley B, Liu H, Wu M F, Mei Z, Gee A, Mehta B, Zhang H, Mahmood N, Tashiro H, Heslop H E, Dotti G, Rooney C M, Brenner M K. CAR T Cells Administered in Combination with Lymphodepletion and PD-1 Inhibition to Patients with Neuroblastoma. Molecular therapy: the journal of the American Society of Gene Therapy. 2017; 25(9):2214-24. doi: 10.1016/j.ymthe.2017.05.012. PubMed PMID: 28602436; PMCID: PMC5589058.
17. O'Rourke D M, Nasrallah M P, Desai A, Melenhorst J J, Mansfield K, Morrissette JJD, Martinez-Lage M, Brem S, Maloney E, Shen A, Isaacs R, Mohan S, Plesa G, Lacey S F, Navenot J M, Zheng Z, Levine B L, Okada H, June C H, Brogdon J L, Maus M V. A single dose of peripherally infused EGFRvIII-directed CAR T cells mediates antigen loss and induces adaptive resistance in patients with recurrent glioblastoma. Science translational medicine. 2017; 9(399). doi: 10.1126/scitranslmed.aaa0984. PubMed PMID: 28724573.
18. Brown C E, Alizadeh D, Starr R, Weng L, Wagner J R, Naranjo A, Ostberg J R, Blanchard M S, Kilpatrick J, Simpson J, Kurien A, Priceman S J, Wang X, Harshbarger T L, D′Apuzzo M, Ressler J A, Jensen M C, Barish M E, Chen M, Portnow J, Forman S J, Badie B. Regression of Glioblastoma after Chimeric Antigen Receptor T-Cell Therapy. The New England journal of medicine. 2016; 375(26):2561-9. doi: 10.1056/NEJMoa1610497. PubMed PMID: 28029927.
19. Maude S L, Frey N, Shaw P A, Aplenc R, Barrett D M, Bunin N J, Chew A, Gonzalez V E, Zheng Z, Lacey S F, Mahnke Y D, Melenhorst J J, Rheingold S R, Shen A, Teachey D T, Levine B L, June C H, Porter D L, Grupp S A. Chimeric antigen receptor T cells for sustained remissions in leukemia. NEnglJMed. 2014; 371(16):1507-17. doi: 10.1056/NEJMoa1407222 [doi].
20. Pauken K E, Sammons M A, Odorizzi P M, Manne S, Godec J, Khan O, Drake A M, Chen Z, Sen D R, Kurachi M, Barnitz R A, Bartman C, Bengsch B, Huang A C, Schenkel J M, Vahedi G, Haining W N, Berger S L, Wherry E J. Epigenetic stability of exhausted T cells limits durability of reinvigoration by PD-1 blockade. Science. 2016; 354(6316):1160-5. Epub 2016/10/30. doi: 10.1126/science.aaf2807. PubMed PMID: 27789795.
21. Sen D R, Kaminski J, Barnitz R A, Kurachi M, Gerdemann U, Yates K B, Tsao H W, Godec J, LaFleur M W, Brown F D, Tonnerre P, Chung R T, Tully D C, Allen T M, Frahm N, Lauer G M, Wherry E J, Yosef N, Haining W N. The epigenetic landscape of T cell exhaustion. Science. 2016; 354(6316):1165-9. doi: 10.1126/science.aae0491. PubMed PMID: 27789799.
22. Ghoneim H E, Fan Y, Moustaki A, Abdelsamed H A, Dash P, Dogra P, Carter R, Awad W, Neale G, Thomas P G, Youngblood B. De Novo Epigenetic Programs Inhibit PD-1 Blockade-Mediated T Cell Rejuvenation. Cell. 2017; 170(1):142-57 e19. doi: 10.1016/j.cell.2017.06.007. PubMed PMID: 28648661; PMCID: PMC5568784.
23. Youngblood B, Noto A, Porichis F, Akondy R S, Ndhlovu Z M, Austin J W, Bordi R, Procopio F A, Miura T, Allen T M, Sidney J, Sette A, Walker B D, Ahmed R, Boss J M, Sekaly R P, Kaufmann D E. Cutting edge: Prolonged exposure to HIV reinforces a poised epigenetic program for PD-1 expression in virus-specific CD8 T cells. J Immunol. 2013; 191(2):540-4. Epub 2013/06/19. doi: 10.4049/jimmunol.1203161. PubMed PMID: 23772031; PMCID: 3702641.
24. Youngblood B, Oestreich K J, Ha S J, Duraiswamy J, Akondy R S, West E E, Wei Z, Lu P, Austin J W, Riley J L, Boss J M, Ahmed R. Chronic virus infection enforces demethylation of the locus that encodes PD-1 in antigen-specific CD8(+) T cells. Immunity. 2011; 35(3):400-12. Epub 2011/09/29. doi: 10.1016/j.immuni.2011.06.015. PubMed PMID: 21943489; PMCID: 3183460.
25. Nakayama-Hosoya K, Ishida T, Youngblood B, Nakamura H, Hosoya N, Koga M, Koibuchi T, Iwamoto A, Kawana-Tachikawa A. Epigenetic Repression of Interleukin 2 Expression in Senescent CD4+ T Cells During Chronic HIV Type 1 Infection. J Infect Dis. 2014. doi: 10.1093/infdis/jiu376. PubMed PMID: 25001463.
26. Scharer C D, Barwick B G, Youngblood B A, Ahmed R, Boss J M. Global DNA Methylation Remodeling Accompanies CD8 T Cell Effector Function. Journal of immunology. 2013. doi: 10.4049/jimmunol.1301395. PubMed PMID: 23956425.
27. West E E, Youngblood B, Tan W G, Jin H T, Araki K, Alexe G, Konieczny B T, Calpe S, Freeman G J, Terhorst C, Haining W N, Ahmed R. Tight Regulation of Memory CD8(+) T Cells Limits Their Effectiveness during Sustained High Viral Load. Immunity. 2011; 35(2):285-98. PubMed PMID: 21856186.
28. Youngblood B, Oestreich K J, Ha S J, Duraiswamy J, Akondy R S, West E E, Wei Z, Lu P, Austin J W, Riley J L, Boss J M, Ahmed R. Chronic virus infection enforces demethylation of the locus that encodes PD-1 in antigen-specific CD8+ T cells Immunity. 2011; 35(3):13.
29. Youngblood B, Wherry E J, Ahmed R. Acquired transcriptional programming in functional and exhausted virus-specific CD8 T cells. Curr Opin HIV AIDS. 2012; 7(1):50-7. Epub 2011/12/03. doi: 10.1097/COH.0b013e32834ddcf2. PubMed PMID: 22134341; PMCID: 3306172.
30. Abdelsamed H A, Moustaki A, Fan Y P, Dogra P, Ghoneim H E, Zebley C C, Triplett B M, Sekaly R P, Youngblood B. Human memory CD8 T cell effector potential is epigenetically preserved during in vivo homeostasis. Journal of Experimental Medicine. 2017; 214(6):1593-606. doi: 10.1084/jem.20161760. PubMed PMID: WOS:000402863300005.
31. Ahn E, Youngblood B, Lee J, Lee J, Sarkar S, Ahmed R. Demethylation of the PD-1 Promoter Is Imprinted during the Effector Phase of CD8 T Cell Exhaustion. J Virol. 2016; 90(19):8934-46. doi: 10.1128/JVI.00798-16. PubMed PMID: 27466420; PMCID: 5021389.
32. Blackburn S D, Shin H, Freeman G J, Wherry E J. Selective expansion of a subset of exhausted CD8 T cells by alphaPD-L1 blockade. Proc Natl Acad Sci USA. 2008; 105(39):15016-21. Epub 2008/09/24. doi: 10.1073/pnas.0801497105. PubMed PMID: 18809920; PMCID: PMC2567485.
33. Liao J, Karnik R, Gu H, Ziller M J, Clement K, Tsankov A M, Akopian V, Gifford C A, Donaghey J, Galonska C, Pop R, Reyon D, Tsai S Q, Mallard W, Joung J K, Rinn J L, Gnirke A, Meissner A. Targeted disruption of DNMT1, DNMT3A and DNMT3B in human embryonic stem cells. Nat Genet. 2015; 47(5):469-78. Epub 2015/03/31. doi: 10.1038/ng.3258. PubMed PMID: 25822089; PMCID: PMC4414868.
34. Krenciute G, Prinzing B L, Yi Z, Wu M F, Liu H, Dotti G, Balyasnikova I V, Gottschalk S. Transgenic Expression of IL15 Improves Antiglioma Activity of IL13Ralpha2-CAR T Cells but Results in Antigen Loss Variants. Cancer immunology research. 2017; 5(7):571-81. doi: 10.1158/2326-6066.CIR-16-0376. PubMed PMID: 28550091; PMCID: PMC5746871.
35. Mata M, Gerken C, Nguyen P, Krenciute G, Spencer D M, Gottschalk S. Inducible Activation of MyD88 and CD40 in CAR T Cells Results in Controllable and Potent Antitumor Activity in Preclinical Solid Tumor Models. Cancer discovery. 2017; 7(11):1306-19. doi: 10.1158/2159-8290.CD-17-0263. PubMed PMID: 28801306; PMCID: PMC5780189.
36. Fujii S, Shimizu K, Shimizu T, Lotze M T. Interleukin-10 promotes the maintenance of antitumor CD8(+) T-cell effector function in situ. Blood. 2001; 98(7):2143-51. Epub 2001/09/25. doi: 10.1182/blood.v98.7.2143. PubMed PMID: 11568001.
37. Roychoudhuri R, Clever D, Li P, Wakabayashi Y, Quinn K M, Klebanoff C A, Ji Y, Sukumar M, Eil R L, Yu Z, Spolski R, Palmer D C, Pan J H, Patel S J, Macallan D C, Fabozzi G, Shih H Y, Kanno Y, Muto A, Zhu J, Gattinoni L, O'Shea J J, Okkenhaug K, Igarashi K, Leonard W J, Restifo N P. BACH2 regulates CD8(+) T cell differentiation by controlling access of AP-1 factors to enhancers. Nat Immunol. 2016; 17(7):851-60. Epub 2016/05/10. doi: 10.1038/ni.3441. PubMed PMID: 27158840; PMCID: PMC4918801.
38. Kaech S M, Cui W. Transcriptional control of effector and memory CD8+ T cell differentiation. Nat Rev Immunol. 2012; 12(11):749-61. Epub 2012/10/20. doi: 10.1038/nri3307. PubMed PMID: 23080391; PMCID: PMC4137483.
39. Chen Y, Zander R, Khatun A, Schauder D M, Cui W. Transcriptional and Epigenetic Regulation of Effector and Memory CD8 T Cell Differentiation. Front Immunol. 2018; 9:2826. Epub 2018/12/26. doi: 10.3389/fimmu.2018.02826. PubMed PMID: 30581433; PMCID: PMC6292868.
40. Schultz L, Mackall C. Driving CAR T cell translation forward. Science translational medicine. 2019; 11(481). Epub 2019/03/01. doi: 10.1126/scitranslmed.aaw2127. PubMed PMID: 30814337.
41. Feucht J, Sun J, Eyquem J, Ho Y J, Zhao Z, Leibold J, Dobrin A, Cabriolu A, Hamieh M, Sadelain M. Calibration of CAR activation potential directs alternative T cell fates and therapeutic potency. Nature medicine. 2019; 25(1):82-8. Epub 2018/12/19. doi: 10.1038/s41591-018-0290-5. PubMed PMID: 30559421; PMCID: PMC6532069.
42. Sommermeyer D, Hudecek M, Kosasih P L, Gogishvili T, Maloney D G, Turtle C J, Riddell S R. Chimeric antigen receptor-modified T cells derived from defined CD8+ and CD4+subsets confer superior antitumor reactivity in vivo. Leukemia. 2016; 30(2):492-500. doi: 10.1038/1eu.2015.247. PubMed PMID: 26369987; PMCID: PMC4746098.
43. Gattinoni L, Lugli E, Ji Y, Pos Z, Paulos C M, Quigley M F, Almeida J R, Gostick E, Yu Z, Carpenito C, Wang E, Douek D C, Price D A, June C H, Marincola F M, Roederer M, Restifo N P. A human memory T cell subset with stem cell-like properties. NatMed. 2011; 17(10):1290-7. doi: nm.2446 [pii]; 10.1038/nm.2446 [doi].
44. Ley T J, Ding L, Walter M J, McLellan M D, Lamprecht T, Larson D E, Kandoth C, Payton J E, Baty J, Welch J, Harris C C, Lichti C F, Townsend R R, Fulton R S, Dooling D J, Koboldt D C, Schmidt H, Zhang Q, Osborne J R, Lin L, O'Laughlin M, McMichael J F, Delehaunty K D, McGrath S D, Fulton L A, Magrini V J, Vickery T L, Hundal J, Cook L L, Conyers J J, Swift G W, Reed J P, Alldredge P A, Wylie T, Walker J, Kalicki J, Watson M A, Heath S, Shannon W D, Varghese N, Nagarajan R, Westervelt P, Tomasson M H, Link D C, Graubert T A, DiPersio J F, Mardis E R, Wilson R K. DNMT3A mutations in acute myeloid leukemia. The New England journal of medicine. 2010; 363(25):2424-33. Epub 2010/11/12. doi: 10.1056/NEJMoa1005143. PubMed PMID: 21067377; PMCID: PMC3201818.
45. Walter M J, Ding L, Shen D, Shao J, Grillot M, McLellan M, Fulton R, Schmidt H, Kalicki-Veizer J, O'Laughlin M, Kandoth C, Baty J, Westervelt P, DiPersio J F, Mardis E R, Wilson R K, Ley T J, Graubert T A. Recurrent DNMT3A mutations in patients with myelodysplastic syndromes. Leukemia. 2011; 25(7):1153-8. Epub 2011/03/19. doi: 10.1038/1eu.2011.44. PubMed PMID: 21415852; PMCID: PMC3202965.
46. Youngblood B, Hale J S, Kissick H T, Ahn E, Xu X, Wieland A, Araki K, West E E, Ghoneim H E, Fan Y, Dogra P, Davis C W, Konieczny B T, Antia R, Cheng X, Ahmed R. Effector CD8 T cells dedifferentiate into long-lived memory cells. Nature. 2017; 552(7685):404-9. doi: 10.1038/nature25144. PubMed PMID: 29236683; PMCID: PMC5965677.
47. Kramer A C, Kothari A, Wilson W C, Celik H, Nikitas J, Mallaney C, Ostrander E L, Eultgen E, Martens A, Valentine M C, Young A L, Druley T E, Figueroa M E, Zhang B, Challen G A. Dnmt3a regulates T-cell development and suppresses T-ALL transformation. Leukemia. 2017; 31(11):2479-90. Epub 2017/03/23. doi: 10.1038/1eu.2017.89. PubMed PMID: 28321121; PMCID: PMC5636646.
48. Sade-Feldman M, Yizhak K, Bjorgaard S L, Ray J P, de Boer C G, Jenkins R W, Lieb D J, Chen J H, Frederick D T, Barzily-Rokni M, Freeman S S, Reuben A, Hoover P J, Villani A C, Ivanova E, Portell A, Lizotte P H, Aref A R, Eliane J P, Hammond M R, Vitzthum H, Blackmon S M, Li B, Gopalakrishnan V, Reddy S M, Cooper Z A, Paweletz C P, Barbie D A, Stemmer-Rachamimov A, Flaherty K T, Wargo J A, Boland G M, Sullivan R J, Getz G, Hacohen N. Defining T Cell States Associated with Response to Checkpoint Immunotherapy in Melanoma. Cell. 2018; 175(4):998-1013 e20. Epub 2018/11/06. doi: 10.1016/j.ce11.2018.10.038. PubMed PMID: 30388456.
49. Moskophidis D, Lechner F, Pircher H, Zinkernagel R M. Virus persistence in acutely infected immunocompetent mice by exhaustion of antiviral cytotoxic effector T cells. Nature. 1993; 362(6422):758-61. Epub 1993/04/22. doi: 10.1038/362758a0. PubMed PMID: 8469287.
50. Zajac A J, Blattman J N, Murali-Krishna K, Sourdive D J, Suresh M, Altman J D, Ahmed R. Viral immune evasion due to persistence of activated T cells without effector function. J Exp Med. 1998; 188(12):2205-13. Epub 1998/12/22. doi: 10.1084/jem.188.12.2205. PubMed PMID: 9858507; PMCID: PMC2212420.
51. Lynn R C, Weber E W, Sotillo E, Gennert D, Xu P, Good Z, Anbunathan H, Lattin J, Jones R, Tieu V, Nagaraj a S, Granja J, de Bourcy CFA, Majzner R, Satpathy A T, Quake S R, Monje M, Chang H Y, Mackall C L. c-Jun overexpression in CAR T cells induces exhaustion resistance. Nature. 2019; 576(7786):293-300. Epub 2019/12/06. doi: 10.1038/s41586-019-1805-z. PubMed PMID: 31802004.
52. Alfei F, Kanev K, Hofmann M, Wu M, Ghoneim H E, Roelli P, Utzschneider D T, von Hoesslin M, Cullen J G, Fan Y, Eisenberg V, Wohlleber D, Steiger K, Merkler D, Delorenzi M, Knolle P A, Cohen C J, Thimme R, Youngblood B, Zehn D. TOX reinforces the phenotype and longevity of exhausted T cells in chronic viral infection. Nature. 2019; 571(7764):265-9. Epub 2019/06/18. doi: 10.1038/s41586-019-1326-9. PubMed PMID: 31207605.
53. Khan O, Giles J R, McDonald S, Manne S, Ngiow S F, Patel K P, Werner M T, Huang A C, Alexander K A, Wu J E, Attanasio J, Yan P, George S M, Bengsch B, Staupe R P, Donahue G, Xu W, Amaravadi R K, Xu X, Karakousis G C, Mitchell T C, Schuchter L M, Kaye J, Berger S L, Wherry E J. TOX transcriptionally and epigenetically programs CD8(+) T cell exhaustion. Nature. 2019; 571(7764):211-8. Epub 2019/06/18. doi: 10.1038/s41586-019-1325-x. PubMed PMID: 31207603; PMCID: PMC6713202.
54. Scott A C, Dundar F, Zumbo P, Chandran S S, Klebanoff C A, Shakiba M, Trivedi P, Menocal L, Appleby H, Camara S, Zamarin D, Walther T, Snyder A, Femia M R, Comen E A, Wen H Y, Hellmann M D, Anandasabapathy N, Liu Y, Altorki N K, Lauer P, Levy O, Glickman M S, Kaye J, Betel D, Philip M, Schietinger A. TOX is a critical regulator of tumour-specific T cell differentiation. Nature. 2019; 571(7764):270-4. Epub 2019/06/18. doi: 10.1038/s41586-019-1324-y. PubMed PMID: 31207604.
55. Fraietta J A, Nobles C L, Sammons M A, Lundh S, Carty S A, Reich T J, Cogdill A P, Morrissette J J D, DeNizio J E, Reddy S, Hwang Y, Gohil M, Kulikovskaya I, Nazimuddin F, Gupta M, Chen F, Everett J K, Alexander K A, Lin-Shiao E, Gee M R, Liu X, Young R M, Ambrose D, Wang Y, Xu J, Jordan M S, Marcucci K T, Levine B L, Garcia K C, Zhao Y, Kalos M, Porter D L, Kohli R M, Lacey S F, Berger S L, Bushman F D, June C H, Melenhorst J J. Disruption of TET2 promotes the therapeutic efficacy of CD19-targeted T cells. Nature. 2018; 558(7709):307-12. doi: 10.1038/s41586-018-0178-z. PubMed PMID: 29849141.
56. Chow K K, Naik S, Kakarla S, Brawley V S, Shaffer D R, Yi Z, Rainusso N, Wu M F, Liu H, Kew Y, Grossman R G, Powell S, Lee D, Ahmed N, Gottschalk S. T Cells Redirected to EphA2 for the Immunotherapy of Glioblastoma. Mol Ther. 2013; 21(3):629-37. doi: mt2012210 [pii]; 10.1038/mt.2012.210 [doi].
57. Krenciute G, Krebs S, Torres D, Wu M F, Liu H, Dotti G, Li X N, Lesniak M S, Balyasnikova I V, Gottschalk S. Characterization and Functional Analysis of scFv-based Chimeric Antigen Receptors to Redirect T Cells to IL13 Ralpha2-positive Glioma. Molecular therapy: the journal of the American Society of Gene Therapy. 2016; 24(2):354-63. doi: 10.1038/mt.2015.199. PubMed PMID: 26514825; PMCID: PMC4817815.
58. Yi Z, Prinzing B L, Cao F, Gottschalk S, Krenciute G. Optimizing EphA2-CAR T Cells for the Adoptive Immunotherapy of Glioma. Mol Ther Methods Clin Dev. 2018; 9:70-80. doi: 10.1016/j.omtm.2018.01.009. PubMed PMID: 29552579; PMCID: PMC5852415.
59. Quintarelli C, Vera J F, Savoldo B, Giordano Attianese G M, Pule M, Foster A E, Heslop H E, Rooney C M, Brenner M K, Dotti G. Co-expression of cytokine and suicide genes to enhance the activity and safety of tumor-specific cytotoxic T lymphocytes. Blood. 2007; 110(8):2793-802.
60. Ahmed N, Ratnayake M, Savoldo B, Perlaky L, Dotti G, Wels W S, Bhattacharjee M B, Gilbertson R J, Shine H D, Weiss H L, Rooney C M, Heslop H E, Gottschalk S. Regression of experimental medulloblastoma following transfer of HER2-specific T cells. Cancer Res. 2007; 67(12):5957-64.
61. Ahmed N, Salsman V S, Yvon E, Louis C U, Perlaky L, Wels W S, Dishop M K, Kleinerman E E, Pule M, Rooney C M, Heslop H E, Gottschalk S. Immunotherapy for osteosarcoma: genetic modification of T cells overcomes low levels of tumor antigen expression. Mol Ther. 2009; 17(10):1779-87.
62. Li W, Guo L, Rathi P, Marinova E, Gao X, Wu M F, Liu H, Dotti G, Gottschalk S, Metelitsa L S, Heczey A. Redirecting T Cells to Glypican-3 with 4-1B B Zeta Chimeric Antigen Receptors Results in Th1 Polarization and Potent Antitumor Activity. Human gene therapy. 2017; 28(5):437-48. doi: 10.1089/hum.2016.025. PubMed PMID: 27530312; PMCID: PMC5444493.
63. Connelly J P, Pruett-Miller S M. CRIS.py: A Versatile and High-throughput Analysis Program for CRISPR-based Genome Editing. Scientific reports. 2019; 9(1):4194. Epub 2019/03/14. doi: 10.1038/s41598-019-40896-w. PubMed PMID: 30862905; PMCID: PMC6414496.
64. Abdelsamed H A, Moustaki A, Fan Y, Dogra P, Ghoneim H E, Zebley C C, Triplett B M, Sekaly R P, Youngblood B. Human memory CD8 T cell effector potential is epigenetically preserved during in vivo homeostasis. J Exp Med. 2017; 214(6):1593-606. doi: 10.1084/jem.20161760. PubMed PMID: 28490440; PMCID: PMC5461005.
65. Xi Y, Li W. BSMAP: whole genome bisulfite sequence MAPping program. BMC Bioinformatics. 2009; 10:232. Epub 2009/07/29. doi: 10.1186/1471-2105-10-232. PubMed PMID: 19635165; PMCID: PMC2724425.
66. Sun D, Xi Y, Rodriguez B, Park H J, Tong P, Meong M, Goodell M A, Li W. MOABS: model based analysis of bisulfite sequencing data. Genome biology. 2014; 15(2):R38. doi: 10.1186/gb-2014-15-2-r38. PubMed PMID: 24565500; PMCID: 4054608.
67. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: a knowledge-based approach for interpreting genome-wide expression profiles. Proceedings of the National Academy of Sciences of the United States of America. 2005; 102(43):15545-50. Epub 2005/10/04. doi: 10.1073/pnas.0506580102. PubMed PMID: 16199517; PMCID: PMC1239896.
68. Malta M D, Sokolov A, Gentles A J, Burzykowski T, Poisson L, Weinstein J N, Kaminska B, Huelsken J, Omberg L, Gevaert O, Colaprico A, Czerwinska P, Mazurek S, Mishra L, Heyn H, Krasnitz A, Godwin A K, Lazar A J, Cancer Genome Atlas Research N, Stuart J M, Hoadley K A, Laird P W, Noushmehr H, Wiznerowicz M. Machine Learning Identifies Stemness Features Associated with Oncogenic Dedifferentiation. Cell. 2018; 173(2):338-54 e15. Epub 2018/04/07. doi: 10.1016/j.cell.2018.03.034. PubMed PMID: 29625051; PMCID: PMC5902191.
69. Sokolov A, Carlin D E, Paull E O, Baertsch R, Stuart J M. Pathway-Based Genomics Prediction using Generalized Elastic Net. PLoS Comput Biol. 2016; 12(3):e1004790. Epub 2016/03/10.doi:10.1371/journal.pcbi.1004790. PubMed PMID: 26960204; PMCID: PMC4784899.
70. Robinson M D, McCarthy D J, Smyth G K. edgeR: a Bioconductor package for differential expression analysis of digital gene expression data. Bioinformatics. 2010; 26(1):139-40. Epub 2009/11/17. doi: 10.1093/bioinformatics/btp616. PubMed PMID: 19910308; PMCID: PMC2796818.
71. Ritchie M E, Phipson B, Wu D, Hu Y, Law C W, Shi W, Smyth G K. limma powers differential expression analyses for RNA-sequencing and microarray studies. Nucleic Acids Res. 2015; 43(7):e47. Epub 2015/01/22. doi: 10.1093/nar/gkv007. PubMed PMID: 25605792; PMCID: PMC4402510.
72. Wickham H. ggplot2: elegant graphics for data analysis: Springer; 2016.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A modified immune effector cell, wherein (i) DNA (cytosine-5)-methyltransferase 3A (DNMT3A)-mediated de novo DNA methylation of the cell genome is inhibited, and (ii) STAT5 signaling pathway is activated.

2. The modified immune effector cell of claim 1, wherein the enzymatic activity of the DNMT3A protein is inhibited in the cell.

3. The modified immune effector cell of claim 2, wherein the enzymatic activity of the DNMT3A protein is inhibited by exposing the cell to a DNMT3A active site inhibitor.

4. The modified immune effector cell of claim 2, wherein the DNMT3A gene is mutated in DNMT3A catalytic domain so that the enzymatic activity of the DNMT3A protein is inhibited.

5. The modified immune effector cell of claim 1, wherein the level of functional DNMT3A protein in the cell is decreased by 50% or more.

6. The modified immune effector cell of claim 1, wherein DNMT3A gene is deleted or defective so that no detectable functional DNMT3A protein is produced.

7. The modified immune effector cell of claim 1, wherein the immune effector cell is a T cell, natural killer (NK) cell, or a stem cell that is capable of differentiating into an immune cell.

8. The modified immune effector cell of claim 7, wherein the T cell is selected from a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg).

9. (canceled)

10. The modified immune effector cell of claim 7, wherein the stem cell is an induced pluripotent stem cell (iPSC).

11. (canceled)

12. The modified immune effector cell of claim 1, wherein the STAT5 signaling pathway is activated by a signaling molecule.

13. The modified immune effector cell of claim 12, wherein the signaling molecule is a common gamma chain cytokine.

14. The modified immune effector cell of claim 13, wherein the cytokine is IL-15, IL-7, IL-2, IL-4, IL-9, or IL-21.

15. The modified immune effector cell of claim 1, wherein the STAT5 signaling pathway is activated by modifying the immune effector cell to express a constitutively active cytokine receptor or a switch receptor.

16. The modified immune effector cell of claim 15, wherein the constitutively active cytokine receptor is a constitutively active IL7 receptor (C7R).

17. The modified immune effector cell of claim 15, wherein the switch receptor is an IL-4/IL-7 receptor or an IL-4/IL-2 receptor.

18. The modified immune effector cell of claim 1, wherein the cell further comprises at least one surface molecule capable of binding specifically to an antigen.

19. The modified immune effector cell of claim 18, wherein the antigen is selected from a tumor antigen, a viral antigen, a bacterial antigen, a fungal antigen, a parasite antigen, a prion antigen, and an antigen associated with an inflammation or an autoimmune disease.

20. (canceled)

21. The modified immune effector cell of claim 1, wherein the cell further comprises a chimeric antigen receptor (CAR), an antigen specific T-cell receptor, or a bispecific antibody.

22-57. (canceled)

58. A pharmaceutical composition comprising the modified immune effector cell of claim 1 and a pharmaceutically acceptable carrier and/or excipient.

59. A method for generating the modified immune effector cell of claim 1, said method comprising deleting or modifying a DNMT3A gene or gene product in the cell so that the DNMT3A-mediated de novo DNA methylation of the cell genome is inhibited.

60-88. (canceled)

89. A method of treating a disease in a subject in need thereof comprising administering to the subject an effective amount of the modified immune effector cells of claim 1 or a pharmaceutical composition comprising said modified immune effector cells and a pharmaceutically acceptable carrier and/or excipient.

90-101. (canceled)

102. A method of enhancing an antitumor activity of a T cell, comprising:
a) modifying a DNMT3A gene or gene product in the cell so that the DNMT3A-mediated de novo DNA methylation of the cell genome is inhibited; and
b) activating the STAT5 signaling pathway in the cell by either stimulating the cell with a signaling molecule or genetically modifying the cell to express a signaling molecule.

103-105. (canceled)
